SEQUENCE LISTING (4 sequences)

SEQ ID NO: 1 - protein, 37 aa, Homo sapiens
  MISC_FEATURE (37)..(37): NH2 modification
ACDTATCVTHRLAGLLSRSGGVVKNNFVPTNVGSKAF

SEQ ID NO: 2 - protein, 37 aa, Homo sapiens
  MISC_FEATURE (37)..(37): NH2 modification
ACNTATCVTHRLAGLLSRSGGMVKSNFVPTNVGSKAF

SEQ ID NO: 3 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 4 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2021/0386829 A1
(43) Pub. Date: Dec. 16, 2021

(54) COMPOSITIONS AND METHODS FOR MODULATING CGRP SIGNALING TO REGULATE INNATE LYMPHOID CELL INFLAMMATORY RESPONSES

(71) Applicants: The Broad Institute, Inc., Cambridge, MA (US); Massachusetts Institute of Technology, Cambridge, MA (US); The Brigham and Women's Hospital, Inc., Boston, MA (US)

(72) Inventors: Antonia Wallrapp, Boston, MA (US); Patrick R. Burkett, Boston, MA (US); Vijay K. Kuchroo, Boston, MA (US); Aviv Regev, Cambridge, MA (US); Samantha J. Riesenfeld, Cambridge, MA (US); Heping Xu, Cambridge, MA (US)

(21) Appl. No.: 17/052,873

(22) PCT Filed: May 6, 2019
(86) PCT No.: PCT/US2019/030911

§ 371 Date: Nov. 4, 2020

Related U.S. Application Data
(60) Provisional application No. 62/818,168, filed on Mar. 14, 2019.
(60) Provisional application No. 62/667,381, filed on May 4, 2018.

(51) Int. Cl. A61K 38/22; G01N 33/564; A61K 45/06; A61P 37/00

(52) U.S. Cl. CPC A61K 38/225; A61P 37/00; A61K 45/06; G01N 33/564

(57) ABSTRACT

The present invention provides novel compositions and methods based on the discovery of the mechanisms and gene expression programs associated with homeostatic ILC2s and proinflammatory ILC2s that drive tissue inflammation. Molecular cues were identified that modulate ILC responses to alarmins using single-cell RNA-sequencing (scRNA-seq) profiles of lung-resident ILCs at steady state and after in vivo stimulation. The neuropeptide CGRP and the CGRP receptor were identified as expressed on ILC2s. Treatment with CGRP reduces allergic lung inflammation and reduces the proliferation and expansion of ILC2 cells. The results demonstrate that CGRP signaling strongly modulates ILC2 responses and highlights the importance of neuro-immune crosstalk in allergic inflammatory responses at mucosal surfaces.

### CROSS-REFERENCE TO RELATED APPLICATIONS

This application claims the benefit of U.S. Provisional Application Nos. 62/667,381, filed May 4, 2018 and 62/818,168, filed Mar. 14, 2019. The entire contents of the above-identified applications are hereby fully incorporated herein by reference.

### STATEMENT REGARDING FEDERALLY SPONSORED RESEARCH

This invention was made with government support under grant numbers AI123516, AI056299, AI039671 and AI139536 awarded by the National Institutes of Health. The government has certain rights in the invention.

### REFERENCE TO AN ELECTRONIC SEQUENCE LISTING

The contents of the electronic sequence listing (BROD_2395WP_ST25.txt”; Size is 6,000 bytes and it was created on May 3, 2019) is herein incorporated by reference in its entirety.

### TECHNICAL FIELD

The subject matter disclosed herein is generally directed to compositions and methods targeting CGRP (Calcitonin Gene-Related Peptide) and CGRP receptor for modulating Type 2 innate lymphoid cell responses.

### BACKGROUND

Type 2 innate lymphoid cells (ILC2s) are critical for maintaining mucosal barrier functions and tissue homeostasis, and yet are also important drivers of pathologic type 2 immune responses in allergy and asthma (Cheng, D. et al. 2014, Epithelial interleukin-25 is a key mediator in Th2-high, corticosteroid-responsive asthma. American journal of respiratory and critical care medicine 190, 639-648; Huang, Y. et al. 2015, IL-25-responsive, lineage-negative KLRG1(hi) cells are multipotential ‘inflammatory’ type 2 innate lymphoid cells. Nature immunology 16, 161-169; Gudbjartsson, D. F. et al. 2009, Sequence variants affecting eosinophil numbers associate with asthma and myocardial infarction. Nature genetics 41, 342-347; Halim, T. Y. et al. 2014, Group 2 innate lymphoid cells are critical for the initiation of adaptive T helper 2 cell-mediated allergic lung inflammation. Immunity 40, 425-435; and Salimi, M. et al. 2013, A role for IL-25 and IL-33-driven type-2 innate lymphoid cells in atopic dermatitis. The Journal of experimental medicine 210, 2939-2950). Type 2 innate lymphoid cells (ILC2s) regulate the initiation of allergic tissue inflammation at mucosal surfaces, in large part due to their ability to rapidly produce effector cytokines such as IL-5 and IL-13. ILCs are also vital in maintaining tissue homeostasis by promoting epithelial cell proliferation, survival, and barrier integrity (Huang, Y. et al. 2015, IL-25-responsive, lineage-negative KLRG1(hi) cells are multipotential ‘inflammatory’ type 2 innate lymphoid cells. Nature immunology 16, 161-169). Alarmin cytokines, such as IL-25 and IL-33, activate ILC2s to promote tissue homeostasis in the face of epithelial injury, but also play critical roles in initiating allergic inflammatory responses (Moro, K. et al. 2010, Innate production of T(H)2 cytokines by adipose tissue-associated c-Kit(+)Sca-1(+) lymphoid cells. Nature 463, 540-544; Chang, Y. J. et al. 2011, Innate lymphoid cells mediate influenza-induced airway hyper-reactivity independently of adaptive immunity. Nature immunology 12, 631-638; Monticelli, L. A. et al. 2011, Innate lymphoid cells promote lung-tissue homeostasis after infection with influenza virus. Nature immunology 12, 1045-1054; and Tanay, A. & Regev, 2017, A. Scaling single-cell genomics from phenomenology to mechanism. Nature 541, 331-338). ILCs also complement adaptive immunity by providing both local and distant tissue protection during infection (Huang et al., 2018 Science 359, 114-119).

The factors that balance homeostatic and pathological ILC responses are unclear, and it remains unknown if unique subsets or functional states of ILCs mediate these homeostatic vs. pro inflammatory effects. Since there are no known markers of such functional states, it is also challenging to distinguish homeostatic from pro-inflammatory ILCs. Single-cell genomics, especially scRNA-seq (Wagner, A., Regev, A. & Yosef, N. 2016, Revealing the vectors of cellular identity with single-cell genomics. Nat Biotechnol 34, 1145-1160; and Gaublomme, J. T. et al. 2015, Single-Cell Genomics Unveils Critical Regulators of Th17 Cell Pathogenicity. Cell 163, 1400-1412), can help identify such diversity, even when changes in cell states are continuous across the cells in a population (Habib, N. et al. Div-Seq: Single-nucleus RNA-Seq reveals dynamics of rare adult newborn neurons. Science 353, 925-928, doi:10.1126/science.aad7038 (2016)), or are unique to a very small sub-population (Shekhar, K. et al. 2016, Comprehensive Classification of Retinal Bipolar Neurons by Single-Cell Transcriptomics. Cell 166, 1308-1323 e1330; and Gury-BenAri, M. et al. 2016, The Spectrum and Regulatory Landscape of Intestinal Innate Lymphoid Cells Are Shaped by the Microbiome. Cell 166, 1231-1246 e1213). Recently, scRNA-seq-based approaches identified transcriptionally distinct sub-populations within intestinal ILC subsets, demonstrating the utility of scRNA-seq in identifying previously unrecognized subpopulations and cell states within this cell type, although the functional roles of these sub-populations remain to be clarified (Monticelli, L. A. et al. 2016, Arginase 1 is an innate lymphoid-cell-intrinsic metabolic checkpoint controlling type 2 inflammation. Nature immunology 17, 656-665).

Allergic asthma is a disease of the airways that develops in response to repeated allergen exposure and is characterized by chronic inflammation leading to airway hyperreactivity and remodeling (Lambrecht and Hammad, 2015). Type 2 helper T cells (Th2 cells) have long been thought to be the main drivers of allergic lung inflammation and asthma, as they produce large amounts of the cytokines IL-4, IL-5 and IL-13, which are important for class switching to IgE, recruitment of eosinophils and goblet cell hyperplasia, respectively (Lambrecht and Hammad, 2015; Yu et al., 2014). However, recent studies have highlighted the role of type 2 innate lymphoid cells (ILC2s) to the development of such diseases. ILC2s are innate immune cells that, similar to Th2 cells, express the transcription factor Gata3 and the type 2 cytokines IL-5 and IL-13. In contrast to Th2 cells, however, ILC2s are primarily found at mucosal surfaces, including the lung, even in naïve mice, and do not express antigen-specific receptors and thus cannot respond directly to pathogens or allergens. Instead, they respond indirectly to allergens via signals from the tissue microenvironment, such as the alarmin cytokines IL-25, IL-33 or TSLP, which are released by epithelial cells upon stress or damage (Reviewed in Wallrapp et al., 2018).

As ILC2s play an important role in initiating and amplifying type 2 inflammation (Wallrapp et al., 2017), their function is tightly regulated to prevent exaggerated mucosal immune responses. Besides alarmins, an increasing array of factors have been shown to either positively or negatively regulate ILC2 function, including cytokines, cell surface receptors, and lipid mediators.

In particular, recent work has highlighted the importance of neuroimmune interactions in mucosal immunity and ILC2 function. Neurons recognize and respond to immunologically relevant molecules, including bacterial- and helminth-derived products and cytokines such as some type 2 cytokines (Cardoso et al., 2017; Chiu et al., 2013; Talbot et al., 2015). Furthermore, neurotransmitters can in turn act on both innate and adaptive immune cells to regulate their function. In particular, both peptidergic and non-peptidergic neurotransmitters are important regulators of ILC2 responses. The neuropeptides neuromedin U (NMU) and vasoactive intestinal peptide (VIP) both promote ILC2 effector function, whereas β₂-adrenergic receptor ligands (e.g., epinephrine) inhibit ILC2 proliferation and cytokine production, indicating that neurotransmitters can both stimulate and inhibit ILC2-driven responses (Cardoso et al., 2017; Klose et al., 2017; Moriyama et al., 2018; Nussbaum et al., 2013; Talbot et al., 2015; Wallrapp et al., 2017).

Given the increased prevalence and epidemic rise in allergy and asthma in the last two decades, identifying the molecular pathways that regulate ILC2s during allergic responses is an important area of inquiry.

Citation or identification of any document in this application is not an admission that such document is available as prior art to the present invention.

### SUMMARY

In one aspect, the present invention provides for a method of treating a disease associated with an innate lymphoid cell (ILC) Type 2 inflammatory response comprising administering to a subject in need thereof a therapeutically effective amount of α-CGRP or functional derivative thereof; or a α-CGRP receptor agonist. In certain embodiments, the innate lymphoid cell (ILC) Type 2 inflammatory response is an IL-33 mediated response. In certain embodiments, the disease triggers epithelial cells to release IL-33 and induce an innate lymphoid cell (ILC) Type 2 inflammatory response. In certain embodiments, the innate lymphoid cell (ILC) Type 2 inflammatory response is an IL-25+ neuromedin U (NMU) mediated response. In certain embodiments, the disease triggers epithelial cells to release IL-25 and neurons to release NMU, inducing an innate lymphoid cell (ILC) Type 2 inflammatory response. In certain embodiments, the innate lymphoid cell (ILC) Type 2 inflammatory response comprises the release of a neurotransmitter from stimulated neurons. In certain embodiments, the neurotransmitter is NMU or vasoactive intestinal peptide (VIP).

In certain embodiments, the method further comprises administering a glucocorticoid, wherein the glucocorticoid is co-administered or administered after the therapeutically effective amount of α-CGRP or derivative thereof, or the α-CGRP receptor agonist. In certain embodiments, the method further comprises administering one or more agonists of one or more genes selected from the group consisting of PD-1, TIM-3, LILRB4, CD39, GITR, wherein the one or more agonists are co-administered or administered after the therapeutically effective amount of α-CGRP or derivative thereof, or the α-CGRP receptor agonist. In certain embodiments, the agonist is an agonist antibody, small molecule or ligand, such as a GITR agonist antibody, GITR ligand (GITRL), or PD-L1.

In certain embodiments, the disease is an allergic inflammatory disease. In certain embodiments, the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome). In certain embodiments, the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma. In certain embodiments, the allergy is to an allergen selected from the group consisting of food, pollen, mold, dust mites, animals, and animal dander. In certain embodiments, IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon. In certain embodiments, the arthritis is selected from the group consisting of osteoarthritis, rheumatoid arthritis and psoriatic arthritis.

In certain embodiments, the treatment is administered to a mucosal surface. In certain embodiments, the treatment is administered to the lung, nasal passage (e.g., intranasally), trachea, gut, intestine, or esophagus. In certain embodiments, the treatment is administered by aerosol inhalation. In certain embodiments, the treatment is administered by a time release composition.

In another aspect, the present invention provides for a method of treating a disease by enhancing an innate lymphoid cell (ILC) Type 2 inflammatory response comprising administering to a subject in need thereof a therapeutically effective amount of an agent capable of antagonizing α-CGRP receptor signaling or blocking the α-CGRP receptor interaction with α-CGRP. In certain embodiments, the agent comprises a therapeutic antibody, antibody fragment, antibody-like protein scaffold, aptamer, nucleic acid molecule, genetic modifying agent, protein or small molecule. In certain embodiments, the agent binds to the α-CGRP receptor or α-CGRP. In certain embodiments, the method further comprises administering one or more inhibitors of one or more genes selected from the group consisting of PD-1, TIM-3, LILRB4, CD39, GITR and PD-L1. In certain embodiments, the one or more inhibitors comprises an antibody or small molecule specific for PD-1, TIM-3, LILRB4, CD39, GITR, or PD-L1. In certain embodiments, the one or more inhibitors comprises Nivolumab, Pembrolizumab, Atezolizumab, 6-N,N-Diethyl-d-β-γ-dibromomethylene adenosine triphosphate (ARL 67156), 8-thiobutyladenosine 5′-triphosphate (8-Bu-S-ATP), polyoxymetate-1 (POM-1), or α,β-methylene ADP (APCP). In certain embodiments, the disease is cancer or an infection.

In another aspect, the present invention provides for a method of treatment for a subject in need thereof suffering from allergic inflammation comprising: detecting in ILC2s obtained from the subject the expression or activity of an innate lymphoid cell type 2 inflammatory gene signature comprising one or more genes or polypeptides selected from the group consisting of: (IL-33+CGRP signature) Sos1, Egfr, Tph1, P2ry1, Far1, Plin2, Alox5, Pparg, Ikzf1, Ier3, Rilpl2, Stap1, Gimap5, Odc1, Smox, Calca, Ramp3, Rora, I17r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd41, Jag1, Zfp36l1, Lmo4, Il13, I16, I14ra, Prdm1, Arg1, Zeb2, Srgap3, Ptger4, Pcsk1, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1 and Serpinb9; or (IL-25+NMU signature) Fosb, Btg2, Lpcat2, Sdc4, Csf2, Dgat2, Calca, Areg, Pim2, Zfp36l1, Nr4a1, Cd81, Ly6a, Lgmn, Il13, Il5, Klrg1, Batf, Pycard, Pdcd1, Lgals3, Anaxa2, Ctla4, Il1r2, Tox2, Tnfrsf8, Mt1, Tff1, Lilrb4a and H2-Ab1; or (IL-25+NMU signature) Calca, Areg, Anxa1, Anxa2, Ccl1, Ccl5, Ccr2, Ccr7, Ccr8, Cd200r1, Cd3d, Cd47, Cd48, Cd81, Csf2, Ctla4, Fas, H2-Aa, H2-Ab1, H2-Q8, H2-T23, Il13, Il1r2, Il2rb, Il5, Il6, Klrg1, Lat, Lgals3, Lilrb4a, Ltb, Mif, Ms4a4b, Nmur1, Pdcd1, Pgk1, Ptger2, Ramp1, Sdc4, Sema4a, Sepp1, Stab2, Tff1, Tmem176a, Tnfrsf4, Tnfrsf8, Tnfsf8, Vsir, Nmu, 2810417H13Rik, AA467197, Alox5, Arg1, Atf4, Batf, Bcl2a1b, Blk, Btg1, Cox5b, Cox6c, Crip1, Dgat1, Dgat2, Dusp1, Ets1, Fos, Fosb, Furin, Gadd45b, Gsto1, Hint1, Ier2, Irf4, Klf3, Klf4, Lgmn, Lpcat2, Mcm3, Mt1, Myl6, Ndufa4, Nfkbia, Nfkbid, Nfkbiz, Nop56, Nr4a1, Prdx4, S100a4, S100a6, Serpinb6a, Snrpd3, Sptssa, Tph1, Vim, Zfp36 and Zfp36l1; or (IL-25+NMU signature) Anxa2, Lgals3, Ctla4, Batf, Cd47, Tnfrsf8, AA467197, S100a6, Prdx4, Gsto1, Il1r2, Lgmn, Mt1, Tff1, Ccr7, Irf4, 116, Tnfrsf4, H2-T23, Lilrb4a, Fas, Ets1, Ramp1, Nmur1, Dgat2, Calca, Ccl5, Btg1, Nr4a1, Klf3, Klf4, Csf2, Stab2, Sdc4, Ccr2, Fosb, Zfp36l1, Lpcat2 and Ltb; and treating the subject with α-CGRP or functional derivative thereof, or an agonist of the α-CGRP receptor if the inflammatory signature is detected. In certain embodiments, the inflammatory signature genes are up and down regulated according to FIG. 16 (IL-33+CGRP signature) or according to FIGS. 4K and/or 16H of WO2018175924A1 (IL-25+NMU signature). The IL-33+CGRP signature is a signature that includes genes differentially expressed between ILC2s treated with IL-33 alone and IL-33+CGRP. Thus, in certain embodiments, the IL-33+CGRP signature is an inflammatory signature when the genes upregulated after treatment with IL-33 as compared to IL-33+CGRP are upregulated (Sos1, Egfr, Tph1, P2ry1, Far1, Plin2, Alox5, Pparg, Ikzf1, Ier3, Rilpl2, Stap1, Gimap5, Il13, Il6, Il4ra, Prdm1, Arg1, Zeb2, Srgap3, Ptger4, Pcsk1) and the genes downregulated after treatment with IL-33 as compared to IL-33+CGRP are downregulated (Odc1, Smox, Calca, Ramp3, Rora, Il7r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd4l, Jag1, Zfp36l1, Lmo4, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1, Serpinb9) in ILC2s obtained from the subject.

The IL-25+NMU signature is a signature that includes genes differentially expressed between ILC2s treated with IL-25 alone or no treatment and IL-25+NMU. Thus, in certain embodiments, the IL-25+NMU signature is an inflammatory signature when the genes positively correlated to the inflammatory signature are upregulated (Anxa2, Lgals3, Ctla4, Batf, Cd47, Tnfrsf8, AA467197, S100a6, Prdx4, Gsto1, Il1r2, Lgmn, Mt1, Tff1, Ccr7, Irf4, Il6, Tnfrsf4, H2-T23, Lilrb4a, Fas, Ets1, Ramp1, Il5 and Areg; or Cd81, Ly6a, Lgmn, Il13, Il5, Klrg1, Batf, Pycard, Pdcd1, Lgals3, Anaxa2, Ctla4, Il1r2, Tox2, Tnfrsf8, Mt1, Tff1, Lilrb4a and H2-Ab1) and the genes negatively correlated to the inflammatory signature are downregulated (Nmur1, Dgat2, Calca, Ccl5, Btg1, Nr4a1, Klf3, Klf4, Csf2, Stab2, Sdc4, Ccr2, Fosb, Zfp36l1, Lpcat2, Btg2 and Ltb; or Fosb, Btg2, Lpcat2, Sdc4, Csf2, Dgat2, Calca, Areg, Pim2, Zfp36l1, Nr4a1) in ILC2s obtained from the subject.

In another aspect, the present invention provides for a method of detecting and/or monitoring an immune response comprising detecting in ILC2s the expression of one or more genes selected from the group consisting of. Calca, Ramp1, Calcrl, and Ramp3; or Sos1, Egfr, Tph1, P2ry1, Far1, Plin2, Alox5, Pparg, Ikzf1, Ier3, Rilpl2, Stap1, Gimap5, Odc1, Smox, Calca, Ramp3, Rora, Il7r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd4l, Jag1, Zfp36l1, Lmo4, 1113, 116, Il4ra, Prdm1, Arg1, Zeb2, Srgap3, Ptger4, Pcsk1, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1 and Serpinb9; or Arg1, Ly6a, Stab1, Ptger4, Maf, Tph1, Traip, Kdm8, Birc5, Mki67, Crem, Fosl2, Odc1, Smox, Nr3c1, Rora, Lmo4, Ikzf3, Il7r, Il1rl1, Crlf2, Il17rb, Xbp1, Itk, Ccr4, Icos, Irf4, Pdcd1, Ctla2a, Fgl2, Gp49a, Nt5e, Tnfrsf9, Tnfrsf18, Lilrb4, Tnfaip3, Pde4d, Nmb, Calca, Ramp3, Serpinb9, Hif1a, Egln3. In certain embodiments, the immune response is monitored in a subject administered an allergic challenge. In certain embodiments, the immune response is monitored in a subject undergoing treatment for an allergic inflammatory disease.

In certain embodiments, the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome). In certain embodiments, the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma. In certain embodiments, the allergy is to an allergen selected from the group consisting of foods, pollen, mold, dust mites, animals, and animal dander. In certain embodiments, IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon. In certain embodiments, the arthritis is selected from the group consisting of osteoarthritis, rheumatoid arthritis and psoriatic arthritis. In certain embodiments, the immune response is monitored in a subject suffering from cancer.

In another aspect, the present invention provides for a medical device comprising a therapeutically effective amount of α-CGRP or functional derivative thereof. In certain embodiments, the device further comprises a glucocorticoid. In certain embodiments, the device is a nasal spray.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Differential expression of Calca and Ramp1 following alarmin activation. (A, B) ILCs from the PBS, IL-25 and IL-33 condition are represented on tSNE plots colored by treatment (A) and cluster (B). (C) Violin plot showing the expression of Calca in lung ILCs isolated from PBS-, IL-25- and IL-33-treated mice. (Calca, PBS vs. IL-25, p<3.43E-95; PBS vs. IL-33, p<9.23E-60) (D, E) Expression of Calca (D) and Ramp1 (E) by cluster. (Calca, cluster 9 vs. cluster 3, p<3.54E-153; Ramp1, cluster 8 vs. cluster 3, p<4.37E-71) Ramp1 is significantly lower in cluster 8, which is composed of KLRG1^{hi} ST2⁻ pro-inflammatory ILCs. Significance values were determined using the zero-inflated negative binomial model.

FIG. 2—Lung ILCs express CGRP (Calca) during airway inflammation. (A, B) Ramp1 (left), Calcrl (middle) and Calca (right) expression in different cell types isolated from the lungs of naive mice (A) or following treatment with either HDM (immune cells) or IL-33 (neurons) (B), as assessed by qPCR.

FIG. 3—CGRP reduces IL-5 and IL-13 production in ILCs in vitro. Lung-derived ILCs were cultured with IL-33 or IL-33+CGRP and analyzed after three days. (A) 115 and Il13 expression was determined by qPCR. (B) IL-5 and IL-13 concentration in the supernatant was analyzed by LegendPlex. (C) Areg expression in ILCs, as assessed by qPCR. Mean is indicated. Error bars, s.e.m.*p<0.05 by two-tailed t-test.

FIG. 4—ILC-derived CGRP reduces IL-5 and IL-13 production in ILCs in vitro. Lung-derived ILCs from Calca Het or Calca KO mice were cultured with PBS or IL-33 for three days. Il13 expression was analyzed by qPCR. Mean is indicated. Error bars, s.e.m.

FIG. 5—CGRP attenuates IL-33 induced airway inflammation. Mice received IL-33 or IL-33+CGRP intranasally for three consecutive days and were analyzed one day after the last treatment. (A) CGRP reduces 115 and 1113 expression in lung tissue, as assessed by qPCR. (B) IL-5 and IL-13 concentration is lower in the BALF of IL-33+CGRP treated mice. IL-5 and IL-13 concentrations were determined by LegendPlex. (C, D) Eosinophil frequency and number are reduced in the presence of CGRP. Eosinophil frequencies and numbers from lungs (C) and bronchoalveolar lavage fluid (BALF) (D) were assessed by flow cytometry. Data points are individual mice (n=6) from two independent experiments. Mean is indicated. Error bars, s.e.m.*p<0.05 by two-tailed t-test.

FIG. 6—CGRP inhibits IL-33-induced proliferation in a dose-dependent manner. ILCs were labeled with CellTrace Violet for flow cytometric analysis.

FIG. 7—DSS-induced colitis in CGRP WT, Het and KO mice.

FIG. 8—Regulators of ILC function. (A) Differential gene expression analysis across clusters and conditions identifies potential novel regulators of ILC function. Expression of several differentially expressed genes is shown by condition. The size of the dot reflects the percentage of positive cells within each condition and the color of the dot shows the expression level within the positive cells. (B) Gene expression patterns identified by scRNA-seq were validated by flow cytometry. Expression of Nr4a1, CTLA4, IL1R2, and CD30 (Tnfrsf8) on ILCs from mice treated with PBS (grey, closed histogram) versus one of the treatments (blue, closed histogram), as well as an FMO control (dashed open histogram) is shown. The mean fluorescence intensity is indicated for Nr4a1 and the frequency of positive ILCs in PBS (grey) and in the indicated condition (blue) is shown for the other proteins.

FIG. 9—ILC2s express the CGRP receptor Ramp1/Calcrl and its ligand CGRP. (A) Violin plots show expression of the indicated neuropeptide receptors (x axis) in lung ILCs isolated from PBS-treated mice as determined by scRNA-seq. (B) Schematic illustrating CGRP and adrenomedullin receptor components. Both receptors share the seven transmembrane domain protein Calcrl. Ramp1 and Calcrl make up the CGRP receptor, while Ramp3 and Calcrl make up the adrenomedullin receptor. (C) Ramp1, Ramp3, and Calcrl gene expression. tSNE plots show individual of lung ILCs (dots) isolated from PBS-, IL-25- or IL-33-challenged mice in a two dimensional reduced representation of the top 22 PCs. Color indicates relative expression of the indicated gene. (D) Expression of Ramp1, Ramp3, Calcrl in the indicated cell populations isolated from the lungs of naïve or IL-33-challenged mice, determined by qPCR analysis. Data points are technical replicates (n=3). Data are representative of two independent experiments. (E) CGRP-GFP expression in the indicated immune cell populations isolated from the lungs of naïve CGRP-GFP reporter mice. Data points are biological replicates (n=3 for ILC2s and n=2 for other immune cell populations). (F) tSNE shows expression of the gene encoding CGRP (Calca) in ILCs isolated from PBS-, IL-25- or IL-33-challenged mice. Color indicates relative gene expression. (G) Frequency of CGRP-GFP+ lung ILC2s isolated from CGRP-GFP reporter mice after overnight culture with IL-7 and subsequent stimulation with PBS or IL-33 for 9 hr, as determined by flow cytometry. Data points are biological replicates from two independent experiments.

FIG. 10—(A-B) Previously generated (Wallrapp et al., 2017) scRNA-seq transcriptional profiles of lung ILCs (dots) isolated from PBS-, IL-25, or IL-33 challenged mice. tSNE plots are colored by treatment condition (A) or cluster (B) and shown here for reference. (C) Expression of Ramp2 in the indicated cell populations isolated from the lungs of naïve or IL-33-challenged mice, as determined by qPCR analysis. Data points are technical replicates (n=3). Data are representative of two independent experiments. (D) Violin plots show expression of the indicated neuropeptides (x axis) in lung ILCs isolated from PBS-treated mice as determined by scRNA-seq. (E) Histogram shows expression of CGRP-GFP in ILC2s cultured over night with IL-7 and stimulated with PBS (grey) or IL-33 (blue) for 9 hr and the percent of CGRP-GFP+ cells in either condition is indicated.

FIG. 11—CGRP-GFP expression in the indicated cell populations isolated from the lungs of CGRP-GFP reporter mice (blue) or wild type littermate controls (grey) was determined by flow cytometry and the percent of CGRP-GFP+ cells is indicated.

FIG. 12—CGRP inhibits type 2 cytokine expression and proliferation of ILC2s in vitro. (A) Schematic illustrating the experimental method. ILCs are isolated from the lungs of C57BL/6J mice by fluorescence activated cell sorting (FACS) and cultured with IL-7 or IL-7+IL-33 with medium or CGRP. (B) Expression of Il5 and Il13 in ILC2s cultured over night with IL-7 and CGRP (top) or IL-33+CGRP (bottom) for 6 hours was determined by qPCR. Data points are technical replicates (n=2). Data are representative of two independent experiments. (C) Areg mRNA expression in ILC2s cultured as described in (B) was determined by qPCR. Data points are technical replicates (n=2). Data are representative of two independent experiments. (D) Expression of Il13 and Il5 in ILCs was determined by qPCR. ILCs were cultured with IL-33 and IL-33+CGRP for 3 days. Data points are the average of technical replicates from four independent experiments. (E) IL-13 and IL-5 concentration in supernatant of ILCs cultured for 3 days with IL-33 or IL-33+CGRP, as determined by LegendPlex. Data points are averages from technical replicates of four independent experiments. (F) Flow cytometric analysis of IL-13 and IL-5 expression in ILCs cultured for 3 days with IL-33 or IL-33+CGRP. Gating strategy (top), frequency (bottom) and geometric mean fluorescence intensity (MFI; bottom) of IL-5 and IL-13 are shown. Data points are technical replicates (n=2). Data are representative of two independent experiments. (G) Expression of Il13 and Il5 mRNA in ILCs cultured with IL-25+NMU or IL-25+NMU+CGRP for 3 days. Data points are technical replicates from one experiment. Data are representative of three independent experiments. (H) Concentration of IL-13 and IL-5 in supernatant from ILCs cultured with IL-25+NMU or IL-25+NMU+CGRP for 3 days, as determined by LegendPlex. Data points are technical replicates from one experiment. Data are representative of three independent experiments. (I) ILCs were labeled with CellTrace Violet and cultured with IL-7 or IL-33 and 100 pM or 100 nM CGRP for 3 days. Histograms show CellTrace Violet dye expression in ILCs and gating strategy used to determine proliferating ILCs (left). Graph shows frequency of proliferating ILCs in the indicated conditions (right) from 2-3 independent experiments. Data shown are the mean±s.e.m. *P<0.05; **P<0.01; ***P<0.001; ns, not significant.

FIG. 13—Intracellular cytokine staining for IL-5 and IL-13 in ILCs cultured over night with IL-7 and 8 hours with IL-33 or IL-33+CGRP. Frequency (left) and gating strategy (right) are shown. Data points are technical replicates (n=2). Data are representative of two independent experiments. Data shown are the mean±s.e.m. *P<0.05; **P<0.01; ***P<0.001; ns, not significant.

FIG. 14—Inflammatory ILC2s express less Ramp1 and do not respond to CGRP. (A) Violin plots show expression of Klrg1, Il1rl1, Il5 and Il13 by cluster (x axis). (B) Violin plot shows expression of Ramp1 and Ramp3 by cluster (x axis). (C) Schematic showing experimental method. C57BL/6J mice receive IL-25 intraperitoneally for three consecutive days. One day after the last treatment, natural ILC2s (ST2+KLRG1−ILCs; nILC2s) and inflammatory ILC2s (ST2−KLRG1+ILCs; iILC2s) are isolated. (D) Expression of Ramp1, Ramp3 and Calcrl in different ILC subsets is shown. Data points are technical replicates from one experiment. Data are representative of two independent experiments. (E) Schematic showing experimental method. C57BL/6J mice receive IL-25 intraperitoneally for three consecutive days. One day after the last treatment, inflammatory ILC2s (ST2−KLRG1+ILCs; iILC2s) are isolated and cultured in vitro with IL-33, IL-33+CGRP, IL-25 or IL-25+CGRP for 6 hours. (F-G) Il5 and Il13 expression in iILC2s cultured with IL-33 or IL-33+CGRP (F) or IL-25 or IL-25+CGRP (G). Data points are technical replicates from one experiment. Data are representative of two independent experiments. Data shown are the mean s.e.m. *P<0.05; **P<0.01; ***P<0.001; ns, not significant.

FIG. 15—Gating strategy for the isolation of natural ILC2s (ST2+KLRG1−ILCs; nILC2s) and inflammatory ILC2s (ST2−KLRG1+ILCs; iILC2s) from the lung is shown.

FIG. 16—CGRP modulates ILC activation and regulatory gene module. (A) Overview of selected differentially expressed genes with a fold change in expression of at least 1.5 between ILCs cultured with IL-33 and IL-33+CGRP. (B) GO term enrichment analysis for differentially expressed genes in ILCs cultured with IL-33 or IL-33+CGRP. (C) Differentially expressed pro-inflammatory and regulatory genes with a fold change in expression of at least 1.5 between ILCs cultured with IL-33 or IL-33+CGRP. (D) tSNE plot shows ILCs (dots) colored by score of CGRP signature. (E) Violin plots show CGRP gene signature score by cluster (x axis).

FIG. 17—(A) Expression of Calca, Ramp3, Odc1 and Arg1 is shown in ILCs cultured with IL-7 and IL-7+CGRP (top) or IL-33 and IL-33+CGRP (bottom). (B) Selected differentially expressed genes with a fold change in expression of at least 1.5 between ILCs cultured with IL-7 or IL-7+CGRP. (C) GO term enrichment analysis for differentially expressed pathways in ILCs cultured with IL-7 or IL-7+CGRP. (D) Violin plots show expression of Klrg1 in ILC2s by cluster (x axis). (E) tSNE plot shows ILCs (dots) colored by score of a version of the CGRP signature without Calca and Ramp3.

FIG. 18—CGRP dampens IL-33-induced airway inflammation. (A) Schematic illustrating experimental model. PBS, CGRP, IL-33 or IL-33+CGRP were administered intranasally to C57BL/6J mice on three consecutive days and mice were analyzed one day after the last treatment. (B) Flow cytometric analysis of lung ILCs from mice challenged with PBS, CGRP, IL-33 or IL-33+CGRP. Frequency (left) and number (right) of lung ILCs are shown. (C) Frequency of ki67+ lung ILCs. (D) Frequency of IL-5+ (top) and IL-13+ (bottom) lung ILCs by flow cytometry. (E) Expression of Il5 (top) and Il13 (bottom) mRNA in lung tissue isolated from mice from the different treatment conditions, as determined by qPCR. (F) Concentration of IL-5 (top) and IL-13 (bottom) in the BALF of mice from the different treatment conditions. (G) Eosinophil frequency (top) and number (bottom) in the BALF of mice from the different treatment conditions. (H) Representative H&E staining of lung sections from mice challenged with PBS, CGRP, IL-33 or IL-33+CGRP (left). Lung sections were scored for disease severity in a blinded manner. Graph (right) shows severity score for individual mice (n=9) from three independent experiments. 0, normal; 1, very mild; 2, mild; 3, moderate; 4, severe. (I) Airway resistance was assessed in mice challenged with IL-33 or IL-33+CGRP in response to challenge with increasing doses of methacholine. Data points represent the mean of individual mice from two independent experiments (IL-33, n=9; IL-33+CGRP, n=9). Data points are individual mice pooled from three independent experiments (n=9) in panels B and E-G and pooled from two independent experiments (n=6) in panels C and D. Data shown are the mean±s.e.m. *P<0.05; **P<0.01; ***P<0.001; ns, not significant.

FIG. 19—(A) Concentration of IL-5 and IL-13 in lung tissue from mice challenged with PBS, CGRP, IL-33 or IL-33+CGRP, as determined by LegendPlex. Data points are individual mice (n=6) pooled from two independent experiments. (B) Eosinophil frequency (left) and number (right) in lung tissue of mice from the different treatment conditions. Data points are individual mice (n=9) pooled from three independent experiments. Data shown are the mean s.e.m. *P<0.05; **P<0.01; ***P<0.001; ns, not significant.

FIG. 20—CGRP ameliorates IL-25+NMU-induced airway inflammation. (A) Schematic illustrating experimental approach. IL-25, IL-25+CGRP, IL-25+NMU or IL-25+NMU+CGRP were administered intranasally to C57BL/6J mice for three consecutive days. Mice were analyzed one day after the last treatment. (B) Frequency (left) and number (right) of lung ILCs, as determined by flow cytometry. (C) Flow cytometric analysis of ILC proliferation by intracellular staining for the proliferation marker Ki67. Frequency of Ki67+ ILCs is shown. (D) Il5 and Il13 mRNA expression in lung tissue. (E) Concentration of IL-5 and IL-13 in BALF, determined by LegendPlex. (F) Frequency (left) and number (right) of eosinophils in BALF, determined by flow cytometry. Data points are individual mice (n=6) pooled from two independent experiments for all panels, except panel C which shows individual mice (n=3) from one experiment. Data shown are the mean±s.e.m. *P<0.05; **P<0.01; ***P<0.001; ns, not significant.

FIG. 21—Concentration of IL-5 and IL-13 in lung tissue, determined by LegendPlex. Data points are individual mice (n=6) pooled from two independent experiments in panels A and B. Data shown are the mean±s.e.m. *P<0.05; **P<0.01; ***P<0.001; ns, not significant.

FIG. 22—CGRP negatively regulates ILC2 responses in vivo independent of T cells. (A) Nasal administration of IL-33 or IL-33+CGRP to RAG2 KO mice for three consecutive days. Mice were analyzed one day after the last treatment. (B) Frequency (left) and number (right) of lung ILCs isolated from mice challenged with IL-33 or IL-33+CGRP. (C) Frequency of IL-5+(left) and IL-13+(right) lung ILCs, as determined by flow cytometry. (D) Expression of Il5 and Il13 mRNA in lung tissue. (E-F) Concentration of IL-5 and IL-13 in lung tissue (E) and BALF (F). (G-H) Frequency (top) and number (bottom) of eosinophils in lung tissue (G) and BALF (H) from mice challenged with IL-33 or IL-33+CGRP. Data points are individual mice (n=10) pooled from three independent experiments. Data shown are the mean±s.e.m. *P<0.05; **P<0.01; ***P<0.001; ns, not significant.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International application number PCT/US2018/024082, published as WO2018175924A1 on Sep. 27, 2018.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods and compositions for modulating an innate immune response, in particular an innate lymphoid cell class 2 innate immune response by modulating activity of CGRP signaling. Embodiments disclosed herein also provide for methods of monitoring an innate lymphoid cell class 2 innate immune response in response to disease or treatment.

Type 2 innate lymphoid cells (ILC2s) both contribute to mucosal homeostasis and initiate pathologic inflammation (type 2 immune responses). Type 2 inflammation (e.g., allergic asthma) involves the interaction of multiple immune cell types. The signals that direct ILC2s to promote homeostasis versus inflammation were previously unknown. While both IL-33 and IL-25 promote ILC activation in vivo, IL-33 induces robust ILC proliferation, whereas ILCs activated with IL-25 do not proliferate as robustly.

Previous studies related to type 2 immune responses regulated by IL-25 in conjunction with the neuropeptide receptor NMUR1 and the neuropeptide NMU (Wallrapp, et al., The neuropeptide NMU amplifies ILC2-driven allergic lung inflammation, Nature. 2017 Sep. 21; 549(7672):351-356. doi: 10.1038/nature24029. Epub 2017 Sep. 13). Lung-resident ILCs were profiled using single-cell RNA-seq at steady state and after in vivo stimulation with the alarmin cytokines IL-25 and IL-33. ILC2s were transcriptionally heterogeneous after activation, with subpopulations distinguished by expression of proliferative, homeostatic, and effector genes. The neuropeptide receptor Nmur1 was preferentially expressed by ILC2s at steady state and after IL-25 stimulation. Neuromedin U (NMU), the ligand of Nmur1, activated ILC2s in vitro, and in vivo co-administration of NMU with IL-25 dramatically amplified allergic inflammation. ILC2s express several neuropeptide receptors and NMUR1 was studied in detail. Both functional analysis and scRNA-seq-based approaches demonstrated that Nmur1 modulates alarmin- and allergen-driven ILC2 responses. Thus, blocking this neuroimmune pathway may inhibit development of pro-allergic immune cells and mitigate many of the symptoms observed in patients during allergic responses.

It is an objective of the present invention to identify molecular cues that modulate ILC responses to alarmins (e.g., for therapeutic applications). As neuroimmune interactions have emerged as critical modulators of allergic inflammation, and type 2 innate lymphoid cells (ILC2s) are an important cell type for mediating these interactions Applicants attempted to identify additional neuroimmune pathways that may modulate ILC2 responses and can be targeted therapeutically. To identify novel neuropeptides that regulate ILC function, Applicants analyzed the lung-resident ILC2 single cell RNA-seq (scRNA-seq) profiles for the expression of both neuropeptides and neuropeptide receptors, particularly those that are differentially expressed between homeostatic and inflammatory lung-derived ILCs (see, WO2018175924A1 and Wallrapp, et al., 2017). One of the neuropeptides that was expressed in ILCs from all treatment conditions and was upregulated after alarmin treatment was CGRP (encoded by the Calca gene). CGRP has previously been identified in other cell types (see, e.g., Bonner et al., J Allergy Clin Immunol December 2010).

Here Applicants demonstrate for the first time a role for the neuropeptide CGRP in type 2 immune responses (IL-33 and IL-25+NMU mediated). Applicants show that ILC2s not only express the receptor for the neuropeptide CGRP but also CGRP itself. Applicants further demonstrate that CGRP limits both type 2 cytokine production (IL-13 and IL-5) and ILC2 proliferation in vitro. CGRP also induces a unique regulatory gene expression profile in lung-resident ILCs. In an in vivo model of lung inflammation, treatment with CGRP restrains ILC2-dependent airway inflammation, indicating that CGRP is a central negative regulator of ILC2-mediated allergic inflammation.

Specifically, CGRP potently inhibited alarmin-driven type 2 cytokine production and proliferation by ILC2s both in vitro and in vivo, and this inhibition was independent of adaptive immune cells. Treatment of ILC2s with CGRP reduces allergic lung inflammation and reduces the proliferation and expansion of specific ILC2 subsets. Administration of CGRP attenuates IL-33 induced airway inflammation, as well as inflammation induced by IL-25+NMU. CGRP induced marked changes in ILC2 gene expression, promoting expression of a co-inhibitory gene module that has also been observed in dysfunctional T cells. By analyzing differentially expressed genes after CGRP stimulation in vitro Applicants developed a CGRP-specific gene signature and found that a population of ILCs scored highly for this signature after stimulation by alarmins in vivo, indicating that endogenous CGRP is a critical negative regulator of ILC2 responses in vivo. CGRP induced genes provide additional targets that are upregulated in response to CGRP treatment and that may be modulated to regulate ILC2 immune responses.

The discovery presented herein highlights the importance of neuro-immune crosstalk in allergic inflammatory responses at mucosal surfaces. Moreover, Applicants have discovered novel regulatory mechanisms for modulating the balance between tissue protective ILCs and tissue inflammatory cells. In certain embodiments, the methods and compositions described herein may be used to shift the balance of ILC2 responses in order to treat inflammatory allergic diseases and cancer.

It is an objective of the present invention to modulate ILC2 immune responses and cell states using CGRP either alone or in combination with other treatments. It is another objective of the present invention to enhance current treatments for diseases associated with aberrant ILC2 inflammatory responses. It is another objective, to modulate ILC2 immune responses using CGRP in combination with agents currently in use for the modulation of immune responses.

### Gene Signatures

As used herein a “signature” may encompass any gene or genes, protein or proteins (e.g., gene products), or epigenetic element(s) whose expression profile or whose occurrence is associated with a specific cell type, subtype, or cell state of a specific cell type or subtype within a population of cells (e.g., inflammatory or homeostatic ILC2 cells). In certain embodiments, the expression of an ILC2 signature (e.g., inflammatory or CGRP signature) is dependent on epigenetic modification of the genes or regulatory elements associated with the signatures. Thus, in certain embodiments, use of signature genes includes epigenetic modifications that may be detected or modulated. For ease of discussion, when discussing gene expression, any of gene or genes, protein or proteins, or epigenetic element(s) may be substituted. As used herein, the terms “signature”, “expression profile”, or “expression program” may be used interchangeably (e.g., expression of genes, expression of gene products or polypeptides). It is to be understood that also when referring to proteins (e.g. differentially expressed proteins), such may fall within the definition of “gene” signature. Levels of expression or activity may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and/or down-regulated genes that are representative of a cell type or subtype. A gene signature as used herein, may also refer to any set of up- and/or down-regulated genes between different cells or cell (sub)populations derived from a gene-expression profile. For example, a gene signature may comprise a list of genes differentially expressed in a distinction of interest.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g. ILC2 samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. Not being bound by a theory the signatures of the present invention may be microenvironment specific, such as their expression in a particular spatio-temporal context. Not being bound by a theory, signatures as discussed herein are specific to a particular pathological context. Not being bound by a theory, a combination of cell subtypes having a particular signature may indicate an outcome. Not being bound by a theory, the signatures can be used to deconvolute the network of cells present in a particular pathological condition. Not being bound by a theory the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular pathological condition (e.g. inflammation), or linked to a particular outcome or progression of the disease, or linked to a particular response to treatment of the disease.

The signature according to certain embodiments of the present invention may comprise or consist of one or more genes, proteins and/or epigenetic elements, such as for instance 1, 2, 3, 4, 5, 6, 7, 8, 9, 10 or more. In certain embodiments, the signature may comprise or consist of two or more genes, proteins and/or epigenetic elements, such as for instance 2, 3, 4, 5, 6, 7, 8, 9, 10 or more. In certain embodiments, the signature may comprise or consist of three or more genes, proteins and/or epigenetic elements, such as for instance 3, 4, 5, 6, 7, 8, 9, 10 or more. In certain embodiments, the signature may comprise or consist of four or more genes, proteins and/or epigenetic elements, such as for instance 4, 5, 6, 7, 8, 9, 10 or more. In certain embodiments, the signature may comprise or consist of five or more genes, proteins and/or epigenetic elements, such as for instance 5, 6, 7, 8, 9, 10 or more. In certain embodiments, the signature may comprise or consist of six or more genes, proteins and/or epigenetic elements, such as for instance 6, 7, 8, 9, 10 or more. In certain embodiments, the signature may comprise or consist of seven or more genes, proteins and/or epigenetic elements, such as for instance 7, 8, 9, 10 or more. In certain embodiments, the signature may comprise or consist of eight or more genes, proteins and/or epigenetic elements, such as for instance 8, 9, 10 or more. In certain embodiments, the signature may comprise or consist of nine or more genes, proteins and/or epigenetic elements, such as for instance 9, 10 or more. In certain embodiments, the signature may comprise or consist of ten or more genes, proteins and/or epigenetic elements, such as for instance 10, 11, 12, 13, 14, 15, or more. It is to be understood that a signature according to the invention may for instance also include genes or proteins as well as epigenetic elements combined.

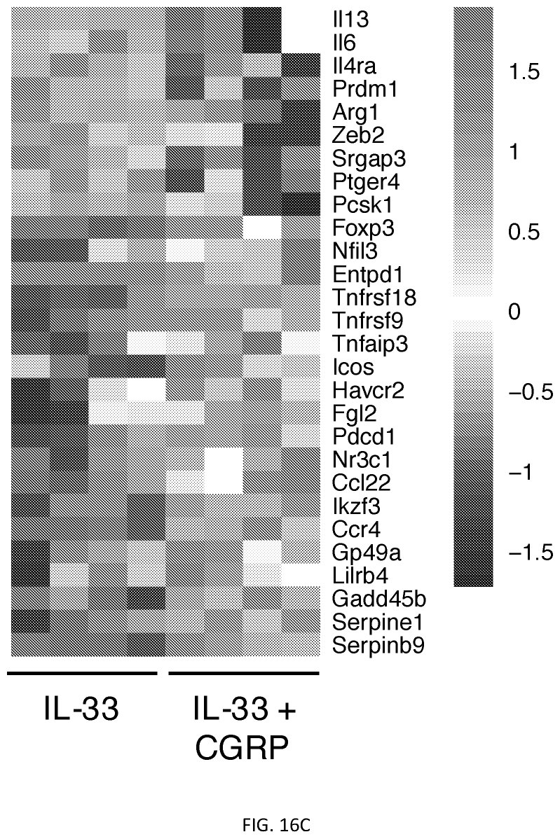

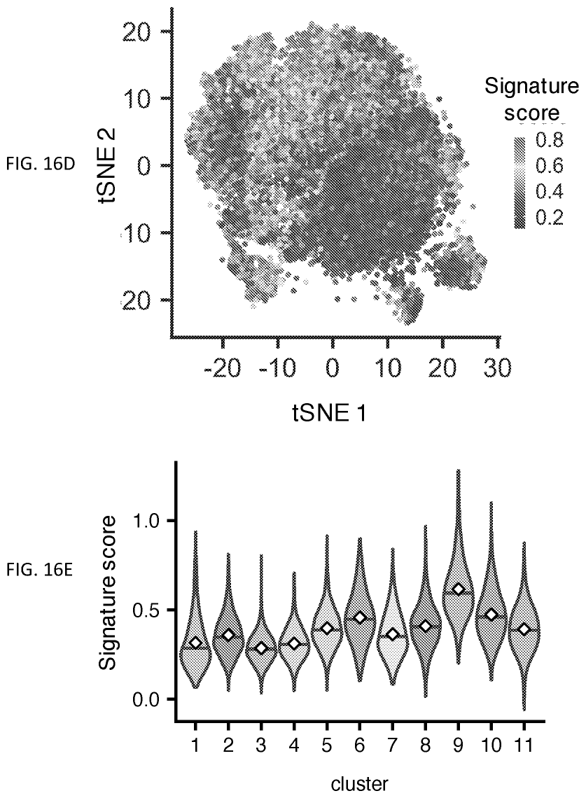

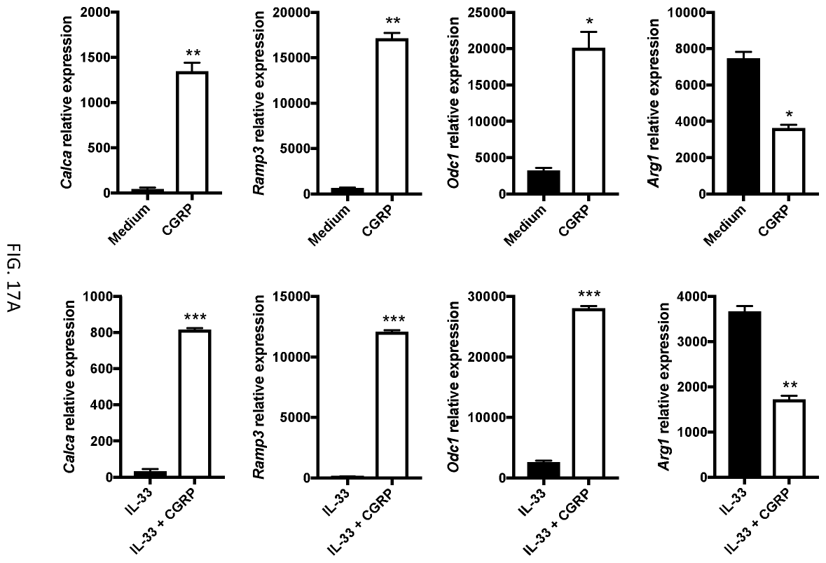

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different immune cells or immune cell (sub)populations (e.g., ILC2 cells), as well as comparing immune cells or immune cell (sub)populations with other immune cells or immune cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of genes may also be determined by comparing expression of genes in a population of cells or in single cells. In certain embodiments, expression of sets of genes is mutually exclusive in cells having a different cell state or subtype. In certain embodiments, a specific signature may have a certain set of genes upregulated or downregulated as compared to other genes in the signature (see, e.g., FIG. 16 A and C). For example, 1113 is upregulated in IL-33 induced inflammatory ILC2 cells, but Foxp3 is downregulated in the cell as compared to Il13 expression.

As discussed herein, differentially expressed genes/proteins, or differential epigenetic elements may be differentially expressed on a single cell level, or may be differentially expressed on a cell population level. Preferably, the differentially expressed genes/proteins or epigenetic elements as discussed herein, such as constituting the gene signatures as discussed herein, when as to the cell population level, refer to genes that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., ILC2) which can be distinguished or are uniquely identifiable and set apart from other cells of this cell type. The cell subpopulation may be phenotypically characterized, and is preferably characterized by the signature as discussed herein. A cell (sub)population as referred to herein may constitute of a (sub)population of cells of a particular cell type characterized by a specific cell state.

In certain embodiments, the gene signature is a biological program. As used herein the term “biological program” can be used interchangeably with “expression program” or “transcriptional program” and may refer to a set of genes that share a role in a biological function (e.g., an activation program, cell differentiation program, proliferation program). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

The invention further relates to various uses of the gene signatures, protein signature, and/or other genetic or epigenetic signature as defined herein, as well as various uses of the immune cells or immune cell (sub)populations as defined herein. Particular advantageous uses include methods for identifying agents capable of inducing or suppressing particular immune cell (sub)populations based on the gene signatures, protein signature, and/or other genetic or epigenetic signature as defined herein. The invention further relates to agents capable of inducing or suppressing particular immune cell (sub)populations based on the gene signatures, protein signature, and/or other genetic or epigenetic signature as defined herein, as well as their use for modulating, such as inducing or repressing, a particular gene signature, protein signature, and/or other genetic or epigenetic signature. In one embodiment, genes in one population of cells may be activated or suppressed in order to affect the cells of another population. In related aspects, modulating, such as inducing or repressing, a particular a particular gene signature, protein signature, and/or other genetic or epigenetic signature may modify overall immune composition, such as immune cell composition, such as immune cell subpopulation composition or distribution, or functionality.

The signature genes of the present invention were discovered by analysis of expression profiles of single-cells within different populations of lung resident innate lymphoid cells (ILC) (e.g., populations treated with alarmins, CGRP, NMU or combinations thereof), thus allowing the discovery of novel cell subtypes that were previously invisible in a population of cells within ILCs. The presence of subtypes may be determined by subtype specific signature genes. The presence of these specific cell types may be determined by applying the signature genes to bulk sequencing data in a patient. Not being bound by a theory, many cells that make up a microenvironment, whereby the cells communicate and affect each other in specific ways. As such, specific cell types within this microenvironment may express signature genes specific for this microenvironment. Not being bound by a theory the signature genes of the present invention may be microenvironment specific, such as their expression at a site of inflammation. The signature gene may indicate the presence of one particular cell type. In one embodiment, the expression may indicate the presence of inflammatory or protective cell types. Not being bound by a theory, a combination of cell subtypes in a subject may indicate an outcome.

In certain embodiments, a CGRP+IL-33 ILC2 gene signature (e.g., signature of differentially expressed genes between ILC2s treated with IL-33 and IL-33+CGRP; or IL-33 induced genes that can be modulated by CGRP) comprises one or more genes or polypeptides selected from the group consisting of. Sos1, Egfr, Tph1, P2ry1, Far1, Plin2, Alox5, Pparg, Ikzf1, Ier3, Rilpl2, Stap1, Gimap5, Odc1, Smox, Calca, Ramp3, Rora, Il7r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd4l, Jag1, Zfp36l1, Lmo4, Il13, Il6, Il4ra, Prdm1, Arg1, Zeb2, Srgap3, Ptger4, Pcsk1, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1 and Serpinb9. In certain embodiments, IL-33 induces an inflammatory gene signature and this signature can be reversed by treatment with CGRP.

In certain embodiments, treatment of ILC2s with CGRP alone provides for a CGRP gene signature comprising one or more genes selected from the group consisting of. Arg1, Ly6a, Stab1, Ptger4, Maf, Tph1, Traip, Kdm8, Birc5, Mki67, Crem, Fosl2, Odc1, Smox, Nr3c1, Rora, Lmo4, Ikzf3, Il7r, Il1rl1, Crlf2, Il17rb, Xbp1, Itk, Ccr4, Icos, Irf4, Pdcd1, Ctla2a, Fgl2, Gp49a, Nt5e, Tnfrsf9, Tnfrsf18, Lilrb4, Tnfaip3, Pde4d, Nmb, Calca, Ramp3, Serpinb9, Hif1a, Egln3. In certain embodiments, this signature can be used to monitor an immune response or monitor a response to a treatment (e.g., CGRP). In certain embodiments, a shift to higher expression of the signature indicates that the treatment is reducing an inflammatory response.

In certain embodiments, an ILC2 inflammatory gene signature comprises a) Anxa2; or b) Ltb; or c) one or more genes or polypeptides selected from the group consisting of Anxa1, Anxa2, Calca, Ccl1, Ccl5, Ccr2, Ccr7, Ccr8, Cd200r1, Cd3d, Cd47, Cd48, Cd81, Csf2, Ctla4, Fas, H2-Aa, H2-Ab1, H2-Q8, H2-T23, Il1r2, Il2rb, Il6, Lat, Lgals3, Lilrb4a, Ltb, Mif, Ms4a4b, Nmur1, Pdcd1, Pgk1, Ptger2, Ramp1, Sdc4, Sema4a, Sepp1, Stab2, Tff1, Tmem176a, Tnfrsf4, Tnfrsf8, Tnfsf8, Vsir, NMU, 2810417H13Rik, AA467197, Alox5, Atf4, Batf, Bcl2a1b, Blk, Btg1, Cox5b, Cox6c, Crip1, Dgat1, Dgat2, Dusp1, Ets1, Fos, Fosb, Furin, Gadd45b, Gsto1, Hint1, Ier2, Irf4, Klf3, Klf4, Lgmn, Lpcat2, Mcm3, Mt1, Myl6, Ndufa4, Nfkbia, Nfkbid, Nfkbiz, Nop56, Nr4a1, Prdx4, S100a4, S100a6, Serpinb6a, Snrpd3, Sptssa, Tph1, Vim, Zfp36 and Zfp36l1; or d) one or more genes or polypeptides selected from the group consisting of Anxa1, Anxa2, Calca, Ccl1, Ccl5, Ccr2, Ccr7, Ccr8, Cd200r1, Cd3d, Cd47, Cd48, Cd81, Csf2, Ctla4, Fas, H2-Aa, H2-Ab1, H2-Q8, H2-T23, Il1r2, Il2rb, Il6, Lat, Lgals3, Lilrb4a, Ltb, Mif, Ms4a4b, Nmur1, Pdcd1, Pgk1, Ptger2, Ramp1, Sdc4, Sema4a, Sepp1, Stab2, Tff1, Tmem176a, Tnfrsf4, Tnfrsf8, Tnfsf8, Vsir, NMU, 2810417H13Rik, AA467197, Alox5, Atf4, Batf, Bcl2a1b, Blk, Btg1, Cox5b, Cox6c, Crip1, Dgat1, Dgat2, Dusp1, Ets1, Fos, Fosb, Furin, Gadd45b, Gsto1, Hint1, Ier2, Irf4, Klf3, Klf4, Lgmn, Lpcat2, Mcm3, Mt1, My16, Ndufa4, Nfkbia, Nfkbid, Nfkbiz, Nop56, Nr4a1, Prdx4, S100a4, S100a6, Serpinb6a, Snrpd3, Sptssa, Tph1, Vim, Zfp36 and Zfp36l1; and one or more genes or polypeptides selected from the group consisting of Il5, Areg, IL-7Ra, CD90, Tbx21, Il1rl1, Il13, Klrg1, Arg1 and Ptprc; or e) one or more genes or polypeptides selected from the group consisting of Anxa2, Lgals3, Ctla4, Batf, Cd47, Tnfrsf8, AA467197, S100a6, Prdx4, Gsto1, Il1r2, Lgmn, Mt1, Tff1, Ccr7, Irf4, Il6, Tnfrsf4, H2-T23, Lilrb4a, Fas, Ets1, Ramp1, Nmru1, Dgat2, Calca, Ccl5, Btg1, Nr4a1, Klf3, Klf4, Csf2, Stab2, Sdc4, Ccr2, Fosb, Zfp36l1, Lpcat2 and Ltb; or f) one or more genes or polypeptides as in (e) and one or more genes or polypeptides selected from the group consisting of Il5 and Areg; or g) one or more genes or polypeptides as in (e) and one or more genes or polypeptides selected from the group consisting of IL-7Ra, CD90, Tbx21, Il1rl1, Il13, Klrg1, Arg1 and Ptprc; or h) one or more genes or polypeptides as in (e) and one or more genes or polypeptides selected from the group consisting of Il5, Areg, IL-7Ra, CD90, Tbx21, Il1rl1, Il13, Klrg1, Arg1 and Ptprc; or i) one or more genes or polypeptides selected from the group consisting of Anxa1, Anxa2, Calca, Ccl1, Ccl5, Ccr2, Ccr7, Ccr8, Cd200r1, Cd3d, Cd47, Cd48, Cd81, Csf2, Ctla4, Fas, H2-Aa, H2-Ab1, H2-Q8, H2-T23, Il1r2, Il2rb, Il6, Lat, Lgals3, Lilrb4a, Ltb, Mif, Ms4a4b, Nmur1, Pdcd1, Pgk1, Ptger2, Ramp1, Sdc4, Sema4a, Sepp1, Stab2, Tff1, Tmem176a, Tnfrsf4, Tnfrsf8, Tnfsf8, Vsir, NMU; or j) one or more genes or polypeptides as in (i) and one or more genes or polypeptides selected from the group consisting of Il5, Areg, Il13 and Klrg1; or k) one or more genes or polypeptides selected from the group consisting of Fosb, Btg2, Lpcat2, Sdc4, Csf2, Dgat2, Calca, Areg, Pim2, Zfp36l1, Nr4a1, Cd81, Ly6a, Lgmn, Il13, Il5, Klrg1, Batf, Pycard, Pdcd1, Lgals3, Anaxa2, Ctla4, Il1r2, Tox2, Tnfrsf8, Mt1, Tff1, Lilrb4a and H2-Ab1. The one or more genes or polypeptides selected from the group consisting of Anxa2, Lgals3, Ctla4, Batf, Cd47, Tnfrsf8, AA467197, S100a6, Prdx4, Gsto1, Il1r2, Lgmn, Mt1, Tff1, Ccr7, Irf4, 116, Tnfrsf4, H2-T23, Lilrb4a, Fas, Ets1, Ramp1, 115 and Areg may be upregulated. The one or more genes or polypeptides selected from the group consisting of Nmru1, Dgat2, Calca, Ccl5, Btg1, Nr4a1, Klf3, Klf4, Csf2, Stab2, Sdc4, Ccr2, Fosb, Zfp36l1, Lpcat2 and Ltb may be downregulated.

The one or more genes may be upregulated or downregulated in comparison to a reference sample or reference expression profile. The reference sample may be an untreated sample or a sample of non-inflammatory ILC2s. Detecting an innate lymphoid cell type 2 inflammatory response may be performed in a subject administered an allergic challenge.

In certain embodiments, the gene signature includes surface expressed proteins. In certain embodiments, surface proteins may be targeted for detection and isolation of cell types, or may be targeted therapeutically to modulate an immune response.

### Diagnostic and Detection Methods

The invention provides biomarkers (e.g., phenotype or cell type specific) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or +3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

In certain embodiments, diseases related to ILC2 responses as described further herein are diagnosed, prognosed, or monitored. For example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed b FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the oesophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

In certain embodiments, the method provides for treating a patient with CGRP, wherein the patient is suffering from a disease related to ILC2 inflammatory responses (e.g., allergy or asthma), the method comprising the steps of: determining whether the patient expresses a gene signature, biological program or marker gene as described herein: obtaining or having obtained a biological sample from the patient; and performing or having performed an assay as described herein on the biological sample to determine if the patient expresses the gene signature, biological program or marker gene; and if the patient has an ILC2 inflammatory gene signature, biological program or marker gene, then administering CGRP to the patient in an amount sufficient to shift the phenotype to a homeostatic or non-inflammatory phenotype, and if the patient does not have an ILC2 inflammatory gene signature, biological program or marker gene, then not administering CGRP to the patient, wherein a risk of having inflammatory symptoms is increased if the patient has an ILC2 inflammatory gene signature, biological program or marker gene.

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B. V. (1993) and Kricka, “Nonisotopic DNA Probe Techniques”, Academic Press, San Diego, Calif. (1992).

### Sequencing and Nucleic Acid Analysis

Various aspects and embodiments of the invention may involve analyzing gene signatures, protein signature, and/or other genetic or epigenetic signature based on single cell analyses (e.g. single cell RNA sequencing) or alternatively based on cell population analyses, as is defined herein elsewhere.

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Screening for Modulating Agents

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

### Genes and Polypeptides

All gene name symbols refer to the gene as commonly known in the art. The examples described herein that refer to the mouse gene names are to be understood to also encompasses human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. The signature as described herein may encompass any of the genes described herein. As used herein the terms “Calcitonin Gene-Related Peptide” and “CGRP” may refer to any of the mammalian, human, or mouse peptides α-CGRP, β-CGRP, their functional variants and fragments or any mammalian orthologues. CGRP also includes peptides having undergone post-translational modifications, such as peptides having covalent attachment of glycosyl groups, acetyl groups, phosphate groups, lipid groups, and the like. By functional variant of CGRP, it is herein referred to peptides which peptide sequence differ from the amino acid sequence CGRP, but that generally retains all the biological activity of CGRP. In certain embodiments, functional variants of CGRP are ligands binding to and activating the CGRP receptor. Functional variants may also include modified peptides, fusion proteins (e.g., fused to another protein, polypeptide or the like, such as an immunoglobulin or a fragment thereof), or peptides having non-natural amino acids. Functional variants may have an extended residence time in body fluids.

CGRP receptors have been described as heterodimeric molecules formed of the calcitonin receptor-like receptor (CRLR), linked to RAMP1 (CALCRL). RAMP1 is a transmembrane domain protein of the RAMP family, which further comprises RAMP2 and RAMP3. Several types of receptors are known that can be activated by CGRP: CGRP receptor (formed of CRLR and of RAMP1), AM₂ receptor (formed of CRLR and of RAMP3), and AMY₁ and AMY₃ receptors (formed of the calcitonin receptor and of RAMP1 and RAMP3, respectively). The CGRP receptors can therefore be distinguished from the AM₂, AMY₁ and AMY₃ receptors by the nature of the transmembrane domain of the RAMP family interacting with CRLR.

As used herein, “CGRP receptor”, refers to a protein receptor comprising the CRLR protein Ref NCBI: NP_005786.1), bound to the protein Receptor Activity Modifying Protein 1 (RAMP1) (Ref NCBI: NP_005846.1). Thus, CGRP receptors do not comprise the CRLR protein bound to RAMP2 or RAMP3.

In certain embodiments, a variant of CGRP has at least 80, 85, 90, 95, 99% of the biological activity of CGRP. In certain embodiments, a variant of α-CGRP has at least 80, 85, 90, 95, 99% of the biological activity of α-CGRP. In certain embodiments, a variant of β-CGRP has at least 80, 85, 90, 95, 99% of the biological activity of β-CGRP.

Preferably, a functional variant of α-CGRP has at least 85%, at least 90%, at least 91%, at least 92%, at least 93%, at least 94%, at least 95%, at least 96%, at least 96%, at least 97%, at least 98%, at least 99% sequence identity with α-CGRP. Preferably, a functional variant of β-CGRP has at least 85%, at least 90%, at least 91%, at least 92%, at least 93%, at least 94%, at least 95%, at least 96%, at least 96%, at least 97%, at least 98%, at least 99% sequence identity with β-CGRP.

As used herein, the term “functional fragments” refers to a specific peptide that has a biological activity of interest, which peptide sequence is a part of the peptide sequence of the reference peptide, and that can be of any length, provided the biological activity of peptide of reference is retained by said fragment.

The human peptide α-CGRP (UniProtKB/Swiss-Prot ref.: P06881.3) is encoded by the human gene CALCA (NCBI ref: NG 015960.1, NP_001029125.1) and has the sequence:

Ala-Cys-Asp-Thr-Ala-Thr-Cys-Val-Thr-His-Arg-Leu-Ala-Gly-Leu-Leu-Ser-Arg-Ser-Gly-Gly-Val-Val-Lys-Asn-Asn-Phe-Val-Pro-Thr-Asn-Val-Gly-Ser-Lys-Ala-Phe-NH2 (SEQ ID NO: 1).

The human peptide β-CGRP (UniProtKB/Swiss-Protref.: P10092.1) is encoded by the human gene CALCB (NCBI ref: NM_000728.4, NP_000719.1), and has the sequence:

Ala-Cys-Asn-Thr-Ala-Thr-Cys-Val-Thr-His-Arg-Leu-Ala-Gly-Leu-Leu-Ser-Arg-Ser-Gly-Gly-Met-Val-Lys-Ser-Asn-Phe-Val-Pro-Thr-Asn-Val-Gly-Ser-Lys-Ala-Phe-NH2 (SEQ ID NO: 2)

The gene name Areg or AREG may refer to the Amphiregulin gene or polypeptide according to NCBI Reference Sequence accession numbers NM_009704.4 or NM_001657.3. The gene name Calca or CALCA may refer to the Calcitonin/calcitonin-related polypeptide, alpha gene or polypeptide according to NCBI Reference Sequence accession numbers NM_001033954.3, NM 007587.2, NM_001033952.2, NM_001033953.2 or NM_001741.2. The gene name Ramp1 or RAMP1 may refer to the Receptor (calcitonin) activity modifying protein 1 gene or polypeptide according to NCBI Reference Sequence accession numbers NM_016894.3, NM_001168392.1, or NM_005855.3.

### Modulation and Modulating Agents

In certain embodiments, ILC2 cells, ILC2 gene signatures, ILC2 immune responses (e.g., inflammatory responses, homeostasis) are modulated. As used herein, “modulating” or “to modulate” generally means either reducing or inhibiting the expression or activity of, or alternatively increasing the expression or activity of a target or antigen (e.g., CGRP). In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, at least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen, such as CGRP. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein, such as CGRP. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

In certain embodiments, the agent modulates CGRP signaling. In certain embodiments, the agent is an agonist or antagonist of CGRP receptor activity. As used herein, the term “agonist of the CGRP receptor”, refers to a compound that binds to a CGRP receptor and activates said CGRP receptor (see, e.g., US20160106813A1).

In certain embodiments, administration of CGRP provokes migraine attacks due to its vasodilation properties, and are associated with dilation of both the middle meningeal artery (MMA), a major artery that supplies blood to a membrane (dura) that envelops the brain, and the middle cerebral artery (MCA) (see, e.g., Silberstein et al., Fremanezumab for the Preventive Treatment of Chronic Migraine, N Engl J Med 2017; 377:2113-22). Several approaches are possible to diminish the potential side-effects of the compounds of the invention. These side-effects can be diminished by following a specific treatment scheme, more precisely by making sure that the consecutive administrations are separated by enough time without CGRP and/or agonist of the CGRP receptor treatment. In a particular embodiment, the consecutive administrations of CGRP and/or agonist of the CGRP receptor are separated by at least 1 day, preferably 2 days, yet preferably 5 days.

The composition of the invention can also advantageously be formulated in order to release CGRP and/or agonist of the CGRP receptor in the subject in a timely controlled fashion. In a particular embodiment, the composition of the invention is formulated for controlled release of CGRP and/or agonist of the CGRP receptor.

In certain embodiments, the agent is capable of inhibiting CGRP receptor or blocking CGRP receptor interaction with CGRP. Such agents may also be referred to as CGRP receptor antagonists. In certain embodiments, CGRP receptor or CGRP expression is inhibited, e.g., by a DNA targeting agent (e.g., CRISPR system, TALE, Zinc finger protein) or a RNA targeting agent (e.g., inhibitory nucleic acid molecules). In some embodiments, CGRP receptor activity is inhibited. Such inhibition includes, e.g., reducing the expression of its ligand, CGRP, or by blocking the interaction of CGRP receptor with CGRP. In certain embodiments, the antagonist is an antibody or fragment thereof. In certain embodiments, the antibody is specific for CGRP or CGRP receptor.

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents (e.g., CGRP) and allow for particular agents to be capable of crossing the blood-brain barrier. Thus, in one embodiment, PEGylating CGRP or the CGRP receptor agonists or antagonists improve the pharmacokinetics and pharmacodynamics of the CGRP receptor agonists or antagonists.

In regards to peptide PEGylation methods, reference is made to Lu et al., Int. J. Pept. Protein Res.43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif 12: 45-52 (1998). Polyethylene glycol or PEG is meant to encompass any of the forms of PEG that have been used to derivatize other proteins, including, but not limited to, mono-(C1-10) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide (e.g., CGRP) via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH2)2SH) residues at any position in a peptide. In certain embodiments, the CGRP receptor agonists described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. In certain embodiments, CGRP receptor agonists are PEGylated through the side chains of a cysteine residue added to the N-terminal amino acid.

In exemplary embodiments, the PEG molecule(s) may be covalently attached to an amide group in the C-terminus of a peptide, such as in the CGRP receptor agonist. In preferred embodiments, there is at least one PEG molecule covalently attached to the CGRP receptor agonist. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., neuromedin U receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents (e.g., CGRP, or CGRP agonist or antagonists) include a protecting group covalently joined to the N-terminal amino group. In exemplary embodiments, a protecting group covalently joined to the N-terminal amino group of the CGRP receptor agonists reduces the reactivity of the amino terminus under in vivo conditions. Amino protecting groups include —C1-10 alkyl, —C1-10 substituted alkyl, —C2-10 alkenyl, —C2-10 substituted alkenyl, aryl, —C1-6 alkyl aryl, —C(O)—(CH2)1-6-COOH, C(O)—C1-6 alkyl, —C(O)-aryl, C(O)—O—C1-6 alkyl, or C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents (e.g., CGRP, or CGRP receptor agonists or antagonists) wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents (e.g., CGRP receptor agonists or antagonists) are modified by PEGylation, cholesterylation, or palmitoylation. The modification can be to any amino acid residue. In preferred embodiments, the modification is to the N-terminal amino acid of the agent (e.g., CGRP receptor agonist or antagonists), either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent (e.g., CGRP receptor agonist or antagonist) comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention (e.g., CGRP receptor agonists or antagonists) is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

In certain embodiments, combinations of targets are modulated (e.g., CGRP and one or more targets related to a gene signature gene). In certain embodiments, an agent against one of the targets in a combination may already be known or used clinically. In certain embodiments, targeting the combination may require less of the agent as compared to the current standard of care and provide for less toxicity and improved treatment.

### Glucocorticoids

In certain embodiments, the method further comprises administering a glucocorticoid, wherein the glucocorticoid is co-administered or administered after the therapeutically effective amount of α-CGRP or derivative thereof, or the α-CGRP receptor agonist. As described further herein, CGRP induces expression of the glucocorticoid receptor. Thus, CGRP treatment could enhance sensitivity to glucocorticoid activity in suppressing inflammation. Glucocorticoids (GCs) are a class of corticosteroids, which are a class of steroid hormones. Glucocorticoids are corticosteroids that bind to the glucocorticoid receptor (GR) that is present in almost every vertebrate animal cell. The name glucocorticoid (glucose+cortex+steroid) is composed from its role in regulation of glucose metabolism, synthesis in the adrenal cortex, and its steroidal structure. The glucocorticoid receptor (GR, or GCR) also known as NR3C1 (nuclear receptor subfamily 3, group C, member 1) is the receptor to which cortisol and other glucocorticoids bind. Administering a glucocorticoid in response to upregulation of NR3C1 by CGRP may reduce an ILC2 inflammatory response or maintain homeostasis. Glucocorticoids have previously been described for use in treating asthma by regulating ILC2s (see, e.g., Yu et al., ILC2 frequency and activity are inhibited by glucocorticoid treatment via STAT pathway in patients with asthma, Allergy. 2018 September; 73(9): 1860-1870). Example glucocorticoids applicable to the present invention include, but are not limited to Cortisol (hydrocortisone), Cortisone, Prednisone, Prednisolone, Methylprednisolone, Dexamethasone, Betamethasone, Triamcinolone, Fludrocortisone acetate, Deoxycorticosterone acetate and budesonide.

### Immune Checkpoints

Immune checkpoints are regulators of the immune system. These pathways are crucial for self-tolerance, which prevents the immune system from attacking cells indiscriminately. Modulating immune checkpoint activity in response to upregulation by CGRP may reduce an ILC2 inflammatory response or maintain homeostasis. The check point blockade therapy may be an inhibitor of any check point protein described herein. The checkpoint blockade therapy may comprise anti-TIM3, anti-CTLA4, anti-PD-L1, anti-PD1, or combinations thereof. Anti-PD1 antibodies are disclosed in U.S. Pat. No. 8,735,553. Anti-CTLA4 antibodies are disclosed in U.S. Pat. Nos. 9,327,014; 9,320,811; and 9,062,111.

Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab). Immune checkpoint agonists may activate the checkpoint signaling, for example, by binding to the checkpoint protein. The agonists may include a ligand. PD-1 agonist antibodies that mimic PD-1 ligand have been described (see, e.g., US20170088618A1; WO2018053405A1). Such agonist antibodies against any receptor described herein are applicable to the present invention. In certain embodiments, the invention comprises administering one or more agonists or antagonists of PD-1 or TIM-3. In certain embodiments, the agonist or antagonist is an antibody, small molecule or ligand.

### CD39

In certain embodiments, the invention comprises administering one or more agonists or antagonists of CD39, wherein the one or more agonists or antagonists are co-administered or administered after the therapeutically effective amount of α-CGRP or derivative thereof; or the α-CGRP receptor agonist. As used herein, the term “CD39” has its general meaning in the art and refers to the CD39 protein also named as ectonucleoside triphosphate diphosphohydrolase-1 (ENTPD1). CD39 is an ectoenzyme that hydrolases ATP/UTP and ADP/UDP to the respective nucleosides such as AMP. Modulating CD39 activity in response to upregulation by CGRP may reduce an ILC2 inflammatory response or maintain homeostasis. Accordingly, the term “CD39 inhibitor” refers to a compound that inhibits the activity or expression of CD39. In some embodiments, the CD39 inhibitor is an antibody having specificity for CD39. In certain embodiments, the CD39 inhibitor is a small molecule. CD39 activity modulators are well known in the art. For example, 6-N,N-Diethyl-d-β-γ-dibromomethylene adenosine triphosphate (ARL 67156) (Levesque et al (2007) Br, J. Pharmacol, 152: 141-150; Crack et al. (1959) Br. J. Pharmacol. 114: 475-481; Kennedy et al. (1996) Semtn. Neurosci. 8: 195-199) and 8-thiobutyladenosine 5′-triphosphate (8-Bu-S-ATP) are small molecule CD39 inhibitors (Gendron et al. (2000) J Med Chem. 43:2239-2247). Other small molecule CD39 inhibitors, such as polyoxymetate-1 (POM-1) and α,β-methylene ADP (APCP), are also well known in the art (see, U.S. 2010/204182 and US2013/0123345; U.S. Pat. No. 6,617,439). In addition, nucleic acid and antibody inhibitors of CD39 are also well known in the art (see, e.g., US20130273062A1).

### GITR

In certain embodiments, the invention comprises administering one or more agonists or antagonists of GITR, wherein the one or more agonists or antagonists are co-administered or administered after the therapeutically effective amount of α-CGRP or derivative thereof; or the α-CGRP receptor agonist. Glucocorticoid-induced tumor necrosis factor receptor (GITR/TNFRSF18/CD357/AITR) is a surface receptor molecule that has been shown to be involved in inhibiting the suppressive activity of T-regulatory cells and extending the survival of T-effector cells. Modulating GITR activity in response to upregulation by CGRP may reduce an ILC2 inflammatory response or maintain homeostasis. GITR modulating antibodies and recombinant GITRL (GITR ligand) have been described and tested in preclinical tumor models (see, e.g., Knee et al., Rationale for anti-GITR cancer immunotherapy. Eur J Cancer. 2016 November; 67:1-10).

### LILRB4

In certain embodiments, the invention comprises administering one or more agonists or antagonists of LILRB4, wherein the one or more agonists or antagonists are co-administered or administered after the therapeutically effective amount of α-CGRP or derivative thereof; or the α-CGRP receptor agonist. Leukocyte immunoglobulin-like receptor subfamily B member 4 is a protein that in humans is encoded by the LILRB4 gene. This gene is a member of the leukocyte immunoglobulin-like receptor (LIR) family, which is found in a gene cluster at chromosomal region 19q13.4. The encoded protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. The receptor can also function in antigen capture and presentation. It is thought to control inflammatory responses and cytotoxicity to help focus the immune response and limit autoreactivity. Multiple transcript variants encoding different isoforms have been found for this gene. LILRB4 has been shown to interact with PTPN6. Modulating LILRB4 activity in response to upregulation by CGRP may reduce an ILC2 inflammatory response or maintain homeostasis. Agonists of LILRB4 have been described (see, e.g., WO2013181438A2). By the term “LILRB4 agonist” is meant an agent that specifically binds to LILRB4 protein and activates LILRB4 signaling pathways in a mammalian cell. Antagonists of LILRB4 have been described (see, e.g., US20180086829A1).

### Antibodies

The term “antibody” (e.g., anti-CGRP or anti-CGRP receptor antibody) is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, VHH and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG-IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (1° F.n3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, VH and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having VH and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind CGRP receptor or CGRP and inhibit the ability to suppress an ILC class 2 inflammatory response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibit CGRP receptor or CGRP. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target locus of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

In certain example embodiments, the CRISPR effector protein may be delivered using a nucleic acid molecule encoding the CRISPR effector protein. The nucleic acid molecule encoding a CRISPR effector protein, may advantageously be a codon optimized CRISPR effector protein. An example of a codon optimized sequence, is in this instance a sequence optimized for expression in eukaryote, e.g., humans (i.e. being optimized for expression in humans), or for another eukaryote, animal or mammal as herein discussed; see, e.g., SaCas9 human codon optimized sequence in WO 2014/093622 (PCT/US2013/074667). Whilst this is preferred, it will be appreciated that other examples are possible and codon optimization for a host species other than human, or for codon optimization for specific organs is known. In some embodiments, an enzyme coding sequence encoding a CRISPR effector protein is a codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a Cas correspond to the most frequently used codon for a particular amino acid.

In certain embodiments, the methods as described herein may comprise providing a Cas transgenic cell in which one or more nucleic acids encoding one or more guide RNAs are provided or introduced operably connected in the cell with a regulatory element comprising a promoter of one or more gene of interest. As used herein, the term “Cas transgenic cell” refers to a cell, such as a eukaryotic cell, in which a Cas gene has been genomically integrated. The nature, type, or origin of the cell are not particularly limiting according to the present invention. Also the way the Cas transgene is introduced in the cell may vary and can be any method as is known in the art. In certain embodiments, the Cas transgenic cell is obtained by introducing the Cas transgene in an isolated cell. In certain other embodiments, the Cas transgenic cell is obtained by isolating cells from a Cas transgenic organism. By means of example, and without limitation, the Cas transgenic cell as referred to herein may be derived from a Cas transgenic eukaryote, such as a Cas knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas transgene can further comprise a Lox-Stop-polyA-Lox(LSL) cassette thereby rendering Cas expression inducible by Cre recombinase. Alternatively, the Cas transgenic cell may be obtained by introducing the Cas transgene in an isolated cell. Delivery systems for transgenes are well known in the art. By means of example, the Cas transgene may be delivered in for instance eukaryotic cell by means of vector (e.g., AAV, adenovirus, lentivirus) and/or particle and/or nanoparticle delivery, as also described herein elsewhere.

It will be understood by the skilled person that the cell, such as the Cas transgenic cell, as referred to herein may comprise further genomic alterations besides having an integrated Cas gene or the mutations arising from the sequence specific action of Cas when complexed with RNA capable of guiding Cas to a target locus.

In certain aspects the invention involves vectors, e.g. for delivering or introducing in a cell Cas and/or RNA capable of guiding Cas to a target locus (i.e. guide RNA), but also for propagating these components (e.g. in prokaryotic cells). A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle and if the target is present in the delivery vesicle the CRISPR effector is activated and a detectable signal generated.

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector, is to express an array of promoter-RNAs separated by cleavable sequences in the intron of a coding sequence or gene; and, in this instance it is advantageous to use a polymerase II promoter, which can have increased expression and enable the transcription of long RNA in a tissue specific manner. (see, e.g., nar.oxfordjournals.org/content/34/7/e53.short and nature.com/mt/journal/v16/n9/abs/mt2008144a.html). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters-especially as to the numbers of RNAs or guides discussed herein, without any undue experimentation.

The guide RNA(s) encoding sequences and/or Cas encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may but need not be structurally related, or are only partially structurally related. An “orthologue” of a protein as used herein is a protein of a different species which performs the same or a similar function as the protein it is an orthologue of Orthologous proteins may but need not be structurally related, or are only partially structurally related.

### Guide Molecules

The methods described herein may be used to screen inhibition of CRISPR systems employing different types of guide molecules. As used herein, the term “guide sequence” and “guide molecule” in the context of a CRISPR-Cas system, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that a RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, it is of interest to reduce the susceptibility of the guide molecule to RNA cleavage, such as to cleavage by Cas13. Accordingly, in particular embodiments, the guide molecule is adjusted to avoid cleavage by Cas13 or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′ phosphorothioate (MS), S-constrained ethyl(cEt), or 2′-O-methyl 3′ thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucletides and/or nucleotide analogs in a region that binds to Cas13. In an embodiment of the invention, deoxyribonucleotides and/or nucleotide analogs are incorporated in engineered guide structures, such as, without limitation, stem-loop regions, and the seed region. For Cas13 guide, in certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554).

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (Ψ), N1-methylpseudouridine (me1Ψ), 5-methoxyuridine(5moU), inosine, 7-methylguanosine, 2′-O-methyl 3′phosphorothioate (MS), S-constrained ethyl(cEt), phosphorothioate (PS), or 2′-O-methyl 3′thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 to 10 nucleotides in the 3′-terminus are chemically modified. Such chemical modifications at the 3′-terminus of the Cas13 CrRNA may improve Cas13 activity. In a specific embodiment, 1, 2, 3, 4, 5, 6, 7, 8, 9 or 10 nucleotides in the 3′-terminus are replaced with 2′-fluoro analogues. In a specific embodiment, 1, 2, 3, 4, 5, 6, 7, 8, 9 or 10 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the modified loop comprises 3, 4, or 5 nucleotides. In certain embodiments, the loop comprises the sequence of UCUU, UUUU, UAUU, or UGUU.

In some embodiments, the guide molecule forms a stemloop with a separate non-covalently linked sequence, which can be DNA or RNA. In particular embodiments, the sequences forming the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) from the guide sequence. In a particular embodiment the seed sequence (i.e. the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g. an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e. corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNAses or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a basepair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM. In the embodiments of the present invention where the CRISPR-Cas protein is a Cas13 protein, the complementary sequence of the target sequence is downstream or 3′ of the PAM or upstream or 5′ of the PAM. The precise sequence and length requirements for the PAM differ depending on the Cas13 protein used, but PAMs are typically 2-5 base pair sequences adjacent the protospacer (that is, the target sequence). Examples of the natural PAM sequences for different Cas13 orthologues are provided herein below and the skilled person will be able to identify further PAM sequences for use with a given Cas13 protein.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously.

In particular embodiment, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. The invention accordingly comprehends an guide molecule that responds to normal or pathological physiological conditions, including without limitation pH, hypoxia, O₂ concentration, temperature, protein concentration, enzymatic concentration, lipid structure, light exposure, mechanical disruption (e.g. ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

The invention contemplates energy sources such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm². In a preferred embodiment, a stimulation paradigm of as low as 0.25 sec every 15 sec should result in maximal activation.

The chemical or energy sensitive guide may undergo a conformational change upon induction by the binding of a chemical source or by the energy allowing it act as a guide and have the Cas13 CRISPR-Cas system or complex function. The invention can involve applying the chemical source or energy so as to have the guide function and the Cas13 CRISPR-Cas system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans; 4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the Cas13 CRISPR-Cas complex or system, and the binding will induce the change of sub-cellular localization of the polypeptide, leading to the entire polypeptide entering the nucleus of cells. Once inside the nucleus, the guide protein and the other components of the Cas13 CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc, as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100·mu·s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz’ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp.136-142. Another form of focused ultrasound is high intensity focused ultrasound (HIFU) which is reviewed by Moussatov et al in Ultrasonics (1998) Vol. 36, No. 8, pp.893-900 and TranHuuHue et al in Acustica (1997) Vol. 83, No. 6, pp.1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e. a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

### CRISPR RNA-Targeting Effector Proteins

In one example embodiment, the CRISPR system effector protein is an RNA-targeting effector protein. In certain embodiments, the CRISPR system effector protein is a Type VI CRISPR system targeting RNA (e.g., Cas13a, Cas13b, Cas13c or Cas13d). Example RNA-targeting effector proteins include Cas13b and C2c2 (now known as Cas13a). It will be understood that the term “C2c2” herein is used interchangeably with “Cas13a”. “C2c2” is now referred to as “Cas13a”, and the terms are used interchangeably herein unless indicated otherwise. As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 has been described in Abudayyeh et al. (2016) “C2c2 is a single-component programmable RNA-guided RNA-targeting CRISPR effector”; Science; DOI: 10.1126/science.aaf5573; and Shmakov et al. (2015) “Discovery and Functional Characterization of Diverse Class 2 CRISPR-Cas Systems”, Molecular Cell, DOI: dx.doi.org/10.1016/j.molcel.2015.10.008; which are incorporated herein in their entirety by reference. Cas13b has been described in Smargon et al. (2017) “Cas13b Is a Type VI-B CRISPR-Associated RNA-Guided RNases Differentially Regulated by Accessory Proteins Csx27 and Csx28,” Molecular Cell. 65, 1-13; dx.doi.org/10.1016/j.molcel.2016.12.023, which is incorporated herein in its entirety by reference.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent Application entitled “Novel Type VI CRISPR Orthologs and Systems,” labeled as attorney docket number 47627-05-2133 and filed on Apr. 12, 2017.

In certain other example embodiments, the CRISPR system effector protein is a C2c2 nuclease (also referred to as Cas13a). The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

In certain embodiments, the C2c2 effector protein is from an organism of a genus selected from the group consisting of: Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Campylobacter, and Lachnospira, or the C2c2 effector protein is an organism selected from the group consisting of: Leptotrichia shahii, Leptotrichia. wadei, Listeria seeligeri, Clostridium aminophilum, Carnobacterium gallinarum, Paludibacter propionicigenes, Listeria weihenstephanensis, or the C2c2 effector protein is a L. wadei F0279 or L. wadei F0279 (Lw2) C2C2 effector protein. In another embodiment, the one or more guide RNAs are designed to detect a single nucleotide polymorphism, splice variant of a transcript, or a frameshift mutation in a target RNA or DNA.

In certain example embodiments, the RNA-targeting effector protein is a Type VI-B effector protein, such as Cas13b and Group 29 or Group 30 proteins. In certain example embodiments, the RNA-targeting effector protein comprises one or more HEPN domains. In certain example embodiments, the RNA-targeting effector protein comprises a C-terminal HEPN domain, a N-terminal HEPN domain, or both. Regarding example Type VI-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/US2016/058302 entitled “Novel CRISPR Enzymes and Systems”, and filed Oct. 21, 2016, and Smargon et al. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In particular embodiments, the Cas13b enzyme is derived from Bergeyella zoohelcum.

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYLL. WYL1 is a single WYL-domain protein associated primarily with Ruminococcus.

In other example embodiments, the Type VI RNA-targeting Cas enzyme is Cas13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Cas13 RNA Editing

In one aspect, the invention provides a method of modifying or editing a target transcript in a eukaryotic cell. In some embodiments, the method comprises allowing a CRISPR-Cas effector module complex to bind to the target polynucleotide to effect RNA base editing, wherein the CRISPR-Cas effector module complex comprises a Cas effector module complexed with a guide sequence hybridized to a target sequence within said target polynucleotide, wherein said guide sequence is linked to a direct repeat sequence. In some embodiments, the Cas effector module comprises a catalytically inactive CRISPR-Cas protein. In some embodiments, the guide sequence is designed to introduce one or more mismatches to the RNA/RNA duplex formed between the target sequence and the guide sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present application relates to modifying a target RNA sequence of interest (see, e.g, Cox et al., Science. 2017 Nov. 24; 358(6366):1019-1027). Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and not have to enter the nucleus, making them easier to deliver.

A further aspect of the invention relates to the method and composition as envisaged herein for use in prophylactic or therapeutic treatment, preferably wherein said target locus of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors. In particular embodiments, the invention thus comprises compositions for use in therapy. This implies that the methods can be performed in vivo, ex vivo or in vitro. In particular embodiments, when the target is a human or animal target, the method is carried out ex vivo or in vitro.

A further aspect of the invention relates to the method as envisaged herein for use in prophylactic or therapeutic treatment, preferably wherein said target of interest is within a human or animal and to methods of modifying an Adenine or Cytidine in a target RNA sequence of interest, comprising delivering to said target RNA, the composition as described herein. In particular embodiments, the CRISPR system and the adenosine deaminase, or catalytic domain thereof, are delivered as one or more polynucleotide molecules, as a ribonucleoprotein complex, optionally via particles, vesicles, or one or more viral vectors.

In one aspect, the invention provides a method of generating a eukaryotic cell comprising a modified or edited gene. In some embodiments, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide, wherein the guide sequence may be designed to introduce one or more mismatches between the RNA/RNA duplex formed between the guide sequence and the target sequence. In particular embodiments, the mismatch is an A-C mismatch. In some embodiments, the Cas effector may associate with one or more functional domains (e.g. via fusion protein or suitable linkers). In some embodiments, the effector domain comprises one or more cytidine or adenosine deaminases that mediate endogenous editing of via hydrolytic deamination. In particular embodiments, the effector domain comprises the adenosine deaminase acting on RNA (ADAR) family of enzymes. In particular embodiments, the adenosine deaminase protein or catalytic domain thereof capable of deaminating adenosine or cytidine in RNA or is an RNA specific adenosine deaminase and/or is a bacterial, human, cephalopod, or Drosophila adenosine deaminase protein or catalytic domain thereof, preferably TadA, more preferably ADAR, optionally huADAR, optionally (hu)ADAR1 or (hu)ADAR2, preferably huADAR2 or catalytic domain thereof.

The present invention may also use a Cas12 CRISPR enzyme. Cas12 enzymes include Cas12a (Cpf1), Cas12b (C2c1), and Cas12c (C2c3), described further herein.

A further aspect relates to an isolated cell obtained or obtainable from the methods described herein comprising the composition described herein or progeny of said modified cell, preferably wherein said cell comprises a hypoxanthine or a guanine in replace of said Adenine in said target RNA of interest compared to a corresponding cell not subjected to the method. In particular embodiments, the cell is a eukaryotic cell, preferably a human or non-human animal cell, optionally a therapeutic T cell or an antibody-producing B-cell.

In some embodiments, the modified cell is a therapeutic T cell, such as a T cell suitable for adoptive cell transfer therapies (e.g., CAR-T therapies). The modification may result in one or more desirable traits in the therapeutic T cell, as described further herein.

The invention further relates to a method for cell therapy, comprising administering to a patient in need thereof the modified cell described herein, wherein the presence of the modified cell remedies a disease in the patient.

The present invention may be further illustrated and extended based on aspects of CRISPR-Cas development and use as set forth in the following articles and particularly as relates to delivery of a CRISPR protein complex and uses of an RNA guided endonuclease in cells and organisms:
Multiplex genome engineering using CRISPR-Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR-Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 August 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013);
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR-Cas9 system, Wang T, Wei J J, Sabatini D M, Lander E S., Science. January 3; 343(6166): 80-84. doi:10.1126/science.1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR-Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
BCL11A enhancer dissection by Cas9-mediated in situ saturating mutagenesis, Canver et al., Nature 527(7577):192-7 (Nov. 12, 2015) doi: 10.1038/nature15521. Epub 2015 Sep. 16.
Cpf1 Is a Single RNA-Guided Endonuclease of a Class 2 CRISPR-Cas System, Zetsche et al., Cell 163, 759-71 (Sep. 25, 2015).
Discovery and Functional Characterization of Diverse Class 2 CRISPR-Cas Systems, Shmakov et al., Molecular Cell, 60(3), 385-397 doi: 10.1016/j.molcel.2015.10.008 Epub Oct. 22, 2015.
Rationally engineered Cas9 nucleases with improved specificity, Slaymaker et al., Science 2016 Jan. 1 351(6268): 84-88 doi: 10.1126/science.aad5227. Epub 2015 Dec. 1.
Gao et al, “Engineered Cpf1 Enzymes with Altered PAM Specificities,” bioRxiv 091611; doi: http://dx.doi.org/10.1101/091611 (Dec. 4, 2016).
Cox et al., “RNA editing with CRISPR-Cas13,” Science. 2017 Nov. 24; 358(6366):1019-1027. doi: 10.1126/science.aaq0180. Epub 2017 Oct. 25.
Gaudelli et al. “Programmable base editing of A-T to G-C in genomic DNA without DNA cleavage” Nature 464(551); 464-471 (2017).

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR-Cas system for the one-step generation of mice carrying mutations in multiple genes which were traditionally generated in multiple steps by sequential recombination in embryonic stem cells and/or time-consuming intercrossing of mice with a single mutation. The CRISPR-Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and guide RNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR-Cas9 knockout and nucleotide preference at the cleavage site. The authors also found that the sequence preference for CRISPRi/a is substantially different from that for CRISPR-Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Canver et al. (2015) demonstrated a CRISPR-Cas9-based functional investigation of non-coding genomic elements. The authors developed pooled CRISPR-Cas9 guide RNA libraries to perform in situ saturating mutagenesis of the human and mouse BCL11A enhancers which revealed critical features of the enhancers.
Zetsche et al. (2015) reported characterization of Cpf1, a class 2 CRISPR nuclease from Francisella novicida U112 having features distinct from Cas9. Cpf1 is a single RNA-guided endonuclease lacking tracrRNA, utilizes a T-rich protospacer-adjacent motif, and cleaves DNA via a staggered DNA double-stranded break.
Shmakov et al. (2015) reported three distinct Class 2 CRISPR-Cas systems. Two system CRISPR enzymes (C2c1 and C2c3) contain RuvC-like endonuclease domains distantly related to Cpf1. Unlike Cpf1, C2c1 depends on both crRNA and tracrRNA for DNA cleavage. The third enzyme (C2c2) contains two predicted HEPN RNase domains and is tracrRNA independent.
Slaymaker et al (2016) reported the use of structure-guided protein engineering to improve the specificity of Streptococcus pyogenes Cas9 (SpCas9). The authors developed “enhanced specificity” SpCas9 (eSpCas9) variants which maintained robust on-target cleavage with reduced off-target effects.
Cox et al., (2017) reported the use of catalytically inactive Cas13 (dCas13) to direct adenosine-to-inosine deaminase activity by ADAR2 (adenosine deaminase acting on RNA type 2) to transcripts in mammalian cells. The system, referred to as RNA Editing for Programmable A to I Replacement (REPAIR), has no strict sequence constraints and can be used to edit full-length transcripts. The authors further engineered the system to create a high-specificity variant and minimized the system to facilitate viral delivery.

The methods and tools provided herein are may be designed for use with or Cas13, a type II nuclease that does not make use of tracrRNA. Orthologs of Cas13 have been identified in different bacterial species as described herein. Further type II nucleases with similar properties can be identified using methods described in the art (Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

Also, Harrington et al. “Programmed DNA destruction by miniature CRISPR-Cas14 enzymes” Science 2018 doi:10/1126/science.aav4293, relates to Cas14.

With respect to general information on CRISPR/Cas Systems, components thereof, and delivery of such components, including methods, materials, delivery vehicles, vectors, particles, and making and using thereof, including as to amounts and formulations, as well as CRISPR-Cas-expressing eukaryotic cells, CRISPR-Cas expressing eukaryotes, such as a mouse, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, and 8,945,839; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); 14/054,414 European Patent Applications EP 2 771 468 (EP13818570.7), EP 2 764 103 (EP13824232.6), and EP 2 784 162 (EP14170383.5); and PCT Patent Publications WO2014/093661 (PCT/US2013/074743), WO2014/093694 (PCT/US2013/074790), WO2014/093595 (PCT/US2013/074611), WO2014/093718 (PCT/US2013/074825), WO2014/093709 (PCT/US2013/074812), WO2014/093622 (PCT/US2013/074667), WO2014/093635 (PCT/US2013/074691), WO2014/093655 (PCT/US2013/074736), WO2014/093712 (PCT/US2013/074819), WO2014/093701 (PCT/US2013/074800), WO2014/018423 (PCT/US2013/051418), WO2014/204723 (PCT/US2014/041790), WO2014/204724 (PCT/US2014/041800), WO2014/204725 (PCT/US2014/041803), WO2014/204726 (PCT/US2014/041804), WO2014/204727 (PCT/US2014/041806), WO2014/204728 (PCT/US2014/041808), WO2014/204729 (PCT/US2014/041809), WO2015/089351 (PCT/US2014/069897), WO2015/089354 (PCT/US2014/069902), WO2015/089364 (PCT/US2014/069925), WO2015/089427 (PCT/US2014/070068), WO2015/089462 (PCT/US2014/070127), WO2015/089419 (PCT/US2014/070057), WO2015/089465 (PCT/US2014/070135), WO2015/089486 (PCT/US2014/070175), WO2015/058052 (PCT/US2014/061077), WO2015/070083 (PCT/US2014/064663), WO2015/089354 (PCT/US2014/069902), WO2015/089351 (PCT/US2014/069897), WO2015/089364 (PCT/US2014/069925), WO2015/089427 (PCT/US2014/070068), WO2015/089473 (PCT/US2014/070152), WO2015/089486 (PCT/US2014/070175), WO2016/049258 (PCT/US2015/051830), WO2016/094867 (PCT/US2015/065385), WO2016/094872 (PCT/US2015/065393), WO2016/094874 (PCT/US2015/065396), WO2016/106244 (PCT/US2015/067177).

Mention is also made of U.S. application 62/180,709, 17-Jun.-15, PROTECTED GUIDE RNAS (PGRNAS); U.S. application 62/091,455, filed, 12-Dec.-14, PROTECTED GUIDE RNAS (PGRNAS); U.S. application 62/096,708, 24-Dec.-14, PROTECTED GUIDE RNAS (PGRNAS); U.S. applications 62/091,462, 12-Dec.-14, 62/096,324, 23-Dec.-14, 62/180,681, 17-Jun.-2015, and 62/237,496, 5 Oct. 2015, DEAD GUIDES FOR CRISPR TRANSCRIPTION FACTORS; U.S. application 62/091,456, 12-Dec.-14 and 62/180,692, 17 Jun. 2015, ESCORTED AND FUNCTIONALIZED GUIDES FOR CRISPR-CAS SYSTEMS; U.S. application 62/091,461, 12-Dec.-14, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19-Dec.-14, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24-Dec.-14, ENGINEERING OF SYSTEMS, METHODS AND OPTIMIZED ENZYME AND GUIDE SCAFFOLDS FOR SEQUENCE MANIPULATION; U.S. application 62/098,059, 30-Dec.-14, 62/181,641, 18 Jun. 2015, and 62/181,667, 18 Jun. 2015, RNA-TARGETING SYSTEM; U.S. application 62/096,656, 24-Dec.-14 and 62/181,151, 17 Jun. 2015, CRISPR HAVING OR ASSOCIATED WITH DESTABILIZATION DOMAINS; U.S. application 62/096,697, 24-Dec.-14, CRISPR HAVING OR ASSOCIATED WITH AAV; U.S. application 62/098,158, 30-Dec.-14, ENGINEERED CRISPR COMPLEX INSERTIONAL TARGETING SYSTEMS; U.S. application 62/151,052, 22-Apr.-15, CELLULAR TARGETING FOR EXTRACELLULAR EXOSOMAL REPORTING; U.S. application 62/054,490, 24-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12-Feb.-14, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25-Sep.-14, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4-Dec.-14, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24-Sep.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23-Oct.-14, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24-Sep.-14 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24-Sep.-14, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25-Sep.-14, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25-Sep.-14, MULTIFUNCTIONAL-CRISPR COMPLEXES AND/OR OPTIMIZED ENZYME LINKED FUNCTIONAL-CRISPR COMPLEXES; U.S. application 62/087,475, 4-Dec.-14 and 62/181,690, 18 Jun. 2015, FUNCTIONAL SCREENING WITH OPTIMIZED FUNCTIONAL CRISPR-CAS SYSTEMS; U.S. application 62/055,487, 25-Sep.-14, FUNCTIONAL SCREENING WITH OPTIMIZED FUNCTIONAL CRISPR-CAS SYSTEMS; U.S. application 62/087,546, 4-Dec.-14 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30-Dec.-14, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19-Aug.-2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24-Sep.-2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12-Feb.-2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8).

In particular embodiments, the ribonucleoprotein is delivered by way of a polypeptide-based shuttle agent as described in WO2016161516. WO2016161516 describes efficient transduction of polypeptide cargos using synthetic peptides comprising an endosome leakage domain (ELD) operably linked to a cell penetrating domain (CPD), to a histidine-rich domain and a CPD. Similarly these polypeptides can be used for the delivery of CRISPR-effector based RNPs in eukaryotic cells.

### Tale Systems

As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In advantageous embodiments of the invention, the methods provided herein use isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

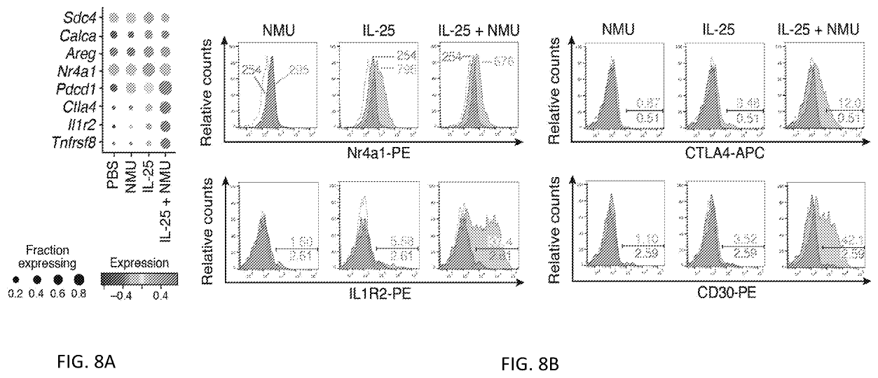

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ. I.D. No. 3)
M D P I R S R T P S P A R E L L S G P Q P D
G V Q P T A D R G V S P P A G G P L D G L P
A R R T M S R T R L P S P P A P S P A F S A
D S F S D L L R Q F D P S L F N T S L F D S
L P P F G A H H T E A A T G E W D E V Q S G
L R A A D A P P P T M R V A V T A A R P P R
A K P A P R R R A A Q P S D A S P A A Q V D
L R T L G Y S Q Q Q Q E K I K P K V R S T V
A Q H H E A L V G H G F T H A H I V A L S Q
H P A A L G T V A V K Y Q D M I A A L P E A
T H E A I V G V G K Q W S G A R A L E A L L
T V A G E L R G P P L Q L D T G Q L L K I A
K R G G V T A V E A V H A W R N A L T G A P
L N

An exemplary amino acid sequence of a C-terminal capping region is:

(SEQ. I.D. No. 4)
R P A L E S I V A Q L S R P D P A L A A L T
N D H L V A L A C L G G R P A L D A V K K G
L P H A P A L I K R T N R R I P E R T S H R
V A D H A Q V V R V L G F F Q C H S H P A Q
A F D D A M T Q F G M S R H G L L Q L F R R
V G V T E L E A R S G T L P P A S Q R W D R
I L Q A S G M K R A K P S P T S T Q T P D Q
A S L H A F A D S L E R D L D A P S P M H E
G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In advantageous embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4× domain or a Kruppel-associated box (KRAB) or fragments of the KRAB domain. In some embodiments, the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### ZN-Finger Nucleases

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

As disclosed herein editing can be made by way of meganucleases, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

Diseases It will be understood by the skilled person that treating as referred to herein encompasses enhancing treatment, or improving treatment efficacy. Treatment may include inhibition of an inflammatory response, tumor regression as well as inhibition of tumor growth, metastasis or tumor cell proliferation, or inhibition or reduction of otherwise deleterious effects associated with the tumor.

Efficaciousness of treatment is determined in association with any known method for diagnosing or treating the particular disease. The invention comprehends a treatment method comprising any one of the methods or uses herein discussed.

The phrase “therapeutically effective amount” as used herein refers to a sufficient amount of a drug, agent, or compound to provide a desired therapeutic effect.

As used herein “patient” refers to any human being receiving or who may receive medical treatment and is used interchangeably herein with the term “subject”.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

The disclosure provides CGRP or derivatives thereof, or an agonist of the CGRP receptor for treating disease. A skilled person can readily determine diseases that can be treated by reducing an ILC2 inflammatory response (e.g., an IL-33 mediated disease or disorder). ILC2 cells and ILC2 inflammatory responses have been associated with allergic asthma, therapy resistant-asthma, steroid-resistant severe allergic airway inflammation, systemic steroid-dependent severe eosinophilic asthma, chronic rhino-sinusitis (CRS), atopic dermatitis, food allergies, persistence of chronic airway inflammation, and primary eosinophilic gastrointestinal disorders (EGIDs), including but not limited to eosinophilic esophagitis (EoE), eosinophilic gastritis, eosinophilic gastroenteritis, and eosinophilic colitis (see, e.g., Van Rijt et al., Type 2 innate lymphoid cells: at the cross-roads in allergic asthma, Seminars in Immunopathology July 2016, Volume 38, Issue 4, pp 483-496; Rivas et al., IL-4 production by group 2 innate lymphoid cells promotes food allergy by blocking regulatory T-cell function, J Allergy Clin Immunol. 2016 September; 138(3):801-811.e9; and Morita, Hideaki et al. Innate lymphoid cells in allergic and nonallergic inflammation, Journal of Allergy and Clinical Immunology, Volume 138, Issue 5, 1253-1264). Asthma is characterized by recurrent episodes of wheezing, shortness of breath, chest tightness, and coughing. Sputum may be produced from the lung by coughing but is often hard to bring up. During recovery from an attack, it may appear pus-like due to high levels of eosinophils. Symptoms are usually worse at night and in the early morning or in response to exercise or cold air. Some people with asthma rarely experience symptoms, usually in response to triggers, whereas others may have marked and persistent symptoms. CRS is characterized by inflammation of the mucosal surfaces of the nose and para-nasal sinuses, and it often coexists with allergic asthma. Atopic dermatitis is a chronic inflammatory skin disease that is characterized by eosinophilic infiltration and high serum IgE levels. Similar to allergic asthma and CRS, atopic dermatitis has been associated with increased expression of TSLP, IL-25, and IL-33 in the skin. Primary eosinophilic gastrointestinal disorders (EGIDs), including eosinophilic esophagitis (EoE), eosinophilic gastritis, eosinophilic gastroenteritis, and eosinophilic colitis, are disorders that exhibit eosinophil-rich inflammation in the gastrointestinal tract in the absence of known causes for eosinophilia such as parasite infection and drug reaction. Not being bound by a theory, corticosteroids suppress TH2 cells, but not ILC2s and cannot be used to modulate ILC2 inflammatory responses. Applicants have discovered factors that balance homeostatic and pathological pro-inflammatory ILC2 responses. In certain embodiments, modulation of these factors, as described herein, may be used to treat the diseases described. In preferred embodiments, CGRP signaling is modulated.

In certain embodiments, an IL-33 mediated disease or disorder that can be treated by reducing an ILC2 inflammatory response may be any inflammatory disease or disorder such as, but not limited to, asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

The asthma may be allergic asthma, non-allergic asthma, severe refractory asthma, asthma exacerbations, viral-induced asthma or viral-induced asthma exacerbations, steroid resistant asthma, steroid sensitive asthma, eosinophilic asthma or non-eosinophilic asthma and other related disorders characterized by airway inflammation or airway hyperresponsiveness (AHR).

The COPD may be a disease or disorder associated in part with, or caused by, cigarette smoke, air pollution, occupational chemicals, allergy or airway hyperresponsiveness.

The allergy may be associated with foods, pollen, mold, dust mites, animals, or animal dander.

The IBD may be ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

The arthritis may be selected from the group consisting of osteoarthritis, rheumatoid arthritis and psoriatic arthritis.

The disclosure also provides methods for enhancing an ILC2 type response and treating disease. In certain embodiments, tissue inflammatory ILC2s are switched to activated, tissue protective ILC2s. ILC2 cells have been shown to promote an eosinophil cytotoxic response, antitumor response and metastasis suppression (Ikutani et al., Identification of Innate IL-5-Producing Cells and Their Role in Lung Eosinophil Regulation and Antitumor Immunity, J Immunol 2012; 188:703-713). Specifically, innate IL-5-producing cells were increased in response to tumor invasion, and their regulation of eosinophils was critical to suppress tumor metastasis. Thus, in one embodiment induction of an ILC2 inflammatory response may be used in treating cancer. In other embodiments, the cancer is resistant to therapies targeting the adaptive immune system (see e.g., Rooney et al., Molecular and genetic properties of tumors associated with local immune cytolytic activity, Cell. 2015 Jan. 15; 160(1-2): 48-61). In one embodiment, modulation of CGRP signaling is used for inducing an inflammatory immune response state for the treatment of a subpopulation of tumor cells that are linked to resistance to targeted therapies and progressive tumor growth. Not being bound by a theory, in cases where tumors are resistant to therapies targeting the adaptive immune system, treatments targeting the innate immune system may be therapeutically effective in treating the tumor.

The cancer may include, without limitation, liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma.

The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocelluar carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein (e.g., CGRP receptor agonists or antagonists) may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

In another embodiment, the delivery system may be an administration device. As used herein, an administration device can be any pharmaceutically acceptable device adapted to deliver a composition of the invention (e.g., to a subject's nose). A nasal administration device can be a metered administration device (metered volume, metered dose, or metered-weight) or a continuous (or substantially continuous) aerosol-producing device. Suitable nasal administration devices also include devices that can be adapted or modified for nasal administration. In some embodiments, the nasally administered dose can be absorbed into the bloodstream of a subject.

A metered nasal administration device delivers a fixed (metered) volume or amount (dose) of a nasal composition upon each actuation. Exemplary metered dose devices for nasal administration include, by way of example and without limitation, an atomizer, sprayer, dropper, squeeze tube, squeeze-type spray bottle, pipette, ampule, nasal cannula, metered dose device, nasal spray inhaler, breath actuated bi-directional delivery device, pump spray, pre-compression metered dose spray pump, monospray pump, bispray pump, and pressurized metered dose device. The administration device can be a single-dose disposable device, single-dose reusable device, multi-dose disposable device or multi-dose reusable device. The compositions of the invention can be used with any known metered administration device.

A continuous aerosol-producing device delivers a mist or aerosol comprising droplet of a nasal composition dispersed in a continuous gas phase (such as air). A nebulizer, pulsating aerosol nebulizer, and a nasalcontinuous positive air pressure device are exemplary of such a device. Suitable nebulizers include, by way of example and without limitation, an air driven jet nebulizer, ultrasonic nebulizer, capillary nebulizer, electromagnetic nebulizer, pulsating membrane nebulizer, pulsating plate (disc) nebulizer, pulsating/vibrating mesh nebulizer, vibrating plate nebulizer, a nebulizer comprising a vibration generator and an aqueous chamber, a nebulizer comprising a nozzle array, and nebulizers that extrude a liquid formulation through a self-contained nozzle array.

In certain embodiments, the device can be any commercially available administration devices that are used or can be adapted for nasal administration of a composition of the invention (see, e.g., US patent publication US20090312724A1).

The amount of the agents (e.g., CGRP receptor agonist) which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5-500 micrograms (μg) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies (e.g., anti-CGRP receptor or anti-CGRP antibodies) are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g. phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1-1000 μg, more preferably 2-500, even more preferably 5-50, most preferably 10-20 μg per unit dosage form. In certain embodiments, infusion of antibodies of the present invention may range from 10-500 mg/m².

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

In another aspect, provided is an administration device, pharmaceutical pack or kit, comprising one or more containers filled with one or more of the ingredients of the pharmaceutical compositions, such as CGRP receptor agonists or antagonists (e.g., α-CGRP), and/or additional therapeutic agents.

The invention is further described in the following examples, which do not limit the scope of the invention described in the claims.

### EXAMPLES

### Example 1—IL-33-Mediated Activation and Expansion of Pro-Inflammatory ILC2s is Regulated by the Neuropeptide CGRP

To better define the transcriptional landscape of lung-resident ILCs, Applicants analyzed more than 24,187 high quality, droplet-based scRNA-seq profiles of IL-7Rα+ CD90+ Lineage-lung-resident ILCs at both steady state and after in vivo activation with either IL-25 or IL-33. Applicants complemented the droplet based survey with an analysis of single ILCs using a modified version of the SMART-Seq2 protocol, optimized for performance on small cells, including T cells and ILCs (Methods). Using this alternative method, Applicants analyzed 606 high-quality lung-resident ILC scRNA-seq profiles from mice treated with IL-33, IL-25, or PBS. The majority of these cells were classified as ILC2 by their gene signature score, consistent with the droplet-based atlas analysis (see, Wallrapp, et al., The neuropeptide NMU amplifies ILC2-driven allergic lung inflammation, Nature. 2017 Sep. 21; 549(7672):351-356. doi: 10.1038/nature24029. Epub 2017 Sep. 13).

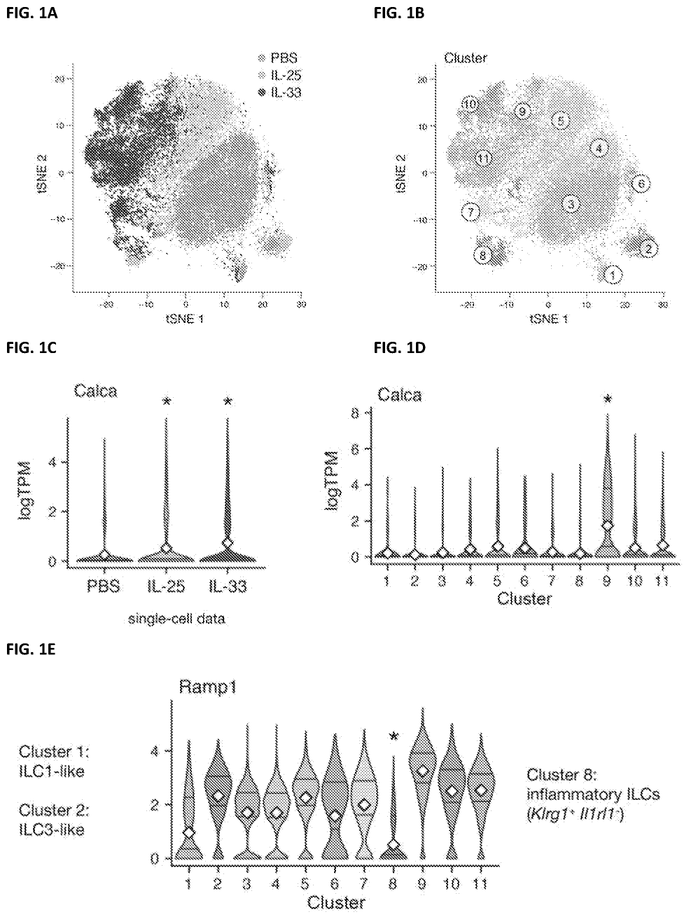

To identify novel neuropeptides that regulate ILC function, Applicants looked in the single-cell ILC data set for the expression of both neuropeptides and neuropeptide receptors, particularly those that are differentially expressed between homeostatic and inflammatory lung-derived ILCs. One of the neuropeptides that was expressed in ILCs from all treatment conditions and was upregulated after alarmin treatment was CGRP (Calca) (FIG. 1c). Applicants noted that ILCs expressed CGRP and that CGRP expression was increased by alarmin stimulation, most markedly in one specific cluster of ILCs (FIG. 1a-d). Moreover, ILCs also expressed the two subunits that make up the receptor for CGRP, Calcrl (calcitonin receptor like receptor), which is shared with several other neuropeptides, and Ramp1 (Receptor activity modifying protein 1), which specifically binds CGRP. Moreover, Ramp1 was highly expressed on most ILC clusters, with the exception of cluster 8, which consists of a pro-inflammatory KLRG1^{hi} ST2⁻ population (see, e.g., Huang et al., Nature Immunology Volume 16 Number 2 Feb. 2015) (FIG. 1e). This suggests that ILCs can both produce and respond to CGRP.

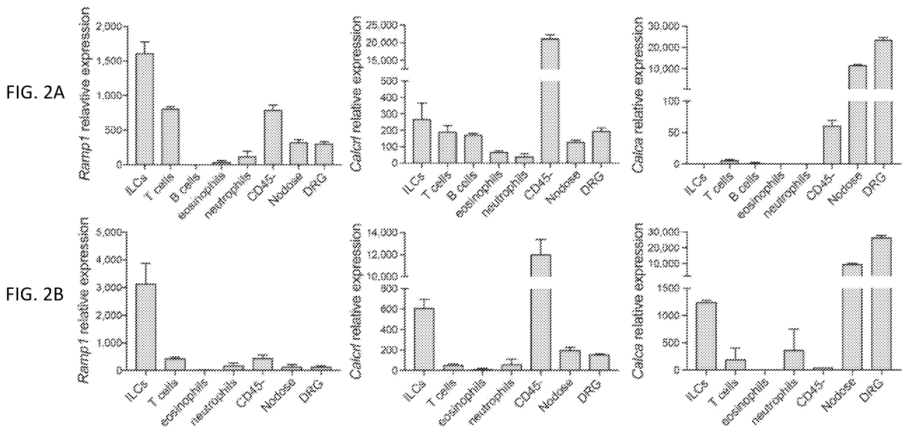

Sensory neurons, such as nociceptors, also produce CGRP, and several publications report an inhibitory function of CGRP on immune cells, such as macrophages and dendritic cells. Thus, Applicants hypothesized that CGRP may be produced by ILCs to attenuate immune responses, thereby preventing exaggerated tissue inflammation and damage. Applicants isolated ILCs, T cells, B cells, eosinophils, neutrophils, CD45− cells and lung innervating sensory neurons (nodose and dorsal root ganglion) from naïve mice and mice with airway inflammation and analyzed the expression of Ramp1 and Calcrl as well as Calca by qPCR. While Calcrl was expressed by most cell types, Ramp1 expression was highest in ILCs, particularly during airway inflammation (FIG. 2a,b) suggesting that ILCs may be particularly responsive to CGRP. CGRP (Calca) was highly expressed in CD45− cells and sensory neurons during steady-state, however, during airway inflammation it was also highly expressed in ILCs, suggesting that ILCs produce CGRP during inflammation. Thus, CGRP can potentially act in an autocrine manner to inhibit inflammatory ILC responses.

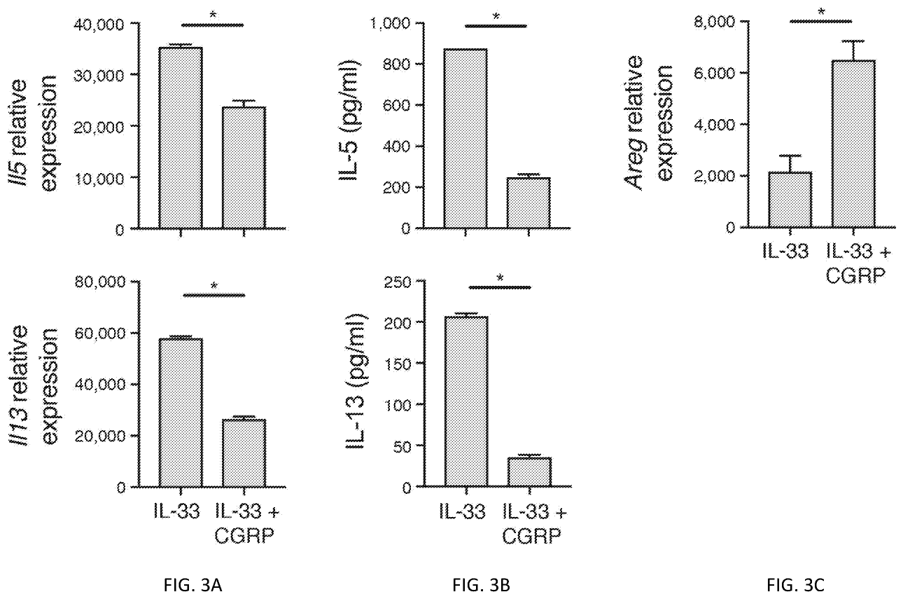

To investigate the role of CGRP specifically on ILCs, Applicants isolated lung ILCs and cultured them in vitro in the presence of IL-33 or IL-33+CGRP. CGRP significantly reduced IL-5 and IL-13 at both the RNA and protein level in ILCs, suggesting that it has an inhibitory effect on inflammatory type 2 cytokine production in ILCs at both the RNA and protein levels (FIG. 3a,b). Interestingly, after IL-33 stimulation, the addition of CGRP increased ILC expression of Areg (FIG. 3c), which encodes the epidermal growth factor amphiregulin. Amphiregulin produced by ILC2s is important for tissue integrity and repair after influenza virus infection (REF). Thus, CGRP appears to both limit inflammatory cytokine production by ILCs and also enhance expression of amphiregulin to facilitate tissue repair. Applicants have preliminarily observed the opposite effect in CGRP deficient ILCs.

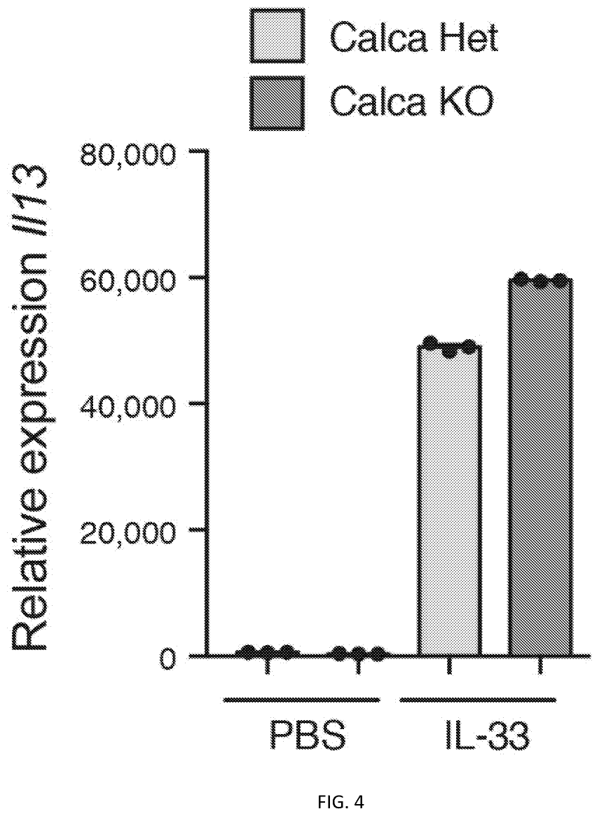

ILCs from CGRP Het and CGRP KO mice were cultured in vitro with PBS or IL-33 (FIG. 4). After three days, the ILCs expressed more IL-13 in the absence of endogenous CGRP, suggesting that ILC-derived CGRP may represent an autoregulatory loop. Thus, CGRP limits inflammatory cytokine production by ILCs, potentially in part via an autocrine mechanism.

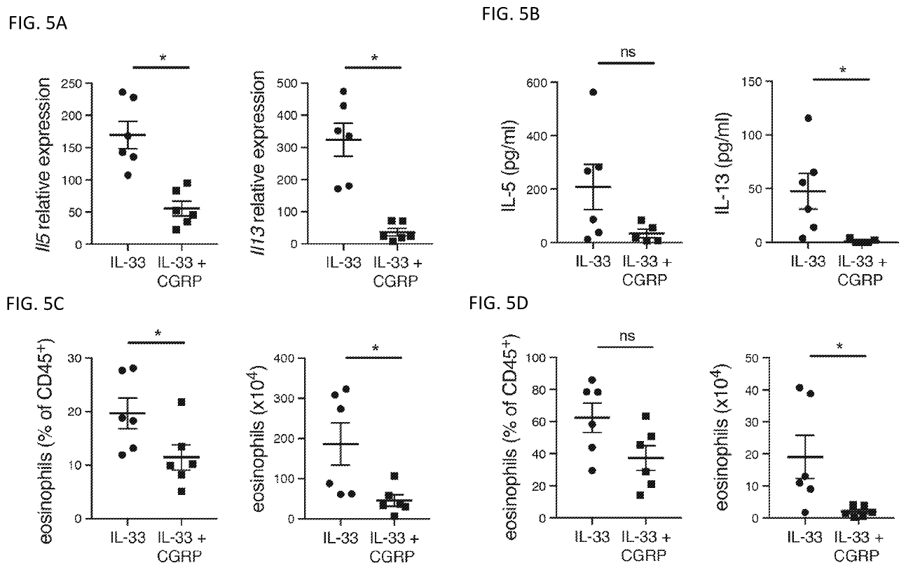

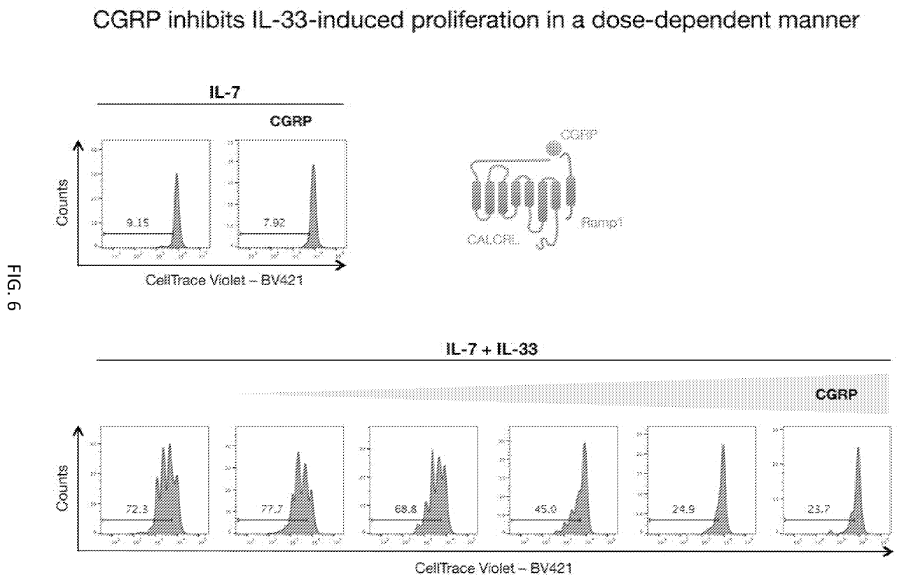

To study the role of CGRP in vivo, mice were treated intranasally with either IL-33 alone or in conjunction with CGRP (IL-33+CGRP) for three consecutive days. IL-33+CGRP treatment reduced 115 and 1113 RNA expression in lung tissue, as well as IL-5 and IL-13 protein levels in BALF compared to IL-33 alone (FIG. 5a,b). The frequency of eosinophils was significantly reduced in the lungs and BALF of mice receiving IL-33+CGRP, as were eosinophil numbers in the lung (FIG. 5c,d), indicating that CGRP limits IL-33-induced airway inflammation in vivo. This is potentially mediated via CGRP-mediated regulation of ILCs, although the widespread expression of CGRP and its receptor mean that other cellular pathways may also be involved.

Previous studies have shown proinflammatory responses, such as attenuation of antigen-induced airway hyperresponsiveness in CGRP-deficient mice (Aoki-Nagase et al. 2002 Am J Physiol Lung Cell Mol Physiol. 2002 November; 283(5):L963-70), deficiency of RAMP1 attenuates antigen-induced airway hyperresponsiveness in mice (Li et al. 2014 PLoS ONE 9(7): e102356), and Ramp1-deficient mice have increased colitis severity after DSS treatment (CGRP suppresses pro-inflammatory cytokine production by CD11c+DCs) (de Jong et al. 2015 Mucosal Immunology Volume 8 Number 3).

To study the role of CGRP in IL-33 induced proliferation Applicants labeled ILCs with CellTrace Violet for flow cytometric analysis (FIG. 6). Applicants observed that CGRP inhibits IL-33-induced proliferation in a dose-dependent manner.

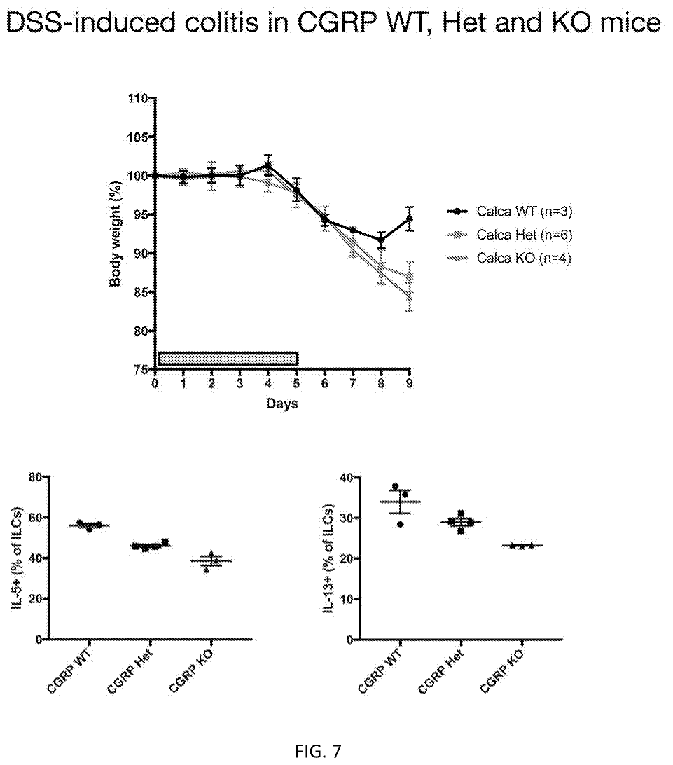

To study the role of CGRP in colitis, Applicants analyzed DSS-induced colitis in CGRP WT, Het and KO mice (FIG. 7). WT mice had decreased colitis compared to CGRP heterozygous and KO mice. Applicants also determined the percentage of ILCs positive for IL-5 and IL-13 present in the mice.

Together, these data support the idea that IL-33-mediated activation and expansion of pro-inflammatory ILC2s is regulated by the neuropeptide CGRP, which suppresses pro-inflammatory cytokine production by ILC2s, thus inhibiting the development of allergic inflammation.

Applicants can also look in other disease models of allergic inflammation such as food allergy. Using the CGRP KO mice described herein Applicants can further investigate the role of CGRP in vivo in allergic airway and intestinal inflammation, and allow for the use of adoptive transfer experiments to examine the ILC-specific role of CGRP in vivo.

Applicants can further study the mechanism by which the neuropeptide CGRP regulates ILC2 function and allergic lung inflammation. The initial studies show that in addition to Nmur1, ILC2s express both Ramp1 and Calcrl, which together form the receptor for CGRP, another neuropeptide, and exposure to CGRP inhibits type 2 cytokine production by ILC2s in response to IL-33. Since IL-33 alone strongly activates ILC2s and results in tissue inflammation, induction of CGRP receptor expression might be one mechanism by which to inhibit ILC2-mediated allergic inflammation. However, the mechanisms by which CGRP inhibits pro-inflammatory ILC2s are unclear.

CGRP is predominantly produced by central and peripheral neurons, including nociceptors. However, during allergic lung inflammation, it is not clear what cell types produce CGRP or when it is produced. Applicants hypothesize that CGRP may be produced in the lung later during inflammation to promote resolution of allergic inflammation in the lung. ILCs are in close proximity to neurons in the lung tissue (Wallrapp, et al.) and in the gut (15, 16) and could thereby receive CGRP signals directly from the neurons. However, a number of cell types that regulate development of lung inflammation, including ILCs and neuroendocrine cells, also produce CGRP (30, 38). Applicants therefore hypothesize that multiple cell types may produce CGRP to prevent exaggerated allergic immune responses. Applicants isolated ILCs, T cells, B cells, eosinophils, and neutrophils from both naive and allergen challenged mice, and analyzed expression of Ramp1, Calcrl, and Calca (CGRP) by qPCR. While Applicants found almost no CGRP expression in immune cells during steady-state (FIG. 2a), Applicants surprisingly found high levels of CGRP expression in ILCs as compared to other immune cells during airway inflammation (FIG. 2b). Using the droplet-based single-cell transcriptional data set of over 24,000 ILCs from PBS-, IL-25- and IL-33-treated mice, Applicants confirmed that CGRP (Calca) is upregulated in ILCs after alarmin stimulation, compared to PBS (FIG. 1c), further confirming that indeed CGRP is also produced by activated ILC2s. ILCs from one of the clusters preferentially expressed CGRP (FIG. 1b,d). This finding suggests that a particular subset of ILCs may produce CGRP to regulate allergic inflammation.

Applicants hypothesized that CGRP is upregulated later during inflammation to prevent exaggerated pro-inflammatory ILC2 responses. The preliminary experiments showed that CGRP expression is upregulated in a number of immune cell populations, and particularly in ILCs, following allergen challenge (FIG. 2b). To directly address this issue, Applicants will sort immune and non-immune cells from the lungs on days 0, 1, 2, 3, 5, and 8 following intranasal IL-33 administration, and analyze Calca expression by qPCR, as compared to PBS treated controls. Applicants will also isolate cells and analyze Calca expression in ILCs and lung parenchymal cells on days 9, 10, 12, and 15 following the acute HDM challenge protocol. In order to characterize the spatial relationships of ILCs and CGRP-producing cell populations, Applicants will analyze lung sections by immunofluorescence microscopy at different time points and stain for ILCs (KLRG1+ CD3−) along with CGRP (and other cell type specific markers as necessary) to confirm expression of CGRP by ILCs. Since there is always a risk in immunohistology that the molecule is not produced but present in the cell due to receptor-mediated uptake, Applicants will determine Calca expression in sorted populations of lung cells. Finally, in addition to analyzing CGRP expression immediately upon isolation, Applicants will also assess the expression of CGRP in cultured ILCs after IL-33 stimulation, on both the RNA and protein level, using qPCR and a CGRP Enzyme Immunoassay (EIA) (Phoenix Pharmaceuticals).

The observations that alarmins induce ILCs that express CGRP in vivo and that CGRP inhibits cytokine production by ILCs in vitro suggest that ILC-intrinsic expression of CGRP might mediate an autocrine inhibitory feedback loop. To test this hypothesis, Applicants will culture ILCs from WT and CGRP−/− mice in vitro with IL-33 and analyze expression of Il5, Il13, and Areg by qPCR and ELISA after 24 or 72 hours of stimulation. Applicants will determine whether loss of CGRP induces an exaggerated pro-inflammatory phenotype with increased IL-5 and IL-13 production. This would suggest that CGRP produced by ILC2s themselves may inhibit the development of a proinflammatory phenotype via an autocrine feed-back loop, thereby controlling chronic inflammation.

In addition to ILC2s, Th2 cells are critical for the induction of allergic inflammation. This raises the question of whether Th2 cells are also susceptible to CGRP-mediated inhibition. CGRP produced during the resolution phase of allergic lung inflammation may suppress development of allergies by acting on both ILC2s and Th2 cells. To address this, Applicants will generate in vitro differentiated Th2 cells, and analyze expression of Ramp1 and Calcrl, the two key receptor chains for CGRP. Applicants will also treat Th2 cells with graded doses of CGRP in vitro, and analyze type 2 cytokine expression (IL-4, IL-5, and IL-13) by ELISA and intracellular cytokine staining. In addition, Applicants will isolate mRNA from the CGRP-treated Th2 cells and perform multiplex Nanostring nCounter gene expression analysis using a custom Nanostring codeset that includes cytokines and transcription factors specific to each of the T helper subsets.

Although the CGRP-receptor is expressed on multiple cell types, Applicants will determine whether CGRP-deficient mice develop exaggerated allergic lung inflammation following allergen or alarmin challenge. Applicants will challenge CGRP−/− mice and wild type littermates with HDM, IL-25, IL-33, or IL-25+NMU. The animals will be tested for all the parameters of allergic lung inflammation. If CGRP generally suppresses airway inflammation, one would expect that CGRP-deficient mice would have exaggerated responses to these stimuli. However, since the receptor for CGRP is expressed on multiple cell types and may mediate diverse downstream signaling events, it is possible that airway inflammation in CGRP−/− mice will not be exaggerated. Indeed, some studies suggest that CGRP−/− and RAMP-1−/− mice have attenuated allergen-induced airway hyper-responsiveness, suggesting CGRP may in fact enhance allergic lung inflammation (Aoki-Nagase et al. 2002; and Li et al. 2014). However, the role of CGRP following challenge with alarmins such as IL-33 or IL-25+NMU, which primarily act on ILC2s, or after HDM challenge, remains to be determined, and it is possible that there are differential effects of CGRP depending on the model of allergic lung inflammation used.

In addition to testing the development of allergic inflammation in CGRP−/− mice, Applicants will also test the direct impact of CGRP administration on the development of allergic lung inflammation. For this purpose, Applicants will induce allergic lung inflammation by three different protocols: HDM, IL-33 and IL-25+NMU. Applicants will simultaneously administer three different doses of CGRP intranasally on days 0, 1, and 2 following IL-33 or IL-25+NMU challenge, or on days 7, 8, and 9 after HDM challenge, to determine whether co-administration of CGRP impacts various phenotypes of allergic lung inflammation. Notably, Applicants will evaluate whether CGRP antagonizes NMU induced allergic lung inflammation (in IL-25+NMU induced disease), as the preliminary data suggests that these two neuropeptides have differing effects on ILC function.

CGRP inhibits pro-inflammatory ILCs in vitro, however since the CGRP receptor is expressed by many cell types, the reduced airway inflammation in CGRP treated mice may be due to effects on other immune cells. To specifically investigate the function of CGRP on ILCs during airway inflammation, Applicants will adoptively transfer WT ILCs into Calcrl−/−/Ramp1−/− mice. In preliminary experiments, Applicants will establish that the transferred WT ILCs migrate to lung tissue and survive over time. After engraftment, the recipient mice will receive IL-33 or house dust mite (HDM) to induce airway inflammation, together with either CGRP or PBS as a control. Since only the transferred ILCs will express Ramp1 and can thus respond to CGRP, Applicants can use this experimental set-up to investigate the ILC-specific impact of CGRP administration. Severity of airway inflammation will be assessed by different physiological readouts. To investigate how CGRP inhibits ILC function, Applicants will analyze lung-resident ILCs ex vivo by flow cytometry for the expression of cytokines, activation markers, and the proliferation marker ki67. If Applicants find that ILCs are the critical cell type that mediates CGRP's inhibitory effect, Applicants will isolate ILCs and assess their transcriptome by RNA-seq. These experiments will allow us to determine whether the reduced airway inflammation that Applicants see in CGRP-treated mice is primarily mediated by ILCs and how CGRP signaling changes the transcriptional state of ILCs in vivo.

Applicants have found that ILCs themselves produce CGRP, suggesting that CGRP produced by ILCs may regulate allergic inflammation as a feed-back inhibitory loop. To investigate whether ILC-derived CGRP inhibits airway inflammation Applicants will adoptively transfer WT or Calca-deficient ILCs into RAG2−/− IL-2Rγ−/− mice, which have no T cells, B cells, NK cells and ILCs. After engraftment, airway inflammation will be induced by intranasal administration of IL-33, IL-25+NMU or HDM. One day after the last treatment, severity of airway inflammation will be determined. In the absence of lymphocyte CGRP expression, Applicants expect to see highly-pro-inflammatory ILCs that drive airway inflammation. Since neurons are also major producers of CGRP, it is possible Applicants will need to cross RAG2−/− IL-2Rγ−/− mice onto a CGRP−/− background and then transfer WT or CGRP−/− ILCs. Mice will then be treated with IL-33, IL-25+NMU or HDM to induce airway inflammation and disease severity would be assessed as above.

CGRP inhibits IL-5 and IL-13 production in IL-33 activated ILCs, while enhancing Amphiregulin expression in vitro. These data suggest that CGRP may transform pro-inflammatory ILCs into homeostatic, tissue-protective ILCs. To identify surface molecules, soluble factors and signaling pathways that might contribute to the tissue protective state of ILCs, Applicants will culture lung-resident ILCs in vitro with IL-33 or IL-33+CGRP and analyze them after three days by population RNA-seq. Differentially regulated genes between IL-33- and IL-33+CGRP stimulated ILCs will be validated by quantitative Nanostring multiplex expression analysis and flow cytometry. Applicants will determine whether CGRP alters expression of the pro-inflammatory signature genes (Wallrapp, et al.), similar to how it suppresses IL-5 and IL-13. Applicants will particularly focus on whether there is an increase in expression of inhibitory genes, as well as genes such as amphiregulin that promote repair. Applicants have previously shown that proinflammatory ILC2s form a distinct cluster of cells in the in vivo scRNA-seq data set. Thus, Applicants will also undertake scRNA-seq after in vivo treatment with IL-33+CGRP, compared with IL-33 alone. Relative cluster composition will be analyzed to assess if CGRP administration results in the loss of cell clusters that score highly for the proinflammatory gene signature, or if it instead drives acquisition of an inhibitory gene signature in a novel cluster of cells. In this regard, it is intriguing that Applicants observe that two clusters of lung ILCs have significantly lower Ramp1 expression than the others, and one of these clusters has a transcriptional profile similar to that of a previously defined highly inflammatory population (Cluster 8). This suggests that this population may be less susceptible to CGRP-mediated inhibition, thus promoting a pro-inflammatory phenotype. Since Applicants have a transcriptional profile for each cluster, Applicants can determine whether CGRP administration in vivo also induces a cluster of cells with a regulatory phenotype. If this is the case, the scRNA-seq analysis will provide us with a unique way to identify the cell populations/phenotypes induced by CGRP that might regulate the development of allergic inflammation.

By creating a detailed transcriptional atlas of ILCs in both health and after allergen challenge, Applicants expect to identify a variety of novel genes involved in regulating allergic responses. Additionally, analysis of ILC transcriptional profiles after allergen challenge will be facilitated by the prior studies of ILC profiles after alarmin stimulation. Applicants can identify and validate a number of novel therapeutic targets, with a goal of ultimately ameliorating the toll of asthma and other atopic diseases by perturbing specific neuro-immune axis operational in affected individuals.

The scRNA-seq analysis has identified 129 genes whose expression is up- or down-regulated when ILC2s are co-activated by IL-25 together with NMU. These genes therefore distinguish pro-inflammatory ILC2s from homeostatic ILC2s. Moreover, many of these genes are not specific for IL-25+NMU activation, but show similar expression patterns in pro-inflammatory ILC2s generated following activation with IL-33 or allergic challenge with HDM, suggesting that they may function as novel regulators of pro-inflammatory ILC2 function (FIG. 8). Therefore, these potential novel regulators of the pro-inflammatory ILC2 signature may affect ILC2 function and regulate development of allergic airway inflammation.

Four potential novel regulators with distinct patterns of expression in pro-inflammatory ILC2s, Nr4a1, Ctla4, Il1r2 and Tnfrsf8 (CD30), have not been studied previously. Applicants hypothesized that upregulation of IL1R2 and CD30 may promote generation of pro-inflammatory ILC2s, while upregulation of CTLA4 and Nr4a1 may inhibit ILC2 expansion and cytokine production, therefore limiting the development of allergic inflammation. The role of CTLA4 may be similar to that of the immune checkpoint molecule PD-1, which has been shown to inhibit ILC expansion and effector function.

CTLA4−/−, CD30−/−, IL1R2−/−, and Nr4a1−/− mice are available. Applicants will isolate ILCs (defined as IL-7Rα+ CD90+ Lin-cells), by FACS-sorting from the lungs of Nr4a1−/−, CD30−/−, IL1R2−/−, or CTLA4−/− mice, and wild type controls. ILCs will be stimulated in vitro with IL-7 alone, or in conjunction with IL-33, IL-25, and IL-25+NMU. After three days in culture, supernatants will be collected and analyzed for cytokine expression using the LegendPlex mouse T helper panel (allowing simultaneous analysis of IL-2, 4, 5, 6, 9, 10, 13, 17A, 17F, 21, 22, TNFα, and IFNγ) and ELISA (for Amphiregulin). RNA will also be isolated from the cells at this time point, and analyzed either by qPCR for expression of Il5, Il13, and Areg, or by Nanostring, using a custom codeset containing probes for the 129 genes differentially expressed in IL-25+NMU-treated ILCs, along with other immunologically relevant genes. To assess proliferation, Applicants will both label ILCs with CellTrace Violet for flow cytometric analysis, as well as pulse cells with 3H-thymidine and measure incorporation. Applicants anticipate that Applicants will observe enhanced proliferation and cytokine expression by CTLA4−/− and Nr4a1−/− ILCs, while CD30−/− and IL1R2−/− ILCs will have decreased responses to alarmin activation. However, by using multiplex assays such as Legendplex and Nanostring, Applicants will be able to assess unanticipated phenotypes patterns of cytokine or gene expression.

Applicants will assess the role of Nr4a1, CD30, IL1R2, and CTLA4 on ILC responses in vivo. Applicants will intranasally administer either IL-25+NMU daily for three consecutive days or HDM on days 0, 7, 8, and 9 to Nr4a1−/−, CD30−/−, IL1R2−/−, or CTLA4−/− mice, or wild type controls. BAL and lung homogenates will be collected from all mice, and analyzed by flow cytometry for key cell populations (including eosinophils, neutrophils, ILCs, and Th2 cells). Additionally, cytokine expression in BALF will be assessed by LegendPlex, and expression of relevant cytokines (e.g. Il5, Il13, Areg) in lung homogenates will be analyzed by qPCR. In some experiments, mice will undergo graded methacholine challenge and airway resistance will be measured to assess for airway hyper-reactivity. Finally, the post-caval lobe of the right lung will be fixed for histologic analysis to determine infiltration of pro-inflammatory cells in the lung (H&E staining) and goblet cell hyperplasia (PAS staining).

For those genes with promising phenotypes either in vitro or in vivo, Applicants will then directly analyze the global transcriptional profiles of ILCs using scRNA-seq following challenge with either IL-25+NMU or HDM. Applicants will sort ILCs from the lungs of Nr4a1−/−, CD30−/−, IL1R2−/−, or CTLA4−/− mice, or wild type controls, and generate scRNA-seq libraries using 10× droplet-based technology. The lung ILC transcriptional atlas described herein has distinct clusters of cells with inflammatory and proliferative phenotypes, and Applicants will be able to assess both if loss of these putative novel regulators alters the relative size of these clusters, as well as for differences in pro-inflammatory and regulatory gene expression.

### Methods

Mice and in vivo ILC activation. C57B1/6J mice were purchased from the Jackson Laboratory. Nmur1-LacZ reporter mice with a LacZ cassette knocked into the Nmur1 locus were rederived from Nmur1^{tm1.1(KOMP)Vlcg} sperm obtained from the trans-NIH Knock-Out Mouse Project (KOMP) Repository. NMU-deficient mice (NMU-KO) were rederived from B6.129S2-Nmu<tm1Mko> embryos from the RIKEN BioResource Center. For experiments with Nmur1-LacZ (Nmur1-KO) and NMU-KO mice, littermates that were either homozygous or heterozygous for the wild type allele were used as controls. Mice were housed under specific-pathogen-free conditions. For experiments, mice were matched for sex and age, and most mice were 6-10 weeks old. Where indicated, mice were anesthetized with Isoflurane and treated intranasally with the indicated stimuli (500 ng IL-25, 500 ng IL-33, or 20 μg Neuromedin U) daily for three consecutive days. The total administered volume was 20 μl for all conditions. Mice were randomly assigned to treatment groups after matching for sex and age. Airway inflammation was also induced with house dust mite (HDM) extract (Greer Laboratories). Mice were treated intranasally with 10 μg HDM on day 0, 7, 8, and 9, prior to sacrifice on day 10. All experiments were conducted in accordance with animal protocols approved by the Harvard Medical Area Standing Committee on Animals or BWH IACUC.

Flow cytometry. For flow cytometric analysis CD38 (clone: 145-2C11), CD4 (clone: RM4-5), CD8a (clone: 53-6.7), CD11b (clone: M1/70), CD11c (clone: N418), CD19 (clone: 6D5), CD30 (Tnfrsf8; clone: mCD30.1), CD45 (clone: 30-F11), CD47 (clone: miap301), CD48 (clone: HM48-1), CD81 (clone: Eat-2), CD90.2 (clone: 30-H12), CD127 (clone: A7R34), CD152 (CTLA-4; clone: UC10-4B9), I-A/I-E (clone: M5/114.15.2), IL-5 (clone: TRFK5), KLRG1 (clone: 2F1/KLRG1), NK1.1 (clone: PK136), Sema4A (clone: 5E3/SEMA4A), ST2 (clone: DIH9), TCRβ (clone: H57-597) and TCRγδ (clone: GL3) were purchased from BioLegend. 7AAD was obtained from BD Pharmingen, CD121b (IL1r2; clone: 4E2), Batf (clone: S39-1060) and Siglec-F (clone: E50-2440) from BD Biosciences and CD85k (gp49; clone: H1.1), Fixable Viability Dye eFluor 506, Galectin-3 (Lgals3; clone: eBioM3/38), IL-13 (clone: eBiol3A), IL17RB (IL-25R; clone: Munc33), Ki-67 (clone: SolA15) and Nur77 (Nr4a1) (clone: 12.14) from eBioscience. Cells were stained on ice with antibodies for surface molecules and the live/dead marker 7AAD and analyzed on a LSRFortessa (BD Biosciences). Intracellular cytokine staining was performed after incubation for 5 hr with 1 uM ionomycin (Sigma-Aldrich), 50 ng/ml phorbol 12-myristate 13-acetate (Sigma-Aldrich) and GolgiStop (BD Biosciences). Cells were then fixed and stained using the BD Cytofix/Cytoperm buffer set (BD Biosciences) per manufacturer's instructions. Proliferation was assessed by Ki-67 staining after cell fixation and permeabilization using the Foxp3/Transcription Factor Staining Buffer Set (eBioscience). Different cell types were identified by the following gating strategies: ST2⁺ ILCs (7AAD⁻ CD45⁺ CD4⁻ Lineage⁻ CD90.2⁺ CD127⁺ ST2⁺), T cells (7AAD⁻ CD45⁺ CD4⁺), B cells (7AAD⁻ CD45⁺ CD19+), eosinophils (7AAD⁻ CD45⁺ CD11b⁺ CD11c^{low} Siglec-F⁺ SSC^{high}), neutrophils (7AAD⁻ CD45⁺ CD11c^{low} CD11b⁺ Ly6G⁺ CD11b⁺), alveolar macrophages (7AAD⁻ CD45⁺ CD11c^{high} CD11b^{intermediate}) and CD45⁻ cells (7AAD⁻ CD45⁻).

Lung analysis. Mice were sacrificed and perfused with cold PBS. Where indicated, after perfusion, broncho-alveolar lavage (BAL) was obtained by injecting 1.5 ml cold PBS into the lungs via a secured tracheal cannula. BALF was centrifuged, and the supernatant was used for analyzing cytokine levels and the cell pellet was resuspended, counted, and used for flow cytometry. Following BAL, lung lobes were dissected. The post-caval lobe was fixed in buffered formalin for histological analysis. Single cell suspensions of the remaining lung parenchymal tissue were prepared with the GentleMACS lung dissociation kit (Miltenyi Biotec) according to the manufacturer's instructions. Where indicated, cells were diluted in 10% Trypan Blue and viable cells counted using a hemocytometer.

Fluorescence-activated cell sorting of innate lymphoid cells. After dissociation, single cell suspensions were incubated with CD90.2 MicroBeads (Miltenyi Biotec) on ice and enriched for CD90.2⁺ cells by magnetic separation using LS columns according to the manufacturer's protocol. CD90.2⁺ lung cells were then stained on ice with antibodies for sorting. ILCs were defined as 7AAD⁻ CD45⁺ CD90.2⁺ CD127⁺ Lineage (CD11b, CD11c, CD19, NK1.1, CD36, CD4, CD8a, TCRβ, TCRγδ)⁻ cells and sorted on a BD FACS Aria (BD Biosciences).

RNA-Seq. For population (bulk) RNA-seq, sorted ILCs were lysed with RLT Plus buffer and RNA was extracted using the RNeasy Plus Mini Kit (Qiagen). Full-length RNA-seq libraries were prepared as previously described (Singer, M. et al. A Distinct Gene Module for Dysfunction Uncoupled from Activation in Tumor-Infiltrating T Cells. Cell 166, 1500-1511 e1509, doi:10.1016/j.cell.2016.08.052 (2016). and paired-end sequenced (75 bp×2) with a 150 cycle Nextseq 500 high output V2 kit.

For droplet-based 3′ end massively parallel single-cell RNA sequencing (scRNA-seq), sorted ILCs were encapsulated into droplets, and libraries were prepared using Chromium™ Single Cell 3′ Reagent Kits v2 according to manufacturer's protocol (10× Genomics). The generated scRNA-seq libraries were sequenced using a 75 cycle Nextseq 500 high output V2 kit.

For full-length scRNA-Seq, single ILCs were sorted into 96-well plates containing 5 ul TCL Buffer (QIAGEN) with 1% 2-Mercaptoethanol, centrifuged and frozen at −80° C. SMART-Seq2 protocol was carried out as previously described²³ with minor modifications in the reverse transcription step. cDNA was amplified with 22 cycles and fragmented with one-eighth of the standard Illumina NexteraXT reaction volume. Single-cell libraries were pooled and paired-end sequenced (38 bp×2) with a 75 cycle Nextseq 500 high output V2 kit.

All RNA-Seq data represent pooled data from at least two distinct biological replicates.

ILC in vitro culture. For in vitro experiments 5,000 ILCs/well were cultured in a 96 well round bottom plate with 20 ng/ml IL-7 (R&D Systems), 200 ng/ml IL-25 (R&D Systems) or 20 ng/ml, 2 ng/ml or 0.2 ng/ml IL-33 (BioLegend) with or without 1 μg/ml Neuromedin U (US Biological). In some cases purified CD90.2⁺ lung cells were first labeled with CellTrace Violet (Thermo Fisher Scientific), then sorted as described above, and cultured for 3 days under the indicated conditions.

Histology. Following paraffin embedding, sections of the formalin-fixed lung lobe were stained by H&E staining. Tissue sections were scored by a histopathologist in a blinded manner for severity of lung inflammation according to the following scoring system: 0=normal, 1=very mild, 2=mild, 3=moderate or 4=severe.

Methacholine challenge. Airway hyperresponsiveness was determined as previously described (Talbot, S. et al. Silencing Nociceptor Neurons Reduces Allergic Airway Inflammation. Neuron 87, 341-354, doi:10.1016/j.neuron.2015.06.007 (2015)) using a flexiVent rodent ventilator (SciReq).

LacZ reporter assay. The Nmur1 null allele contains a LacZ reporter cassette. Single cell suspensions of lung cells from Nmur1-LacZ^{+/-} mice were stained with the FluoReporter lacZ flow cytometry kit (Thermo Fisher Scientific) according to the manufacturer's protocol. Immediately after fluorescein di-V-galactoside (FDG) loading was stopped with 1.8 ml ice-cold medium, cells were stained with 7AAD and antibodies against surface markers and analyzed by flow cytometry.

Quantitative real-time PCR. RNA was isolated using RNeasy Plus Mini Kit (Qiagen) and reverse transcribed to cDNA with iScript cDNA Synthesis Kit (Bio-Rad). Gene expression was analyzed by quantitative real-time PCR on a ViiA7 System (Thermo Fisher Scientific) using TaqMan Fast Advanced Master Mix (Thermo Fisher Scientific) with the following primer/probe sets: Il5 (Mm00439646_m1), Il13 (Mm00434204_m1), Il17rb (Mm00444709_m1), Nmur1 (Mm04207994_m1), Nmur2 (Mm00600704_m1), Nmu (Mm00479868_m1) and Actb (Applied Biosystems). Expression values were calculated relative to Actb detected in the same sample by duplex qPCR.

Cytokine quantification. Cytokine concentrations in BAL fluid, lung and supernatant of in vitro ILC cultures were analyzed by the LegendPlex Mouse Th Cytokine Panel (13-plex) (BioLegend) according to the manufacturer's instructions and analyzed on a FACS Calibur (BD Biosciences).

T cell in vitro culture. CD4⁺ T cells were isolated as described previously⁴⁶ and sorted for naive T cells (CD4⁺ CD62L⁺ CD44^{low}) on a FACS Aria. Naive T cells were cultured in the presence of plate-bound anti-CD3 (1 μg/ml; Bio X Cell) and anti-CD28 (1 μg/ml; Bio X Cell) antibodies. Th2 cells were generated by addition of 20 ng/ml IL-4 (Miltenyi Biotec) and 20 ng/ml anti-IFNγ (Bio X Cell) antibody. On day 3 of in vitro differentiation, PBS, 200 ng/ml IL-33 (BioLegend) or 100 ng/ml IL-25 (R&D Systems) were added to the T cell culture either with or without 1 μg/ml NMU (US Biological). After 2 additional days, RNA was isolated.

Nodose/jugular and dorsal root ganglion isolation and cultures. Nodose/jugular ganglion and dorsal root ganglia (DRG) were dissected from mice and dissociated in 1 mg/mL Collagenase A with 3 mg/ml dispase II (Roche Applied Sciences) in HEPES buffered saline (Sigma) for 60 minutes at 37° C. For some experiments, cells were then lysed in RLT Plus buffer and RNA was isolated using the RNeasy Plus Mini Kit (Qiagen). For the purposes of cell culture, the DRG cell suspension was then triturated with glass pasteur pipettes of decreasing size, followed by centrifugation over a 12% BSA (Sigma) gradient. After centrifugation, the top layer of neuronal debris was discarded and the DRG pellet was resuspended in neurobasal (NB) media containing B-27 and penicillin/streptomycin (Life Technologies). DRGs were then plated on laminin-coated 96-well culture dishes in NB media with B27, 50 ng/ml nerve growth factor (NGF) and penicillin/streptomycin. The next day the cells were washed with PBS prior to addition of fresh NB media containing B-27, NGF and penicillin/streptomycin. DRG cultures were stimulated with 200 ng/ml IL-13 for 30 minutes, at which time RNA was isolated for qPCR analyses.

Immunofluorescence Microscopy. Mice were perfused with 37° C. PBS via the heart. The lungs were extracted and inflated via the trachea with 4% low melting agarose (16520-100; Invitrogen) and fixed in 4% PFA on ice for 1 hour. The lungs were embedded in agarose for vibratome cutting (Leica). 100 μm lung slices were blocked first with the mouse on mouse blocking reagent (Vector Laboratories) and subsequently with 5% goat and donkey serum (Jackson ImmunoResearch) in PBS/0.1% Triton-X-100. Tissue was stained for rat anti-CD3F (17A2; BioLegend), hamster anti-KLRG1 (2F1; eBioscience) and mouse anti-SNAP25 (SMI81; BioLegend) overnight at 4° C. shaking. After washing in PBS, tissues were incubated at room temperature for 1 h in in PBS/0.1% Triton-X-100 containing goat anti-rat-AF555, goat anti-hamster-AF647, or goat anti-mouse IgG1-AF488 (all ThermoFisher Scientific) and then washed again. Images were acquired with an inverted Nikon Eclipse Ti microscope (Nikon). Z-stacks were acquired and converted into all-in-focus images using the Extended Depth of Focus (EDF) plug-in (NIS-Elements). Distances of KLRG1⁺ CD3ε⁻ cells to the closest SNAP-25⁺ nerve fiber were measured using the NIS-Elements software.

Statistical analysis of functional data. No data were excluded from analysis. Prism 7 (GraphPad Software) was used to perform two-tailed t-test and ordinary one-way or two-way ANOVA with Tukey's multiple comparisons test on datasets for which statistical significance is indicated (except the RNA-sequencing data). All figures of functional data show mean±SEM.

P values in transcriptomic analysis. For certain types of numeric computations, the smallest P value that R can report is “<2.2×10⁻¹⁶”.

Analysis of droplet-based scRNA-Seq data: Initial QC. Gene counts were obtained by aligning reads to the mm10 genome using CellRanger software (v1.2 for data from alarmin-treated and NMU-treated mice, v1.3 for data from HDM-treated mice) (10× Genomics), with the genome reannotated at the 3′ end of Nmur1. To remove doublets and poor-quality cells, cells were excluded from subsequent analysis if they were outliers in their sample of origin in terms of number of genes, number of UMIs, and percentage of mitochondrial genes. The number of UMIs per cell and number of genes expressed per cell are tightly correlated with condition, likely due to the effect of proliferation on transcript numbers. Sample-specific cutoffs ranged from 626-2,483 genes per cell for a PBS treated sample to 1,502-5,260 genes per cell for an IL-33 treated sample. At least 92% of cells were retained for each sample.

To further estimate and remove technical variability from the overall increased variability across replicates in Group C (defined below), additional QC measures were taken. UMI and gene saturation were estimated independently for each cell by subsampling a fraction of the total number of reads, with replacement, across a range of fractions (0.02 to 0.98, in 0.02 increments). For each subsample, Applicants calculated the number of UMIs and transcripts detected. The sampling procedure was repeated 10 times, and the values were used to estimate saturation limits for UMI/genes by nonlinear fitting of the following saturation function: y=ax/(b+x)+c. Cells were removed if they were outliers with respect to estimated saturation for either genes or UMIs. Cells were also removed if they were outliers in terms of the ratio or relative difference of the total number of UMIs with the number of unique UMIs. After all QC, 73-83% of cells in each of these samples were retained.

Parts of the subsequent analysis utilized the R package Seurat⁴⁷, version 1.4.0.7, which includes sparse matrix support for large datasets. To normalize gene counts while accounting for widely varying UMI counts among conditions, Applicants used a scaling factor reflecting the expected number of UMIs in each condition. Let w_{c,i} be the mean number of UMIs per cell in condition c, batch i. Seurat's LogNormalize( ) function was called on cells from condition c with the scale factor argument set to:

10,000×(w_{c,i}/meanᵢ(w_{control,i}))

Applicants refer to the output values as logTPX (as opposed to the default logTPM).

The 63,152 high-quality cell profiles were combined into three (non-exclusive) groups.

Group A (24,187 cells): cells stimulated with PBS (9,623 cells), IL-25 (6,849 cells), or IL-33 (7,715 cells).

Group B (35,542 cells): cells stimulated with PBS (9,623 cells), NMU (9,698 cells), IL-25 (6,849 cells), or IL-25+NMU (9,372 cells).

Group C (21,895 cells: cells from WT mice stimulated with PBS (5,393 cells) or HDM (6,280 cells), as well as Nmur1-KO mice stimulated with PBS (4,191 cells) or HDM (6,031 cells).

To verify that the dataset consists of ILCs, Applicants checked the raw counts for the expression of major markers of other immune cell groups. For subsequent analysis, genes expressed in less than 0.1% of cells were excluded.

Analysis of droplet-based scRNA-Seq samples: Signature scores. Applicants calculated signature scores as the log of the geometric mean of the TPX values for the genes in the signature. That is, let S be a set of m genes defining a signature, and for any gene g in S and a given cell, let x_{g} be the expression of g in the cell in TPX. Then the signature score for that cell is calculated as

log(II_{g}(x_{g}+1)^{1/m})

which is equivalent to the arithmetic mean of the logTPX values. The geometric mean lessens the impact of any specific gene's range of expression on the score. Actual expression values, rather than centered or z-scored expression values were used. For several of the signatures, centering (or z-scoring) expression values before computing signatures leads to misleading scores that are close to 0 across the whole dataset, though the corresponding gene expression is high. This is in large part due to ILC2s composing the majority of the cells that Applicants analyzed, and hence genes that are highly expressed by ILC2s lack sufficient variance over the data set to be useful in a mean-centered signature.

Applicants also did not replace these scores with a statistical comparison of them to randomized signatures selected from a null distribution (in contrast to Applicants other studies; Tirosh, I. et al. Dissecting the multicellular ecosystem of metastatic melanoma by single-cell RNA-seq. Science 352, 189-196; and Singer, M. et al. A Distinct Gene Module for Dysfunction Uncoupled from Activation in Tumor-Infiltrating T Cells. Cell 166, 1500-1511 e1509). Due to the varying proliferative responses in the cells, it is difficult to find a true null set of signatures, even after matching genes for dataset-wide mean and variance profiles. Signature scores are thus calculated in a way that is independent of the expression levels of unrelated genes in the same cell, and may be interpreted as similar to, though less noisy than, single-gene expression values.

The ILC subset signatures (ILC1, 2, 3) were curated based on established markers for ILC subsets (Table 1). The proliferation signature was created by combining the previously published gene signatures (Kowalczyk, M. S. et al. Single-cell RNA-seq reveals changes in cell cycle and differentiation programs upon aging of hematopoietic stem cells. Genome Res 25, 1860-1872, doi:10.1101/gr.192237.115 (2015); and Tirosh, I. et al. Dissecting the multicellular ecosystem of metastatic melanoma by single-cell RNA-seq. Science 352, 189-196, doi:10.1126/science.aad0501 (2016)) that define G1-S and G2-M phases (Table 2). For both ILC subset and proliferative signatures, all genes contribute positively to the signature score. For the inflammatory ILC2 signature, genes contribute negatively to the score if they are down-regulated in NMU+IL-25 relative to IL-25 (and positively otherwise) (Table 5).

TABLE 1

| Signature | Sign | Gene |
|---|---|---|
| ILC1 | plus | Tbx21 |
| ILC1 | plus | Ifng |
| ILC1 | plus | Il21r |
| ILC1 | plus | Ccl5 |
| ILC1 | plus | Ccl4 |
| ILC1 | plus | Ccl3 |
| ILC1 | plus | Ncr1 |
| ILC1 | plus | Il15r |
| ILC1 | plus | Eomes |
| ILC1 | plus | Cxcr3 |
| ILC1 | plus | Il12rb1 |
| ILC2 | plus | Gata3 |
| ILC2 | plus | Lmo4 |
| ILC2 | plus | Areg |
| ILC2 | plus | Ccl1 |
| ILC2 | plus | Csf2 |
| ILC2 | plus | Il4 |
| ILC2 | plus | Il5 |
| ILC2 | plus | Il13 |
| ILC2 | plus | Cxcl2 |
| ILC2 | plus | Il9 |
| ILC2 | plus | Il1rl1 |
| ILC2 | plus | Il9r |
| ILC2 | plus | Il17rb |
| ILC2 | plus | Klrg1 |
| ILC3 | plus | Rorc |
| ILC3 | plus | Tcf7 |
| ILC3 | plus | Batf3 |
| ILC3 | plus | Il17f |
| ILC3 | plus | Il17a |
| ILC3 | plus | Il22 |
| ILC3 | plus | Ncr1 |
| ILC3 | plus | Il1r1 |
| ILC3 | plus | Ahr |
| ILC3 | plus | Il23r |
| ILC3 | plus | Cxcr5 |
| ILC3 | plus | Ccr6 |

TABLE 2

| Signature | Sign | Gene |
|---|---|---|
| Proliferation | plus | Mcm5 |
| Proliferation | plus | Pcna |
| Proliferation | plus | Tyms |
| Proliferation | plus | Fen1 |
| Proliferation | plus | Mcm2 |
| Proliferation | plus | Mcm4 |
| Proliferation | plus | Rrm1 |
| Proliferation | plus | Ung |
| Proliferation | plus | Gins2 |
| Proliferation | plus | Mcm6 |
| Proliferation | plus | Cdca7 |
| Proliferation | plus | Dtl |
| Proliferation | plus | Prim1 |
| Proliferation | plus | Uhrf1 |
| Proliferation | plus | Mlf1ip |
| Proliferation | plus | Hells |
| Proliferation | plus | Rfc2 |
| Proliferation | plus | Rpa2 |
| Proliferation | plus | Nasp |
| Proliferation | plus | Rad51ap1 |
| Proliferation | plus | Gmnn |
| Proliferation | plus | Wdr76 |
| Proliferation | plus | Slbp |
| Proliferation | plus | Ccne2 |
| Proliferation | plus | Ubr7 |
| Proliferation | plus | Pold3 |
| Proliferation | plus | Msh2 |
| Proliferation | plus | Atad2 |
| Proliferation | plus | Rad51 |
| Proliferation | plus | Rrm2 |
| Proliferation | plus | Cdc45 |
| Proliferation | plus | Cdc6 |
| Proliferation | plus | Exo1 |
| Proliferation | plus | Tipin |
| Proliferation | plus | Dscc1 |
| Proliferation | plus | Blm |
| Proliferation | plus | Casp8ap2 |
| Proliferation | plus | Usp1 |
| Proliferation | plus | Clspn |
| Proliferation | plus | Pola1 |
| Proliferation | plus | Chaf1b |
| Proliferation | plus | Brip1 |
| Proliferation | plus | E2f8 |
| Proliferation | plus | Hmgb2 |
| Proliferation | plus | Cdk1 |
| Proliferation | plus | Nusap1 |
| Proliferation | plus | Ube2c |
| Proliferation | plus | Birc5 |
| Proliferation | plus | Tpx2 |
| Proliferation | plus | Top2a |
| Proliferation | plus | Ndc80 |
| Proliferation | plus | Cks2 |
| Proliferation | plus | Nuf2 |
| Proliferation | plus | Cks1b |
| Proliferation | plus | Mki67 |
| Proliferation | plus | Tmpo |
| Proliferation | plus | Cenpf |
| Proliferation | plus | Tacc3 |
| Proliferation | plus | Fam64a |
| Proliferation | plus | Smc4 |
| Proliferation | plus | Ccnb2 |
| Proliferation | plus | Ckap2l |
| Proliferation | plus | Ckap2 |
| Proliferation | plus | Aurkb |
| Proliferation | plus | Bub1 |
| Proliferation | plus | Kif11 |
| Proliferation | plus | Anp32e |
| Proliferation | plus | Tubb4b |
| Proliferation | plus | Gtse1 |
| Proliferation | plus | Kif20b |
| Proliferation | plus | Hjurp |
| Proliferation | plus | Hjurp |
| Proliferation | plus | Cdca3 |
| Proliferation | plus | Hn1 |
| Proliferation | plus | Cdc20 |
| Proliferation | plus | Ttk |
| Proliferation | plus | Cdc25c |
| Proliferation | plus | Kif2c |
| Proliferation | plus | Rangap1 |
| Proliferation | plus | Ncapd2 |
| Proliferation | plus | Dlgap5 |
| Proliferation | plus | Cdca2 |
| Proliferation | plus | Cdca8 |
| Proliferation | plus | Ect2 |
| Proliferation | plus | Kif23 |
| Proliferation | plus | Hmmr |
| Proliferation | plus | Aurka |
| Proliferation | plus | Psrc1 |
| Proliferation | plus | Anln |
| Proliferation | plus | Lbr |
| Proliferation | plus | Ckap5 |
| Proliferation | plus | Cenpe |
| Proliferation | plus | Ctcf |
| Proliferation | plus | Nek2 |
| Proliferation | plus | G2e3 |
| Proliferation | plus | Gas2l3 |
| Proliferation | plus | Cbx5 |

Analysis of droplet-based scRNA-Seq samples: Assigning ILC type. ILC signatures were used to assign each cell to one of the following categories: ILC1, ILC2, ILC3, “mixed” (scoring highly for multiple ILC types), and “none” (not scoring highly for any ILC type). The frequency of mixed type ILCs (2.6%) is comparable to the expected doublet rate (3-4%). Based upon the dip in the bimodal distributions of ILC subset signature scores, the minimum score for assignment to a given category was set to 0.08. To be uniquely assigned to a category, the ratio of the highest score to the next highest score was required to be at least 1.25. The analysis is not sensitive to the specific ratio threshold choice of 1.25; that selection was made to balance the trade-off between the purity of the transcriptional profile of cells assigned to one of the three ILC subtype populations, and the number of cells called as mixed.

To test the strength of association between ILC type and treatment conditions, Applicants used the R package nnet, version 7.3-12, to do a multinomial logistic regression on the ILC type, with replicate and condition as predictors.

Analysis of droplet-based scRNA-Seq samples: PCA, clustering, and tSNE. Variable genes were then selected using the MeanVarPlot function in Seurat with the x.low.cutoff and y.cutoff parameters set to 0.05 and 0.7, respectively, resulting in gene sets of size 774 (Group A), 723 (Group B), and 475 (Group C). PCAFast was run on mean-centered variable genes to compute a limited number of PCs. To select the number of PCs to include for subsequent analysis, Applicants estimated the number of eigenvalues larger than would be predicted by a null distribution for random matrices (Marchenko-Pastur law), and also assessed the decrease in marginal proportion of variance explained with larger PCs. The top 22 (Groups A, B) and 13 (Group C) PCs were included for subsequent analysis. Applicants confirmed that the resulting analyses were not particularly sensitive to this exact choice.

The cells were clustered via Seurat's FindClusters function, which optimizes a modularity function on a k-nearest-neighbor graph computed from the top eigenvectors. After a range of cluster resolution parameters were tested, 0.6 (Groups A and B) and 0.5 (Group C) were selected because resulting clusters captured major, condition-related divisions, known subgroups, and statistically validated transcriptional distinctions of interest, while avoiding subdivisions of relatively uniform parts of the data.

To visualize the data, tSNE plots were created by calling Seurat's RunTSNE function, with the dims.use parameter set to the selected number of significant PCs and the do.fast parameter set to TRUE. A number of perplexity parameter choices were evaluated before selecting 100 (Group A) and 50 (Groups B and C). These perplexity settings produced tSNE plots that reflected the cluster structure found independently of tSNE, without introducing extreme artifacts. TSNE plots of cells separated by batch indicate that experimental batches appear to have a relatively minor impact on the PCA and clustering.

Analysis of droplet-based scRNA-Seq samples: Differential gene expression. To avoid spurious results due to cells in different conditions or clusters having vastly different amounts of mRNA, differential expression (DE) analysis accounted for the varying number of transcripts and genes in each condition. Applicants fit raw counts to a mixture of generalized linear models that include covariates for the log of the number of UMIs in a cell, as well as a factor for the batch. Specifically, Applicants fit a zero-inflated negative binomial model using the zeroinfl function⁴⁸ from the pscl R package⁴⁹, version 1.4.9. The zero-inflated negative binomial model combines a count component and a point mass at zero, which is relevant for scRNA-Seq data where zero values are significantly inflated due to the technology not capturing expressed genes, particularly those with low expression¹¹. The model requires a substantial amount of data to fit, making it well suited to data generated by massively-parallel methods. As an alternative to the zero-inflated negative binomial, Applicants also performed a logistic regression by fitting a generalized linear model using the binomial family with a logit link, with the same covariates.

DE tests included the following models: (1) a cluster-based model with indicator coefficients for each cluster except the reference PBS-dominated cluster (Groups A, B, and C); (2) a condition-wide model with indicator coefficients for each condition (with the control condition as reference) (Groups A, B, and C); and (3) a direct comparison of IL-25 vs. IL-33, with IL-33 as reference (Group A); (4) a condition-based model with indicator coefficients for NMU and IL-25, and an additional interaction term (with the control condition as reference) to detect non-additive effects (Group B); (5) a direct comparison between IL-25+NMU and IL-25, with IL-25 as reference (Group B). In Group C, Applicants restricted the DE analysis to the cells transcriptionally classified as ILC2s, in order to identify difference in these particular cells, without the analysis being driven by the change in relative proportions of ILC1s and ILC3s compared to ILC2s after HDM treatment.

Many cell-cycle genes and ribosomal protein genes are differentially expressed across conditions and clusters, particularly if there is a difference in proliferation. To detect other differentially expressed genes as well, before ranking DE results, Applicants removed ribosomal protein genes and (in all cases except Group C, where it was not needed), the genes in the proliferation signature.

DE tests report coefficients and associated p-values for each variable of interest (e.g., cluster or condition), separately for each model component. To rank the results for any given model, Applicants created a list of differentially expressed candidate genes that are detected in at least 10% (15% for Group C) of the cells in one of the groups in the model and have a coefficient for a term of interest with absolute value at least 0.5 (0.75 for Group C) and corresponding FDR-adjusted p-value<1×10⁻²⁰ for condition-wide DE, <1×10⁻⁶ for cluster DE, for at least one component. Applicants ranked these candidates by lowest p-value and also by largest absolute value of coefficient. The top 25 (5 and 3 for cluster models in Groups A-B and C, respectively) genes according to each ranking were reported, with a minimum of 1-2 genes, if available, selected from each of the set of candidates with positive coefficients and the set with negative coefficients. These genes are reported according to the condition or cluster for which they were ranked highly, and the sign reported is “plus” (“minus”) if that condition or cluster has a higher (lower) expression by both fraction of cells expressing and level of expression than the reference, or if these are discordant then the sign is reported as “NA”.

Applicants curated a representative selection from the highest-ranked results to represent, common, distinctive patterns across clusters in Group A, and patterns that distinguish IL-25+NMU from the other conditions in Group B and highlight non-linear interactions between NMU and IL-25. To create the inflammatory ILC2 signature. Applicants used the top-ranked genes differentially expressed between HDM and PBS treatments in ILC2s only in WT mice, as well as those differentially expressed in HDM- and ILC2-dominated clusters (5 and 6) in this dataset. The signature genes are reported with the sign positive if the respective condition or cluster has higher expression than the reference for that model.

To more broadly compare differentially expressed genes between HDM and IL-25+NMU datasets, Applicants constructed one gene set for IL-25+NMU by taking the top differentially expressed genes for all models and comparisons in Group B in which there is a coefficient for the condition IL-25+NMU or a coefficient for a group or cluster to which IL-25+NMU-treated cells contribute significantly (clusters 2, 6-11). Applicants then performed the analogous procedure in the HDM data (including clusters 1-2 and 4-7). Using as the null set the 12,719 genes shared between Groups B and C, Applicants used Fisher's exact test to determine the significance of the overlap of 23 genes between the 156 genes in the IL-25+NMU gene set (151 of which are in the null set) and 85 from the HDM data (all of which are in the null set) (P<1.64×10⁻²⁵).

Applicants curated a selection from the highest-ranked results to represent distinctive patterns across clusters that distinguish NMU+IL-25 from the other conditions and shed light on the non-linear interactions between NMU and IL-25 in the pro-inflammatory ILC2 signature (Table 5).

TABLE 5

| Signature | Sign | Gene | clu_8_9.v.clu_6 | NMU_IL25.v.IL25 | wint_cond_all.bin | wint_cond_all | cond_all | cond_all.bin |
|---|---|---|---|---|---|---|---|---|
| Inflammatory_ILC2 | plus | AA467197 | TRUE | TRUE | NA | NA | TRUE | TRUE |
| Inflammatory_ILC2 | plus | Anxa2 | TRUE | TRUE | FALSE | TRUE | TRUE | TRUE |
| Inflammatory_ILC2 | plus | Batf | NA | NA | NA | NA | TRUE | TRUE |
| Inflammatory_ILC2 | plus | Ccr7 | TRUE | TRUE | TRUE | NA | NA | NA |
| Inflammatory_ILC2 | plus | Cd47 | NA | TRUE | TRUE | FALSE | FALSE | FALSE |
| Inflammatory_ILC2 | plus | Ctla4 | TRUE | TRUE | NA | NA | TRUE | TRUE |
| Inflammatory_ILC2 | plus | Ets1 | NA | NA | FALSE | TRUE | NA | FALSE |
| Inflammatory_ILC2 | plus | Fas | NA | NA | FALSE | TRUE | NA | FALSE |
| Inflammatory_ILC2 | plus | Gsto1 | NA | FALSE | TRUE | TRUE | FALSE | FALSE |
| Inflammatory_ILC2 | plus | H2-T23 | NA | NA | TRUE | TRUE | NA | NA |
| Inflammatory_ILC2 | plus | Il1r2 | NA | TRUE | NA | NA | NA | TRUE |
| Inflammatory_ILC2 | plus | Il5 | NA | NA | FALSE | TRUE | TRUE | TRUE |
| Inflammatory_ILC2 | plus | Il6 | NA | NA | NA | NA | NA | TRUE |
| Inflammatory_ILC2 | plus | Irf4 | NA | NA | TRUE | NA | NA | TRUE |
| Inflammatory_ILC2 | plus | Lgals3 | TRUE | TRUE | FALSE | TRUE | TRUE | TRUE |
| Inflammatory_ILC2 | plus | Lgmn | NA | NA | NA | NA | TRUE | TRUE |
| Inflammatory_ILC2 | plus | Lilrb4a | NA | NA | TRUE | TRUE | NA | FALSE |
| Inflammatory_ILC2 | plus | Mt1 | NA | TRUE | NA | NA | NA | TRUE |
| Inflammatory_ILC2 | plus | Prdx4 | FALSE | FALSE | TRUE | NA | FALSE | FALSE |
| Inflammatory_ILC2 | plus | Ramp1 | NA | NA | TRUE | TRUE | NA | FALSE |
| Inflammatory_ILC2 | plus | S100a6 | NA | NA | TRUE | TRUE | FALSE | FALSE |
| Inflammatory_ILC2 | plus | Tff1 | TRUE | TRUE | NA | NA | NA | NA |
| Inflammatory_ILC2 | plus | Tnfrsf4 | NA | NA | NA | NA | NA | TRUE |
| Inflammatory_ILC2 | plus | Tnfrsf8 | TRUE | TRUE | NA | NA | NA | NA |
| Inflammatory_ILC2 | minus | Areg | TRUE | NA | TRUE | TRUE | NA | FALSE |
| Inflammatory_ILC2 | minus | Btg1 | NA | NA | TRUE | TRUE | NA | FALSE |
| Inflammatory_ILC2 | minus | Calca | TRUE | NA | NA | TRUE | NA | FALSE |
| Inflammatory_ILC2 | minus | Ccl5 | TRUE | NA | TRUE | FALSE | FALSE | NA |
| Inflammatory_ILC2 | minus | Ccr2 | NA | NA | TRUE | TRUE | FALSE | TRUE |
| Inflammatory_ILC2 | minus | Csf2 | TRUE | TRUE | TRUE | TRUE | TRUE | TRUE |
| Inflammatory_ILC2 | minus | Dgat2 | TRUE | TRUE | TRUE | TRUE | TRUE | FALSE |
| Inflammatory_ILC2 | minus | Fosb | TRUE | TRUE | TRUE | FALSE | TRUE | TRUE |
| Inflammatory_ILC2 | minus | Klf3 | NA | NA | TRUE | NA | FALSE | FALSE |
| Inflammatory_ILC2 | minus | Klf4 | NA | TRUE | FALSE | TRUE | TRUE | FALSE |
| Inflammatory_ILC2 | minus | Lpcat2 | NA | TRUE | TRUE | NA | TRUE | TRUE |
| Inflammatory_ILC2 | minus | Ltb | NA | NA | TRUE | TRUE | NA | FALSE |
| Inflammatory_ILC2 | minus | Nr4a1 | TRUE | NA | TRUE | TRUE | NA | FALSE |
| Inflammatory_ILC2 | minus | Sdc4 | TRUE | TRUE | TRUE | TRUE | NA | FALSE |
| Inflammatory_ILC2 | minus | Stab2 | FALSE | TRUE | TRUE | NA | NA | FALSE |
| Inflammatory_ILC2 | minus | Zfp36l1 | TRUE | TRUE | TRUE | TRUE | NA | FALSE |
| Inflammatory_ILC2 | minus | Nmur1 | NA | TRUE | FALSE | NA | NA | FALSE |

Analysis of SMART-Seq2 plate-based scRNA-Seq data. Reads were aligned to mm10 using Kallisto (Bray, N. L., Pimentel, H., Meisted, P. & Pachter, L. Near-optimal probabilistic RNA-seq quantification. Nat Biotechnol 134, 525-527, doi:10.1038/nbt.3519 (2016)) quant, version 0.42.3. The R package tximport (Soneson, C., Love, M. I. & Robinson, M. D. Differential analyses for RNA-seq: transcript-level estimates improve gene-level inferences [version 2; referees: 2 approved]. F1000Research 4, doi:10.12688/f1000research.7563.2 (2016)), version 1.2.0, was used to convert the output to gene counts and traditional TPM values. Because of the variability in read counts per cell across plates, even from the same condition, as well as in the number of genes per cell across conditions, QC was performed for each of eight plates individually in order to remove cells that were outliers with respect to either measure. Out of 752 cells, 606 cells met QC criteria (234 from control, 152 from IL-25 treated mice, and 220 from IL-33 treated mice), and the number of genes per cell in this set ranged from 1,625 to 6,375. Genes that were not expressed with log(TPM)>2.5 in at least two cells were removed from further analysis. Subsequent analysis proceeded analogously to the droplet-based RNA-seq analysis, with parameter settings that reflected both the wider dynamic range of expression and much smaller cell numbers. The minimum ILC signature score required in ILC type assignment was 0.3. Variable genes were identified by running MeanVarPlot with x.low.cutoff=0.1, y.cutoff=1.5, and x.high.cutoff=10, resulting in a set of 519 genes. PCA was performed on the mean-centered expression of variable genes, with 9 PCs chosen for the subsequent clustering analysis (resolution parameter 0.6). RuntSNE was called with the default perplexity value of 30.

The DE analysis for plate-based data followed the structure of the droplet-based analysis but used only logistic regression, with both a condition-based model and a cluster-based model. To rank the results for each model, Applicants created a list of differentially expressed candidate genes that are detected in at least 30% of the cells in one of the groups in the model and have a coefficient for a term of interest with an absolute value at least 1.0 (for condition-based models) or 1.5 (for cluster models) and corresponding FDR-adjusted p-values <1×10⁻⁴ (for condition-based models) or <1×10⁻⁵ (for cluster models). Applicants ranked the candidates by lowest p-value and by largest absolute value of coefficient. The top 40 genes (for condition model) or 3 genes (for cluster model) according to each ranking were reported, with a minimum of 10 genes (for condition model) or 1 gene (for cluster model), if available, selected from each of the set of candidates with positive coefficients and the set with negative coefficients.

To compare plates and droplets, Applicants took as the null set all genes (11,117 genes) detected in both Group A from droplet-based data and in plates. For the PCA comparison, Applicants took the union of the highest and lowest 10 (Group A) or 20 (plates) genes for the PCs used in each analysis (141 genes from droplet-based data, 202 genes from plate-based data, 66 genes in the intersection), and used Fisher's exact test to determine significance (P<3.74×10⁻⁸⁰). For the comparison of differentially expressed genes, Applicants used the previously computed sets of top-ranked DE genes for Group A (219 genes) and for the plate data (72 genes), and again used Fisher's exact test to determine significance of the overlap (P<3.91×10⁻²⁵).

Applicants created a list of candidate differentially expressed genes that contains those genes detected in at least 10% of the cells in one of the conditions and have a coefficient for a term of interest with absolute value at least log(1.2), with corresponding FDR-adjusted P<0.1 (Table 3, 4). The p-value threshold is relaxed compared to droplets, due to lack of power in smaller cell numbers.

TABLE 3
(IL-25)

| | gp.IL25.coeff | gp.IL33.coeff | gp.IL25.pvalue.adj | gp.lL33.pvalue.adj | candidate |
|---|---|---|---|---|---|
| Ms4a4b | 3.49 | 2.44 | 0.070200529 | 0.004529229 | TRUE |
| Epsti1 | 1.79 | 1.72 | 0.070200529 | 0.0044865 | TRUE |
| Tiam1 | −2.1 | −0.828 | 0.060941625 | 0.197952975 | TRUE |
| Lcmt2 | −2.32 | −1.12 | 0.0583245 | 0.058832406 | TRUE |
| Marf1 | 3.17 | 1.15 | 0.055547143 | 0.023928 | TRUE |
| Tet3 | 1.62 | 1.32 | 0.05518395 | 0.006605125 | TRUE |
| Irak3 | 1.76 | 0.157 | 0.036116325 | 0.897030621 | TRUE |
| Pip5k1c | −2.36 | −0.697 | 0.029956015 | 0.258752199 | TRUE |
| Ncoa3 | 2.88 | 1.22 | 0.02413737 | 0.173189351 | TRUE |
| Dgat2 | 2.05 | 0.513 | 0.02413737 | 0.616403736 | TRUE |
| Picalm | 1.79 | 1 | 0.02171466 | 0.049903316 | TRUE |
| Zcchc10 | −1.82 | −0.914 | 0.02171466 | 0.076665862 | TRUE |
| Pim2 | 2.28 | 0.0475 | 0.00661011 | 0.986617099 | TRUE |
| Rel | 2.1 | 0.837 | 0.003517416 | 0.173527438 | TRUE |
| Npy1r | −1.6 | −1.03 | 0.075421703 | 0.035375568 | TRUE |
| Sipa1 | 1.56 | 1.34 | 0.070200529 | 0.006605125 | TRUE |
| Atrx | 1.5 | 0.485 | 0.070200529 | 0.460472954 | TRUE |
| Plcb4 | 1.5 | 0.668 | 0.070200529 | 0.283775088 | TRUE |
| Akirin1 | 1.61 | 0.632 | 0.06388776 | 0.313364769 | TRUE |
| Snrk | 1.55 | 0.356 | 0.05518395 | 0.634918159 | TRUE |
| Ctbp1 | −1.88 | −0.324 | 0.090749659 | 0.737612653 | TRUE |
| Mtfmt | −2.15 | −1.57 | 0.089521326 | 0.057100909 | TRUE |
| Clec2i | 1.98 | 0.974 | 0.085670786 | 0.23789802 | TRUE |
| Rdh1 | −2.49 | −1.91 | 0.082137462 | 0.061611843 | TRUE |
| Gas7 | 1.67 | 0.482 | 0.075421703 | 0.457035981 | TRUE |
| Stat1 | 1.88 | 1.23 | 0.070200529 | 0.026525447 | TRUE |
| Top1 | 1.5 | 0.863 | 0.085670786 | 0.127700851 | TRUE |
| Pik3cd | 1.43 | 0.807 | 0.085670786 | 0.155738276 | TRUE |

TABLE 4
(IL-33)

| | gp.IL25.coeff | gp.IL33.coeff | gp.IL25.pvalue.adj | gp.IL33.pvalue.adj | candidate |
|---|---|---|---|---|---|
| Igfbp4 | −2.9 | −2.3 | 0.328922287 | 0.006043579 | TRUE |
| Shank2 | −3.19 | −3.62 | 0.131681688 | 0.004462572 | TRUE |
| Lilrb4 | 0.943 | 2.11 | 0.490899552 | 7.54E−05 | TRUE |
| Zbp1 | 0.971 | 2.38 | 0.514614181 | 2.45E−06 | TRUE |
| Socs1 | −1.36 | −1.32 | 0.141732614 | 0.00574272 | TRUE |
| Ms4a6b | 3.22 | 2.02 | 0.078159553 | 0.0044865 | TRUE |
| Lgals3bp | 0.804 | 1.95 | 0.709372103 | 0.0044865 | TRUE |
| Epsti1 | 1.79 | 1.72 | 0.070200529 | 0.0044865 | TRUE |
| Ffar2 | 1.36 | 1.38 | 0.120999545 | 0.0044865 | TRUE |
| 4632428N05Rik | 1.28 | 1.58 | 0.323859934 | 0.004262175 | TRUE |
| Phf11a | 1.52 | 1.98 | 0.310827302 | 0.003181964 | TRUE |
| Gp49a | 1.04 | 1.62 | 0.386641463 | 0.003181964 | TRUE |
| Gskip | 0.645 | 1.72 | 0.698543226 | 0.001453626 | TRUE |
| Ly6a | 0.412 | 1.67 | 0.833704733 | 0.000830501 | TRUE |
| Slfn2 | 0.267 | 1.6 | 0.919977774 | 0.000830501 | TRUE |
| Anxa2 | 0.693 | 1.81 | 0.702267971 | 0.000608882 | TRUE |
| Trafd1 | 0.993 | 1.81 | 0.469251276 | 0.000608882 | TRUE |
| Lpcat2 | −0.773 | −1.56 | 0.515901437 | 0.000608882 | TRUE |
| Cirbp | −1.41 | −1.67 | 0.104860941 | 0.000608882 | TRUE |
| Gbp6 | 0.949 | 1.87 | 0.490899552 | 0.000232102 | TRUE |
| Trip13 | −1.91 | −2.7 | 0.539011346 | 0.087578938 | TRUE |
| AI593442 | −2.55 | −3.15 | 0.366414849 | 0.087571546 | TRUE |
| Vdr | −0.748 | 2.04 | 0.912852298 | 0.08751053 | TRUE |
| Oas1a | 0.0294 | 2.09 | 1 | 0.073479685 | TRUE |
| Gm12250 | 3.54 | 3.39 | 0.141732614 | 0.058846186 | TRUE |
| Fbxl21 | 12.2 | −2.6 | 1 | 0.053838 | TRUE |
| Tango6 | −1.32 | −2.16 | 0.515901437 | 0.043629198 | TRUE |
| Bmp2 | −0.856 | −2.03 | 0.776399425 | 0.038648412 | TRUE |
| Dhx58 | 2.3 | 2.39 | 0.197933824 | 0.024040868 | TRUE |
| AA467197 | −0.724 | 2.99 | 0.89766196 | 0.013833375 | TRUE |
| Rab7l1 | 1.18 | 2.2 | 0.565041855 | 0.008507733 | TRUE |
| Isg15 | 2.03 | 3.16 | 0.330584211 | 0.007987729 | TRUE |
| Ctla4 | 1.48 | 2.65 | 0.477699281 | 0.007987729 | TRUE |
| Isg20 | 1.54 | 2.26 | 0.351795037 | 0.007987729 | TRUE |
| Rtp4 | 0.866 | 2.55 | 0.836409396 | 0.006043579 | TRUE |
| Ms4a4b | 3.49 | 2.44 | 0.070200529 | 0.004529229 | TRUE |
| Dkk3 | −0.0269 | 0.959 | 1 | 0.099761925 | TRUE |
| Ddrgk1 | −0.21 | −0.935 | 0.931580949 | 0.099761925 | TRUE |
| Commd10 | −0.867 | −1.3 | 0.609654107 | 0.099574984 | TRUE |
| Tap2 | 0.406 | 1.07 | 0.867316214 | 0.097630755 | TRUE |
| Plac8 | 0.268 | 1.62 | 0.951245067 | 0.096828892 | TRUE |
| 1700113H08Rik | −0.86 | −1.32 | 0.655772517 | 0.096828892 | TRUE |
| Nsmaf | 1.41 | 0.914 | 0.129872368 | 0.09630258 | TRUE |
| Mrpl52 | 0.158 | 0.944 | 0.961345695 | 0.095769519 | TRUE |
| Gpr108 | 0.205 | 0.909 | 0.937163286 | 0.09321466 | TRUE |
| Gigyf1 | −0.844 | −0.966 | 0.454729268 | 0.09321466 | TRUE |
| Plekha5 | −1.01 | −1.23 | 0.530484418 | 0.092360521 | TRUE |
| AW112010 | 0.787 | 0.998 | 0.587991809 | 0.090316471 | TRUE |
| C1qbp | −0.742 | −1.08 | 0.620445671 | 0.090316471 | TRUE |
| Abtb2 | 0.292 | 1.34 | 0.950836855 | 0.090025164 | TRUE |
| Ifngr2 | −0.761 | −0.881 | 0.520644716 | 0.090025164 | TRUE |
| Fhl2 | −1.07 | −0.933 | 0.323945367 | 0.090025164 | TRUE |
| Idh2 | −0.56 | −1.43 | 0.825779497 | 0.090025164 | TRUE |
| Mrps18a | −1.24 | −1.11 | 0.301448306 | 0.089129799 | TRUE |
| Adsl | −1.77 | −0.98 | 0.120999545 | 0.088826678 | TRUE |
| Bcl3 | 1.33 | 1.42 | 0.428888907 | 0.088823636 | TRUE |
| Tradd | 0.964 | 1.29 | 0.534202775 | 0.088823636 | TRUE |
| Itgae | −0.182 | 1.21 | 0.968373267 | 0.088823636 | TRUE |
| Sesn3 | 0.328 | 1.18 | 0.919887997 | 0.088823636 | TRUE |
| Ccng1 | 0.622 | 0.916 | 0.65223303 | 0.088823636 | TRUE |
| Trim30d | 0.527 | 1.14 | 0.825648863 | 0.087564103 | TRUE |
| Ssr3 | 0.609 | 0.999 | 0.712259411 | 0.087564103 | TRUE |
| Phc3 | 0.476 | 0.936 | 0.766387836 | 0.087564103 | TRUE |
| Plxdc2 | −0.906 | −0.903 | 0.419753898 | 0.087564103 | TRUE |
| Sptssa | −0.974 | −0.952 | 0.412315669 | 0.087564103 | TRUE |
| Mrpl53 | −1.87 | −1.26 | 0.120999545 | 0.087564103 | TRUE |
| Mybbp1a | −0.514 | −0.978 | 0.750905063 | 0.084638936 | TRUE |
| Ccdc142 | −1.86 | −1.56 | 0.199126849 | 0.084620819 | TRUE |
| Dhx37 | −0.288 | −0.889 | 0.884634944 | 0.084602571 | TRUE |
| Ccr2 | −1.53 | −1.11 | 0.151175543 | 0.084602571 | TRUE |
| Dock10 | −0.792 | −0.886 | 0.474128889 | 0.083878043 | TRUE |
| Ccdc50 | 1.05 | 0.948 | 0.345810831 | 0.082877891 | TRUE |
| Paxip1 | −1.15 | −0.998 | 0.275200622 | 0.082877891 | TRUE |
| Sntb1 | 1.29 | 1.19 | 0.389063672 | 0.082472426 | TRUE |
| Myo1f | 0.897 | 1.04 | 0.485993669 | 0.082472426 | TRUE |
| H2-T10 | 0.799 | 0.906 | 0.475558017 | 0.080056506 | TRUE |
| Asph | −1.27 | −1.56 | 0.523995802 | 0.080056506 | TRUE |
| Tpm4 | 0.311 | 0.969 | 0.8973 | 0.079984045 | TRUE |
| Zfp110 | −0.396 | −1.24 | 0.885254421 | 0.079984045 | TRUE |
| Lat2 | −0.0259 | 1.05 | 1 | 0.079571887 | TRUE |
| Npnt | 0.056 | 0.949 | 1 | 0.079571887 | TRUE |
| Exosc4 | −0.31 | −0.925 | 0.872264615 | 0.078812281 | TRUE |
| Cox10 | 0.786 | 0.894 | 0.485993669 | 0.077743168 | TRUE |
| Rnf125 | 1.65 | 1.06 | 0.104860941 | 0.076665862 | TRUE |
| Zcchc10 | −1.82 | −0.914 | 0.02171466 | 0.076665862 | TRUE |
| Mrpl40 | −0.837 | −1.08 | 0.518865549 | 0.076564981 | TRUE |
| Gucd1 | −0.0721 | 1.09 | 1 | 0.076166163 | TRUE |
| Ifi35 | 0.307 | 0.95 | 0.894717311 | 0.076166163 | TRUE |
| Bcl2a1d | 0.357 | 0.97 | 0.845361347 | 0.07535918 | TRUE |
| Mlec | 0.715 | 0.999 | 0.591442924 | 0.073543412 | TRUE |
| Mfsd1 | 1.25 | 1.05 | 0.241118045 | 0.072351462 | TRUE |
| Myg1 | −1.33 | −1.26 | 0.328922287 | 0.071926429 | TRUE |
| Il13 | 1.61 | 1.57 | 0.247109843 | 0.0699894 | TRUE |
| Sdc4 | 0.206 | −0.924 | 0.936010617 | 0.069106573 | TRUE |
| Cybrd1 | −0.158 | −1.22 | 0.96501833 | 0.069023077 | TRUE |
| Sema4a | −0.348 | −0.903 | 0.833335843 | 0.068854041 | TRUE |
| Dennd5a | 0.929 | 0.993 | 0.434407143 | 0.068768484 | TRUE |
| Ap3d1 | 0.719 | 0.94 | 0.546182609 | 0.067943704 | TRUE |
| Gba | −0.05 | 0.976 | 1 | 0.067482893 | TRUE |
| Atxn7l3b | 0.557 | 0.957 | 0.702796359 | 0.067482893 | TRUE |
| Socs3 | 1.02 | 1.44 | 0.566715789 | 0.06654975 | TRUE |
| Fus | −0.388 | −0.954 | 0.821454766 | 0.06654975 | TRUE |
| Ddx58 | 0.528 | 0.912 | 0.690537811 | 0.066354958 | TRUE |
| Neu3 | −2.64 | −1.92 | 0.196715769 | 0.066354958 | TRUE |
| Dcaf12 | 0.717 | 0.975 | 0.567597535 | 0.066056553 | TRUE |
| Arf5 | 0.592 | 0.972 | 0.690537811 | 0.066056553 | TRUE |
| Med16 | −0.61 | −0.941 | 0.652753822 | 0.066056553 | TRUE |
| Mkl2 | −1.12 | −0.923 | 0.233085621 | 0.062927532 | TRUE |
| Chst11 | −0.0352 | 1.25 | 1 | 0.062811 | TRUE |
| Mxd1 | 0.448 | 1.05 | 0.804221275 | 0.062811 | TRUE |
| Brix1 | −0.00742 | −1.21 | 1 | 0.062059956 | TRUE |
| Asl | 1.17 | 1.31 | 0.390731792 | 0.061840946 | TRUE |
| Sema4d | 0.548 | 1.28 | 0.806269565 | 0.061840946 | TRUE |
| Gbp4 | 0.318 | 0.935 | 0.865457689 | 0.061840946 | TRUE |
| Dhx35 | −1.63 | −1.51 | 0.295215584 | 0.061840946 | TRUE |
| Lamp2 | 0.692 | 0.996 | 0.591211682 | 0.061740826 | TRUE |
| Rdh1 | −2.49 | −1.91 | 0.082137462 | 0.061611843 | TRUE |
| Sdccag3 | 0.312 | 1.08 | 0.907323767 | 0.060235417 | TRUE |
| Inpp1 | 1.79 | 1.29 | 0.121470313 | 0.058846186 | TRUE |
| Fosb | 0.156 | −0.948 | 0.951245067 | 0.058846186 | TRUE |
| Lcmt2 | −2.32 | −1.12 | 0.0583245 | 0.058832406 | TRUE |
| Sp100 | 0.451 | 1.07 | 0.812545277 | 0.058538143 | TRUE |
| Rab8b | 1.5 | 1.03 | 0.157258763 | 0.057100909 | TRUE |
| Nol9 | −0.775 | −1.02 | 0.510829638 | 0.057100909 | TRUE |
| Mtfmt | −2.15 | −1.57 | 0.089521326 | 0.057100909 | TRUE |
| Hpcal1 | −0.169 | 1.26 | 0.970832752 | 0.055861324 | TRUE |
| Ecm1 | −0.0192 | 1.16 | 1 | 0.055861324 | TRUE |
| Usp36 | −0.645 | −1.09 | 0.670090504 | 0.055861324 | TRUE |
| Lsm6 | −0.616 | −1.12 | 0.768357283 | 0.055861324 | TRUE |
| Soat1 | 0.339 | 1.07 | 0.911016041 | 0.0556326 | TRUE |
| Foxo3 | 2.06 | 1.17 | 0.308917786 | 0.055010352 | TRUE |
| Gnai2 | 0.501 | 1.04 | 0.772683069 | 0.055010352 | TRUE |
| Cblb | 1.08 | 0.969 | 0.290740873 | 0.055010352 | TRUE |
| Smn1 | −1.53 | −1.01 | 0.120999545 | 0.05365299 | TRUE |
| Zc3h12a | −1.14 | −1.12 | 0.429651792 | 0.051606373 | TRUE |
| Tmem33 | 1.18 | 1.08 | 0.310827302 | 0.051407813 | TRUE |
| Inpp4b | −0.752 | −0.999 | 0.520644716 | 0.050799524 | TRUE |
| Atp8b4 | 1 | 1.07 | 0.361882783 | 0.05011516 | TRUE |
| Picalm | 1.79 | 1 | 0.02171466 | 0.049903316 | TRUE |
| Gnl3l | −0.617 | −1.25 | 0.830748338 | 0.049903316 | TRUE |
| Igsf5 | −0.93 | −1.13 | 0.474128889 | 0.04898773 | TRUE |
| Morc3 | 0.662 | 1.05 | 0.65106874 | 0.048717538 | TRUE |
| Tmem229b | 0.199 | 1.01 | 0.942597451 | 0.047806967 | TRUE |
| Fam118a | −0.764 | −0.991 | 0.520644716 | 0.0473575 | TRUE |
| Ndufaf3 | 1.45 | 1.52 | 0.361471564 | 0.047271168 | TRUE |
| Lxn | 0.065 | 1.29 | 1 | 0.047271168 | TRUE |
| Mier3 | −0.901 | −1.41 | 0.591211682 | 0.047271168 | TRUE |
| Syt11 | 0.291 | 0.979 | 0.888359339 | 0.046955301 | TRUE |
| Eva1b | 0.99 | 1.29 | 0.537267316 | 0.045073674 | TRUE |
| Mplkip | −0.607 | −1.13 | 0.702267971 | 0.045073674 | TRUE |
| Nol6 | −1.65 | −1.23 | 0.22835479 | 0.045073674 | TRUE |
| Krit1 | 0.612 | 1.04 | 0.661645455 | 0.043629198 | TRUE |
| E030030I06Rik | −0.87 | −1.01 | 0.438085243 | 0.043629198 | TRUE |
| Cap1 | 1.28 | 1.24 | 0.301448306 | 0.04245761 | TRUE |
| Mgat5 | 1.02 | 1.08 | 0.345290127 | 0.04245761 | TRUE |
| Ctsw | −0.0379 | 1.07 | 1 | 0.04245761 | TRUE |
| Dgka | −0.0503 | 1.02 | 1 | 0.04245761 | TRUE |
| Pin4 | 0.928 | 1.28 | 0.525537288 | 0.040406717 | TRUE |
| St3gal6 | −0.764 | −1.32 | 0.661645455 | 0.040094544 | TRUE |
| Gbp9 | 0.745 | 1.12 | 0.585941803 | 0.039206866 | TRUE |
| Pyhin1 | 0.623 | 1.04 | 0.652753822 | 0.039206866 | TRUE |
| Zfp825 | −1.4 | −1.02 | 0.104860941 | 0.039206866 | TRUE |
| Pml | 1.04 | 1.2 | 0.449297403 | 0.038648412 | TRUE |
| Tmem176a | −0.146 | −1 | 0.954115055 | 0.038648412 | TRUE |
| Xrcc3 | −1.4 | −1.02 | 0.120999545 | 0.038648412 | TRUE |
| Nbn | −0.499 | −1.39 | 0.807775803 | 0.038648412 | TRUE |
| Tnfsf10 | −1.18 | −1.88 | 0.576188312 | 0.038648412 | TRUE |
| Extl3 | −0.16 | −1.19 | 0.955090377 | 0.038491076 | TRUE |
| Irf4 | 1.11 | 1.58 | 0.522128324 | 0.035382894 | TRUE |
| Rab33b | −1.3 | −1.1 | 0.306897549 | 0.035379257 | TRUE |
| Ogfr | 0.578 | 1.12 | 0.715395433 | 0.035371826 | TRUE |
| Gal3st3 | −1.01 | −1.08 | 0.353145297 | 0.035371826 | TRUE |
| Herc6 | 0.579 | 1.21 | 0.726987067 | 0.035100265 | TRUE |
| Chordc1 | 1.4 | 1.09 | 0.129872368 | 0.035100265 | TRUE |
| Adrbk1 | 0.798 | 1.08 | 0.522849808 | 0.035100265 | TRUE |
| Nrip1 | 0.668 | 1.07 | 0.620445671 | 0.035100265 | TRUE |
| Nfil3 | 2.04 | 1.77 | 0.138378795 | 0.034940318 | TRUE |
| Chdh | −0.136 | 1.07 | 0.965100557 | 0.03366614 | TRUE |
| Zfc3h1 | 0.466 | 1.14 | 0.797121248 | 0.032527125 | TRUE |
| H2-T9 | 0.437 | 1.1 | 0.803710077 | 0.032527125 | TRUE |
| Cpsf7 | 0.986 | 1.13 | 0.3988 | 0.032117643 | TRUE |
| Ube2h | 0.767 | 1.05 | 0.519871486 | 0.03194388 | TRUE |
| Taf1d | 1.28 | 1.1 | 0.219781709 | 0.030754234 | TRUE |
| Tap1 | 0.561 | 1.17 | 0.740108759 | 0.030449361 | TRUE |
| Socs2 | −0.923 | −1.33 | 0.486512948 | 0.030449361 | TRUE |
| Mgat1 | 1.24 | 1.21 | 0.247109843 | 0.0293118 | TRUE |
| Ttc19 | 0.891 | 1.44 | 0.526731138 | 0.026842958 | TRUE |
| Hsph1 | 0.237 | 1.26 | 0.943138893 | 0.026842958 | TRUE |
| Naa60 | 0.241 | 1.11 | 0.921741321 | 0.026842958 | TRUE |
| Stat1 | 1.88 | 1.23 | 0.070200529 | 0.026525447 | TRUE |
| Lgals3 | 0.303 | 1.4 | 0.915235942 | 0.025727894 | TRUE |
| Mrp63 | 1.09 | 1.21 | 0.330310435 | 0.025727894 | TRUE |
| Slc25a24 | 0.607 | 1.33 | 0.709372103 | 0.025383081 | TRUE |
| Arhgap1 | 1.34 | 1.12 | 0.141732614 | 0.024367046 | TRUE |
| Nipal1 | 1.61 | 1.74 | 0.193208952 | 0.024094167 | TRUE |
| Ifrd2 | −1.53 | −1.74 | 0.389063672 | 0.024094167 | TRUE |
| Marf1 | 3.17 | 1.15 | 0.055547143 | 0.023928 | TRUE |
| Kit | −0.406 | −1.23 | 0.845361347 | 0.022476058 | TRUE |
| Ppp1r15a | 0.186 | −1.09 | 0.941038405 | 0.0224325 | TRUE |
| Cd247 | 1.16 | 1.52 | 0.465994716 | 0.021571455 | TRUE |
| Trp53inp1 | 1.28 | 1.16 | 0.193208952 | 0.021571455 | TRUE |
| Pfas | −0.546 | −1.41 | 0.754449345 | 0.021217406 | TRUE |
| Il2rb | 0.699 | 1.27 | 0.658568807 | 0.020968484 | TRUE |
| Trim30a | 1.17 | 1.18 | 0.290740873 | 0.020968484 | TRUE |
| Tnfrsf18 | −1.25 | −1.12 | 0.172422353 | 0.018814355 | TRUE |
| Amd1 | −0.74 | −1.75 | 0.655540415 | 0.018814355 | TRUE |
| Crlf2 | 0.355 | 1.16 | 0.851703739 | 0.0187436 | TRUE |
| Ttc39b | 0.546 | 1.16 | 0.713471805 | 0.017643539 | TRUE |
| 1700017B05Rik | −1.49 | −1.5 | 0.344202381 | 0.01743617 | TRUE |
| Inpp5b | 0.937 | 1.22 | 0.429774757 | 0.016798291 | TRUE |
| Rilpl2 | 1.4 | 1.43 | 0.207349054 | 0.016362529 | TRUE |
| Glrx | 0.346 | 1.26 | 0.887397708 | 0.016362529 | TRUE |
| Sbno2 | 1.09 | 1.2 | 0.323859934 | 0.016362529 | TRUE |
| Omd | −1.12 | −1.13 | 0.241118045 | 0.016304598 | TRUE |
| Ddx20 | −1.24 | −1.92 | 0.547870673 | 0.016173556 | TRUE |
| Cst7 | −0.0555 | 1.5 | 1 | 0.016039238 | TRUE |
| Myo1g | 0.257 | 1.31 | 0.933509791 | 0.015333608 | TRUE |
| Map2k4 | 1.89 | 1.64 | 0.120999545 | 0.015300115 | TRUE |
| Serinc5 | 0.779 | 1.44 | 0.600465909 | 0.015300115 | TRUE |
| Mov10 | 1.08 | 1.45 | 0.494819799 | 0.01411752 | TRUE |
| Tmem209 | −0.847 | −1.27 | 0.477990963 | 0.014065784 | TRUE |
| H2-T22 | 0.0651 | 1.27 | 0.997500376 | 0.01339631 | TRUE |
| Impact | −1.46 | −1.54 | 0.193208952 | 0.013331314 | TRUE |
| Parp3 | 0.335 | 1.21 | 0.886033624 | 0.012419152 | TRUE |
| Trim12c | 0.974 | 1.16 | 0.330584211 | 0.012419152 | TRUE |
| Per1 | −0.785 | −1.16 | 0.490743021 | 0.012419152 | TRUE |
| Parp14 | 0.934 | 1.21 | 0.443178659 | 0.01121625 | TRUE |
| Trim34a | 0.632 | 1.29 | 0.684588556 | 0.010822818 | TRUE |
| Pigv | −2.73 | −1.36 | 0.135162911 | 0.010473173 | TRUE |
| Usp18 | 1.1 | 1.6 | 0.507670267 | 0.009898786 | TRUE |
| Pde4b | 0.735 | 1.24 | 0.575491757 | 0.009898786 | TRUE |
| Pydc4 | 1.52 | 1.37 | 0.172422353 | 0.009708492 | TRUE |
| Samd9l | 0.773 | 1.2 | 0.515901437 | 0.009708492 | TRUE |
| Rdh13 | −1.37 | −1.75 | 0.301448306 | 0.009708492 | TRUE |
| Lancl1 | −1.11 | −1.58 | 0.36889 | 0.009700122 | TRUE |
| Slc44a2 | 0.294 | 1.22 | 0.890722932 | 0.009602684 | TRUE |
| Gga1 | 0.739 | 1.33 | 0.572314255 | 0.009070887 | TRUE |
| Endod1 | 0.398 | 1.51 | 0.866595452 | 0.008507733 | TRUE |
| Parp10 | 0.371 | 1.34 | 0.877516699 | 0.008507733 | TRUE |
| Stab1 | 1.24 | 1.65 | 0.349947 | 0.007987729 | TRUE |
| Irf7 | 0.423 | 1.43 | 0.866595452 | 0.007987729 | TRUE |
| Vmp1 | 1.49 | 1.4 | 0.182664643 | 0.007987729 | TRUE |
| Pdcd1 | 1.04 | 1.35 | 0.438085243 | 0.007987729 | TRUE |
| Tmem176b | −0.422 | −1.23 | 0.794836397 | 0.007987729 | TRUE |
| Ahcyl2 | −1.01 | −1.24 | 0.323859934 | 0.007987729 | TRUE |
| AI836003 | −0.781 | −1.26 | 0.520644716 | 0.007987729 | TRUE |
| Nop56 | −0.619 | −1.41 | 0.679264486 | 0.007987729 | TRUE |
| Ppif | −1.14 | −1.91 | 0.452378393 | 0.007987729 | TRUE |
| Il5 | 1.4 | 1.34 | 0.141732614 | 0.007937654 | TRUE |
| 0610031J06Rik | 0.555 | 1.33 | 0.726987067 | 0.00762705 | TRUE |
| Csf1 | −1.01 | −1.61 | 0.502957688 | 0.006669122 | TRUE |
| Sipa1 | 1.56 | 1.34 | 0.070200529 | 0.006605125 | TRUE |
| Tet3 | 1.62 | 1.32 | 0.05518395 | 0.006605125 | TRUE |
| Bcl6 | 1.29 | 1.98 | 0.445256278 | 0.006043579 | TRUE |
| Gng2 | 1.06 | 1.35 | 0.323945367 | 0.006043579 | TRUE |
| Arhgap26 | 0.821 | 1.3 | 0.463235501 | 0.006043579 | TRUE |
| Nlrc5 | 0.93 | 1.3 | 0.387677282 | 0.005815833 | TRUE |
| Zfand6 | 0.659 | 1.38 | 0.65223303 | 0.005797938 | TRUE |
| Grn | −0.326 | 1.55 | 0.904752946 | 0.005346413 | TRUE |
| Phf11b | 1.38 | 1.89 | 0.368386927 | 0.005110709 | TRUE |
| Acot7 | 0.807 | 1.78 | 0.690230769 | 0.004649645 | TRUE |
| Npc2 | 0.143 | 1.53 | 0.971845693 | 0.0044865 | TRUE |

To compare plates and droplets, Applicants took as the null set all genes (11,132 genes) detected in both Group A from droplet-based data and in plates. For the differentially expressed genes, Applicants used those genes that met the “candidate” selection requirements in each group, intersected with the null set (for IL-25, 1,166 genes in Group A and 35 genes in plates, with 17 in the intersection; for IL-33, 1,489 genes in Group A and 35 in plates, with 24 in the intersection) and computed Fisher's exact test to determine significance.

Data availability. The data discussed in this application have been deposited in NCBI's Gene Expression Omnibus and are accessible through GEO Series accession number GSE102299.

### Example 2—CGRP Negatively Regulates Alarmin-Driven ILC2 Responses ILC2s Express the CGRP Receptor Subunits Ramp1 and Calcr1

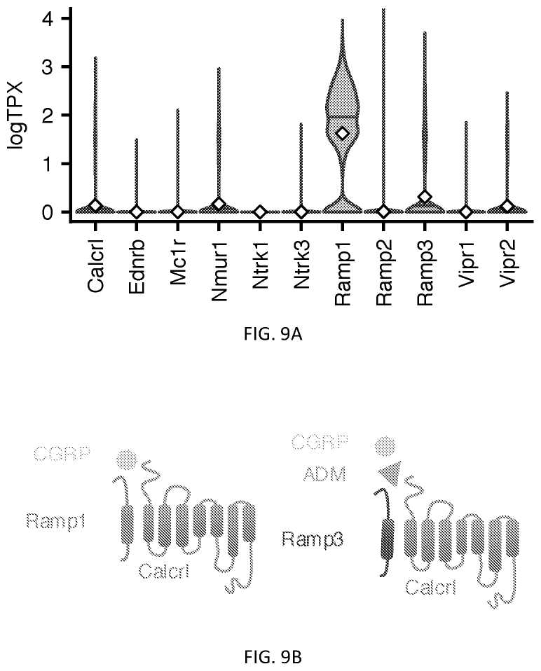

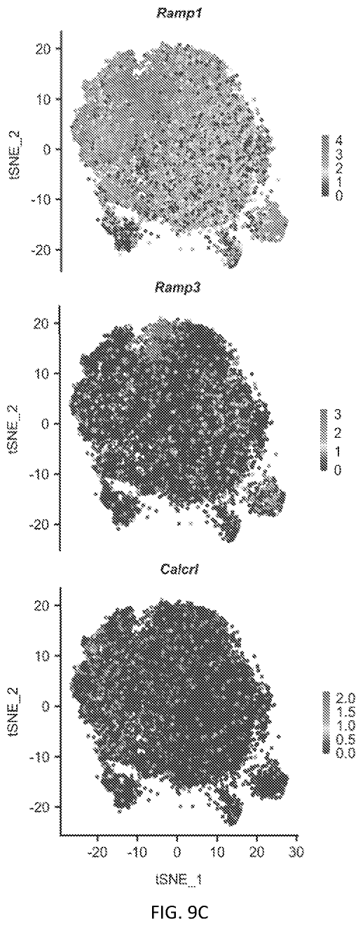

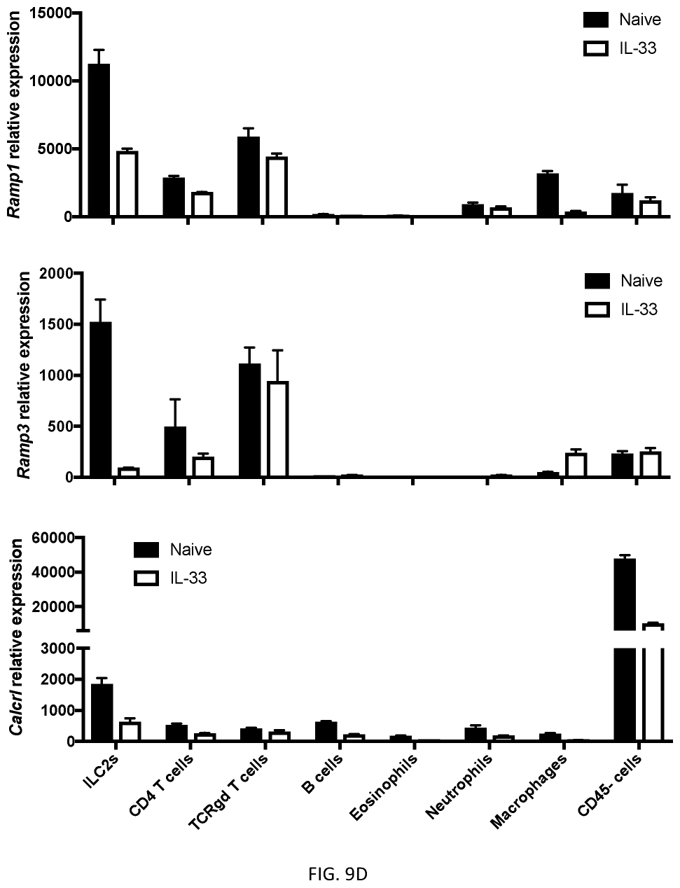

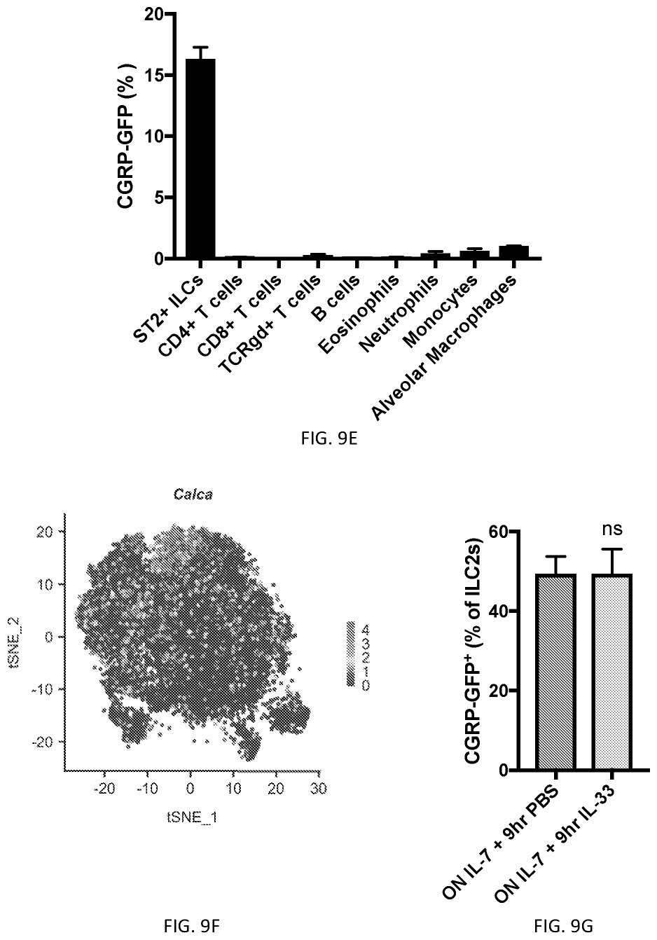

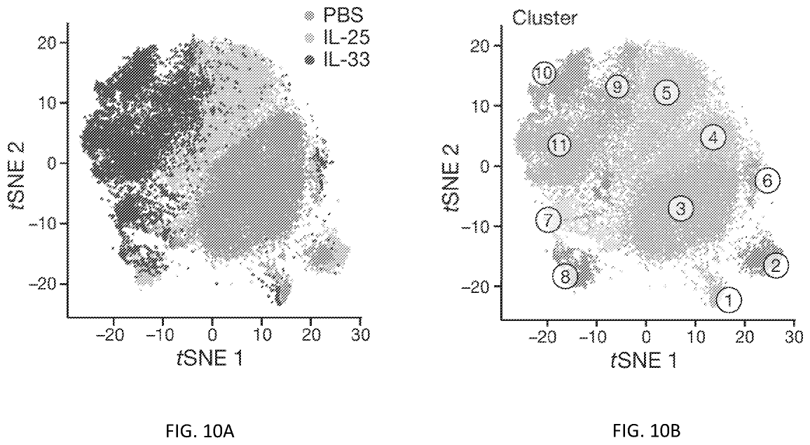

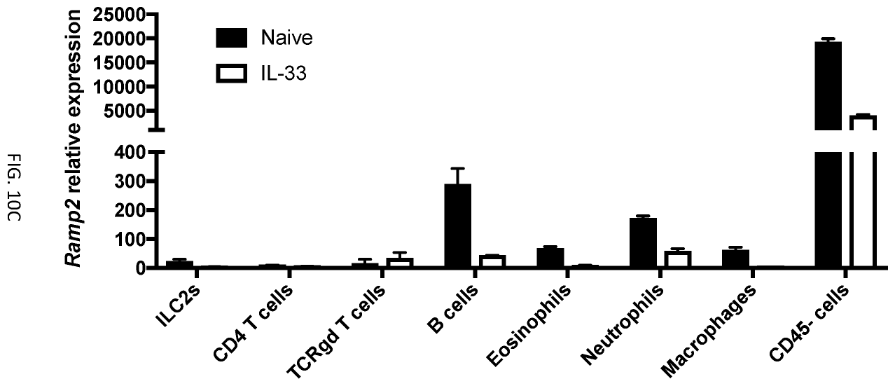

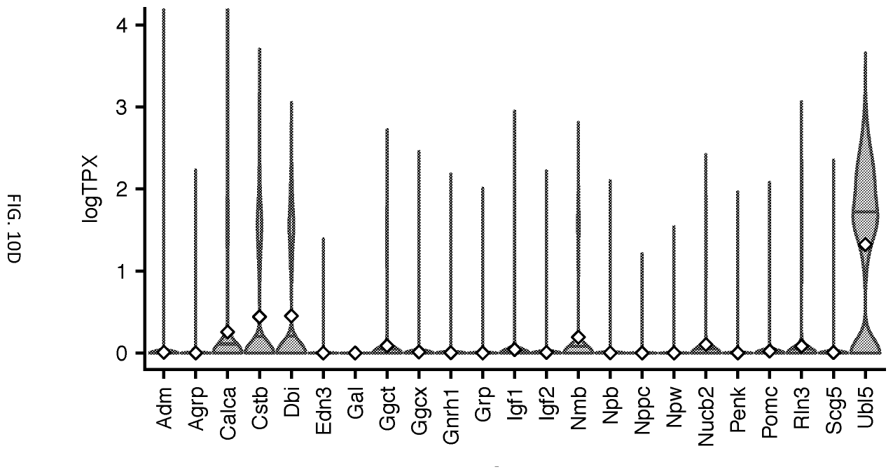

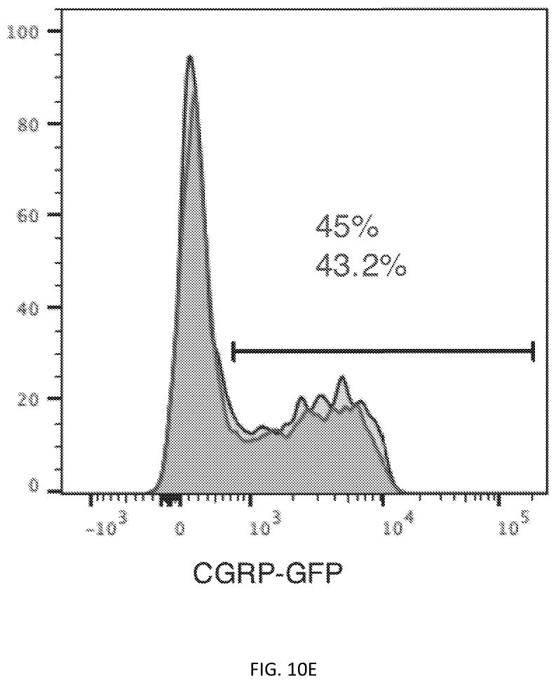

To identify putative neuroimmune interactions that may modify ILC2-mediated responses, Applicants analyzed the expression of a set of neuropeptide receptors from a previously generated scRNA-seq atlas of steady-state lung ILCs (Wallrapp et al., 2017). Consistent with previous studies, ILC2s expressed Vipr2 and Nmur1, the receptors for VIP and NU, respectively (FIGS. 9A and 10). While most other neuropeptide receptors were either undetectable or minimally expressed (e.g., Ntrk1, Ntrk3, and Mc1r; FIG. 9A), Ramp1, Ramp3, and Calcrl were expressed at significant levels in a substantial proportion of cells (FIG. 9A). Calcrl encodes a gene that, in a complex with Ramp1, forms a G protein-coupled receptor which binds the neuropeptide calcitonin gene-related peptide (CGRP), while the combination of Ramp3 and Calcrl form the receptor for adrenomedullin (ADM) (FIG. 9B), and can also bind CGRP, albeit with lower affinity (Russell et al., 2014).

Applicants further determined which cell subsets expressed Ramp1, Ramp3 and Calcrl during steady-state and airway inflammation in the scRNA-seq data set, which contains ILCs isolated from mice treated with either IL-33 or IL-25, or control mice (FIGS. 9C and 10A,B) (Wallrapp et al., 2017). Both Ramp1 and Calcrl were expressed by lung-resident ILCs from all conditions, although Calcrl levels were lower than Ramp1 (FIG. 9C). In contrast, Ramp3 was primarily highly expressed by a small, discrete subset of ILCs, with additional scattered expression in other ILCs (FIG. 9C).

Applicants validated these results with quantitative real-time PCR (qPCR) of Ramp1, Ramp3, and Calcrl on sort-purified lung-resident cell populations isolated from either naïve or IL-33-treated mice. All three genes were highly expressed in naïve ILC2s, and their expression was reduced in ILC2s from IL-33 treated mice, indicating that ILC2s may downregulate the receptor in response to IL-33 driven inflammatory responses (FIG. 9D). Other immune cell populations and CD45− stromal cells also expressed varying levels of Ramp1, Ramp3 and Calcrl, indicating that these neuropeptide receptors are not exclusively expressed by ILC2s in the lung. Nevertheless, expression of Ramp1 and Calcrl were highest in ILC2s when compared to the other immune cell populations (FIG. 9D), suggesting that ILC2s may be a particularly CGRP-responsive cell type. Applicants also confirmed that ILCs express very little Ramp2, which also binds adrenomedullin, consistent with the scRNA-seq data (FIG. 10C).

### Lung ILC2s Express the Neuropeptide CGRP

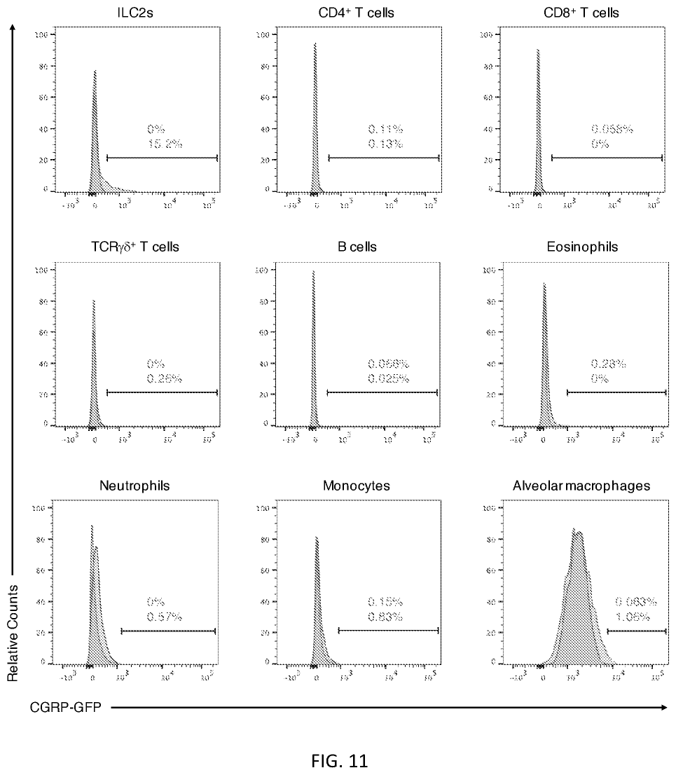

Applicants next determined the cellular source of CGRP in the lung. While neurons and neuroendocrine cells are well known to express CGRP (Branchfield et al., 2016; Chiu et al., 2013; Sui et al., 2018), Applicants also tested if CGRP is expressed at steady state in different lung-resident immune cell populations using mice that express GFP under control of the CGRP promoter (CGRP-GFP reporter mice). Most lung-resident immune cell populations, including myeloid, B, and T cells, from the mice lungs showed minimal (<1%) CGRP expression, but ˜15% of lung resident ST2⁺ ILC2s expressed CGRP at steady state (FIGS. 9E and 11). Consistently, Calca, the gene that encodes CGRP, was largely co-expressed in the same subset of lung ILCs that also highly expressed Ramp3 in scRNA-seq (FIG. 9F). Thus, while most lung-resident ILC2s express the receptor for CGRP, a sub-population of ILC2s also express CGRP itself. Moreover, ILCs expressed several other genes encoding neurotransmitters, including Ub15, which encodes Beacon, and neuromedin B (Nmb), both of which have been implicated in regulating organismal metabolism. However, of these neuropeptides, Calca was the only one for which ILCs also expressed the receptor (FIG. 10D), suggesting a feedback loop involving CGRP and its receptor may regulate ILC2 function.

To further characterize what regulates the expression of CGRP in steady state and activated ILC2s, Applicants isolated ST2⁺ lung ILCs from CGRP-GFP reporter mice and cultured them in vitro with IL-7 or IL-7+IL-33. While in vitro culture increased the frequency of CGRP-GFP+ILCs compared to what Applicants observed in vivo, stimulation with IL-33 did not further increase the frequency of CGRP-GFP⁺ ILC2s (FIGS. 9G and 10E). Taken together, the data show that ILCs uniquely express both chains of the CGRP receptor as well as CGRP itself, suggesting that this pathway may play a key role in regulating ILC responses, potentially in an autocrine or paracrine manner.

### CGRP Negatively Regulates ILC2 Responses Driven by IL-33 and IL-25 In Vitro

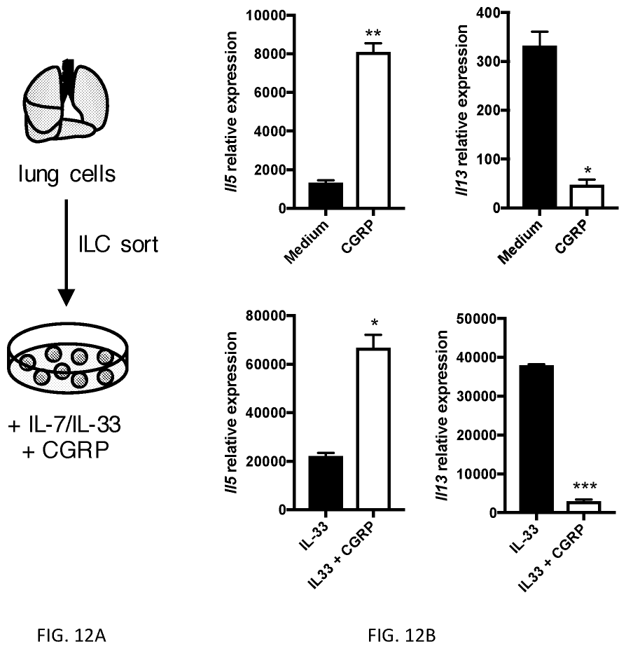

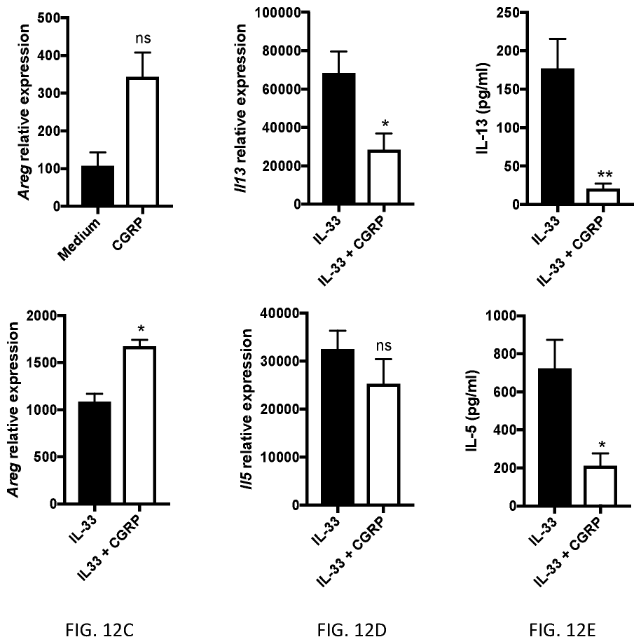

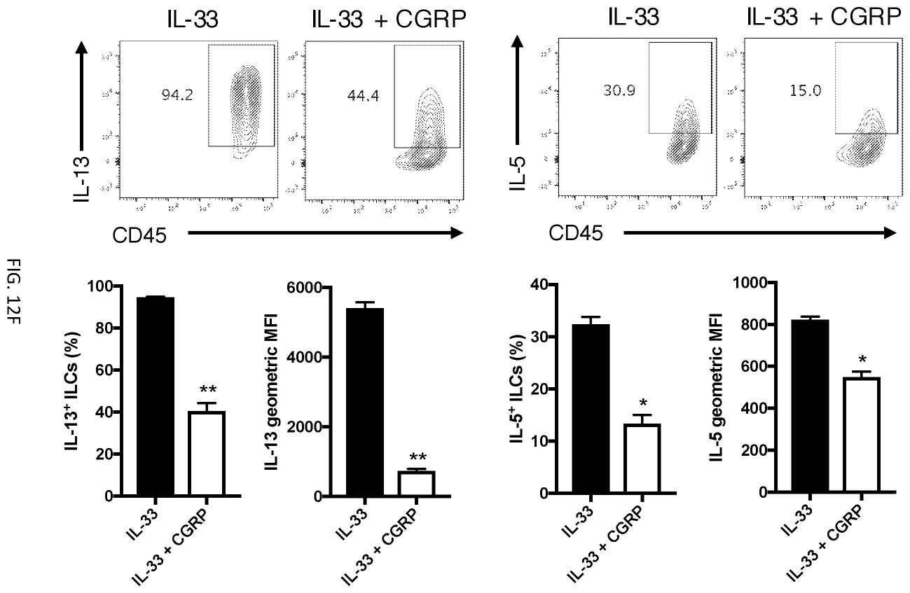

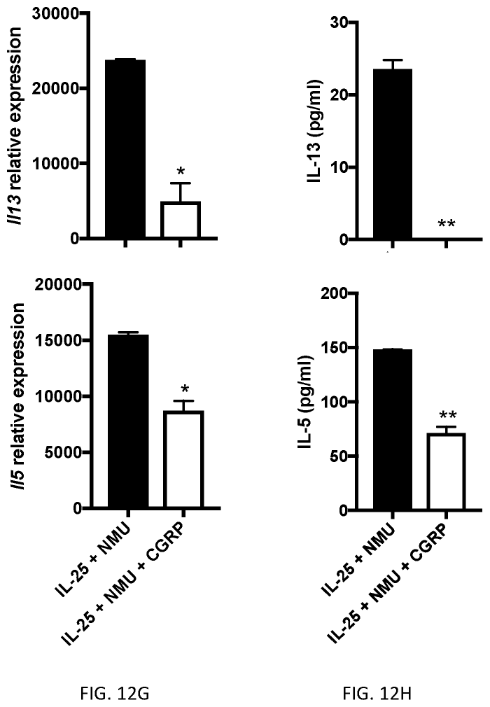

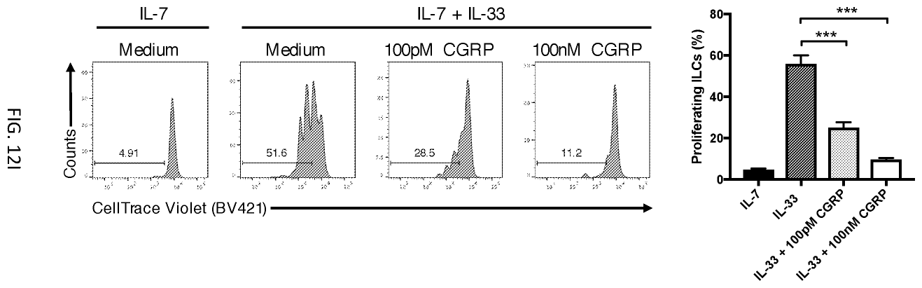

To investigate how CGRP affects ILC2 function, Applicants first examined its effects on ILC2s in vitro, either alone or together with IL-33, an alarmin which activates ILC2s and induces their proliferation. Applicants sort-purified ST2⁺ ILC2s from the lungs of naïve C57BL/6 wild-type mice, cultured them in vitro with IL-7 overnight, and then treated them with either PBS (control) or IL-33 in the presence or absence of CGRP (FIG. 12A). A recent report demonstrated that CGRP enhanced IL-5 and amphiregulin (Areg) production by ILC2s (Sui et al., 2018), inferring it promotes ILC2 activation. Indeed, after 6 hours, ILC2s cultured with CGRP had upregulated expression of Il5 compared to ILC2s cultured with IL-7 alone (FIG. 12B, top) and showed a trend of increased expression of amphiregulin (FIG. 12C, top).

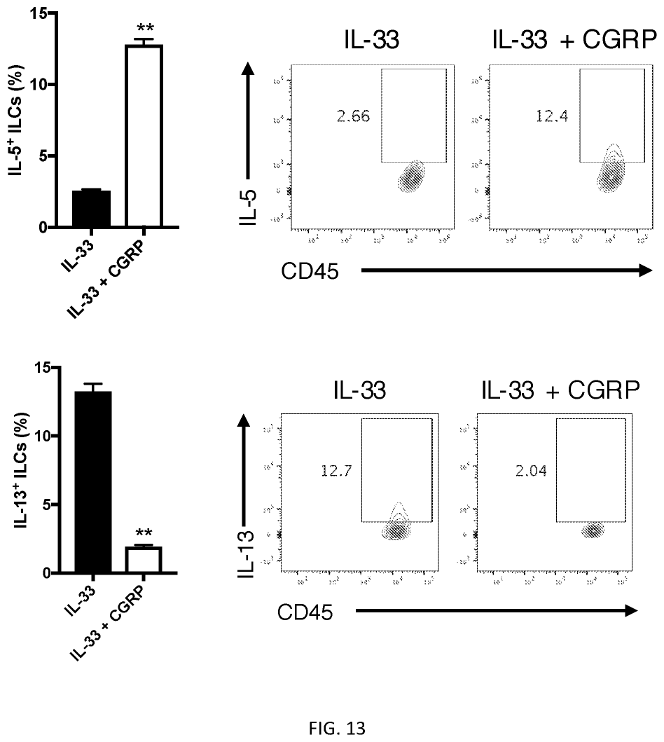

Surprisingly, however, CGRP downregulated expression of the pro-inflammatory cytokine Il13, indicating that CGRP may have a more nuanced role in regulating ILC2 responses than initially appreciated (FIG. 12B). A similar pattern was seen in the presence of IL-33 stimulation: Il13 expression was significantly decreased and Il5 and Areg expression significantly increased compared to cells cultured with IL-33 alone (FIG. 12B,C, bottom). Applicants observed similar results at the protein level by intracellular cytokine staining: short-term treatment with CGRP+IL33 induced IL-5 protein production compared to IL-33-alone (FIG. 13A), whereas the frequency of IL-13-positive ILC2s was significantly reduced (from ˜13% to 2%) (FIG. 13B). Thus, CGRP treatment rapidly alters expression of three key effector cytokines produced by ILC2s, induced expression of both IL-5 and Areg, while repressing IL-13.

Importantly, the impact of CGRP on IL33-induced changes in cytokine expression change over time, as Applicants observed when culturing sort-purified lung ILCs with IL-33 in the presence or absence of CGRP for 3 days. Il13 mRNA expression (FIG. 12D) and IL-13 protein production (FIG. 12E) remains repressed by CGRP at 3 days, similar to the observations at 6 hours, with lower frequency of IL-13⁺ ILCs and less IL-13 produced per cell (FIG. 12F). However, in contrast to short-term CGRP treatment, at 3 days IL-5 production was significantly reduced at both the mRNA and protein levels (FIG. 12D,E), with significant decreases in both the frequency of IL-5-producing ILCs and the amount of IL-5 expressed per cell (FIG. 12F). Thus, over time CGRP inhibits IL33-induced production of both IL-5 and IL-13.

CGRP similarly inhibited the response driven by IL-25 and NMU and IL-25, which recently were shown to synergize to promote type 2 cytokine production in lung ILC2s (Wallrapp et al., 2017). Applicants cultured sort-purified lung ILCs with IL-25+NMU in the presence or absence of CGRP for three days. CGRP significantly reduced type 2 cytokine production at both the mRNA and protein level, compared to controls (FIG. 12G,H), indicating that CGRP inhibits the production of pro-inflammatory type 2 cytokines by ILC2s in response to two distinct stimuli.

CGRP also suppressed ILC2 proliferation, which is potently induced by IL-33 in vitro (Moro et al., 2010; Neill et al., 2010). Applicants labeled sort-purified lung ILCs with CellTrace Violet and cultured them with IL-7 or IL-7+IL-33 for 3 days, the latter in the presence or absence of CGRP. As expected, absent CGRP, IL-33 induced significant ILC2 proliferation compared to IL-7 alone (FIG. 12I). However, addition of CGRP strongly inhibited IL-33-induced proliferation of ILCs in a dose-dependent manner (FIG. 12I). While approximately 55% of IL-33-activated ILCs divided at least once, less than 10% of IL-33-activated ILCs proliferated in the presence of 100 nM CGRP. Overall, CGRP negatively regulates alarmin driven ILC2 proliferation and production of pro-inflammatory type 2 cytokines, while promoting expression of Areg.

iILC2s do not Express the Receptor to CGRP and are not Inhibited by it

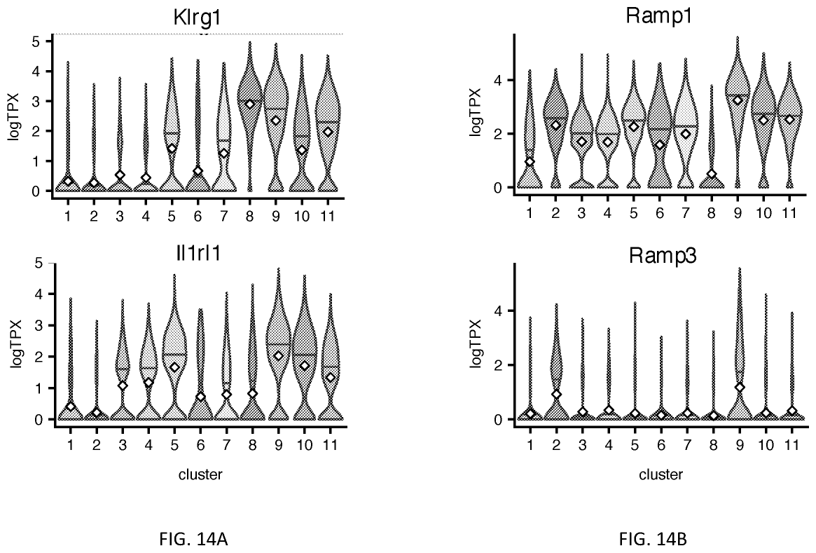

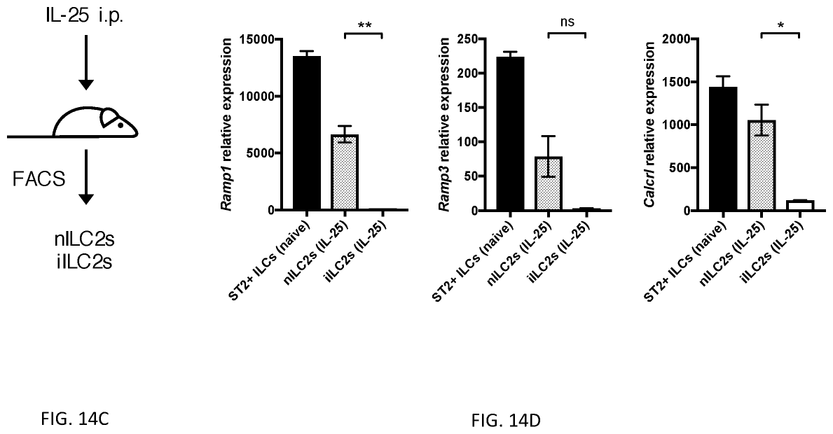

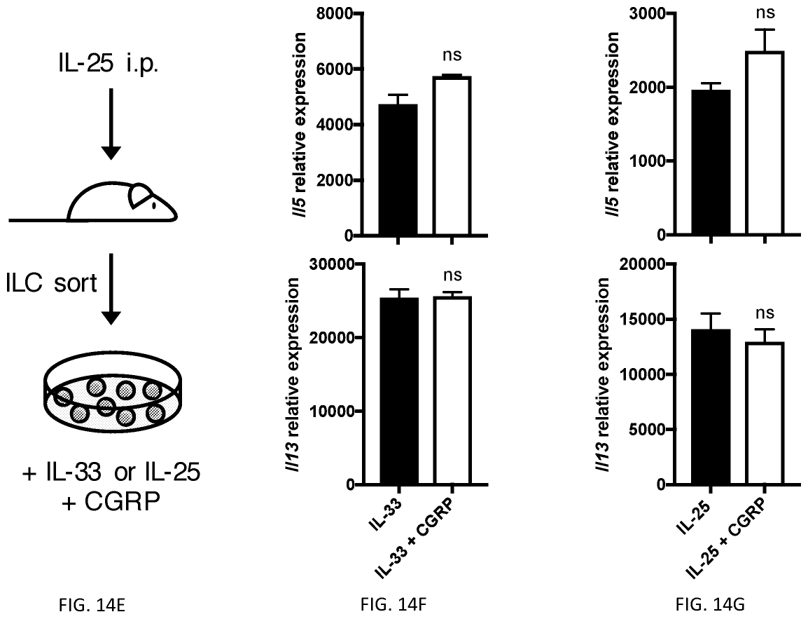

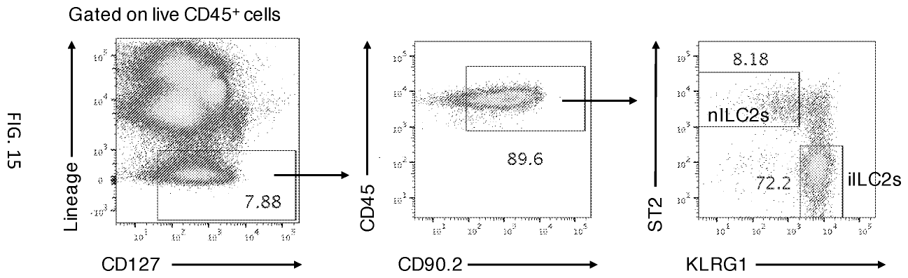

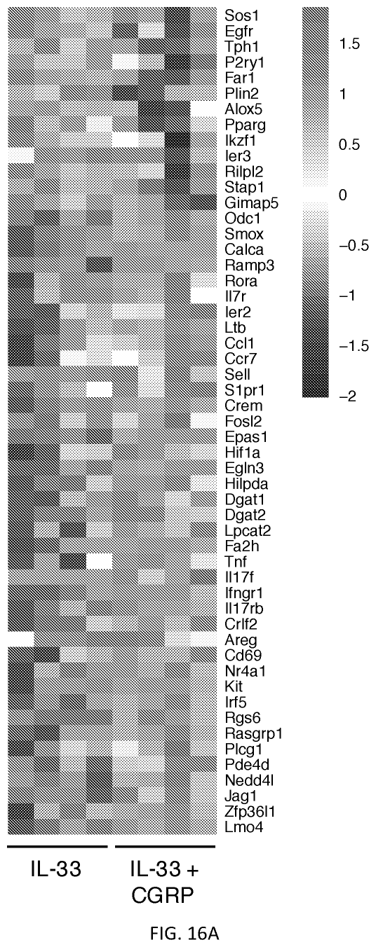

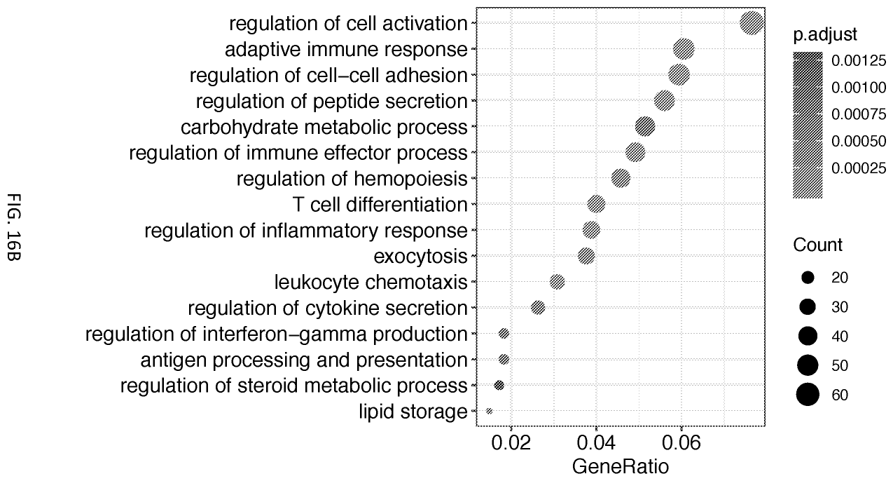

Recent work has demonstrated that during helminth infection a distinct population of inflammatory ILC2s (iILC2s) arises in the intestines and migrates to the lung and other organs, where it plays an important role in host defense (Huang et al., 2018). Cluster 8 in the scRNA-seq data of lung-resident ILCs (FIG. 10B) expresses key marker genes of iILC2s (Huang et al., 2015; Huang et al., 2018; Wallrapp et al., 2017) (e.g., Klrg1, FIG. 14A).

Importantly, Cluster 8 cells had significantly reduced expression of Ramp1 compared to other ILC2s, and minimal expression of Ramp3 (FIG. 14B), suggesting that iILC2s may not be inhibited by CGRP, given they lack expression of the receptor. To confirm that iILC2s had reduced Ramp1 expression, Applicants sort-purified lung-resident natural ST2+ILCs (nILC2s) and inflammatory KLRG1^{Hi} ST2⁻ ILCs (iILC2s) from wild-type mice treated intraperitoneally (i.p.) with IL-25 for three consecutive days. Ramp1 and Calcrl were both expressed at extremely low levels in iILC2s compared to nILC2s (by qPCR, FIG. 14C,D and S4), indicating that the CGRP receptor is primarily expressed on ST2⁺ nILC2s.

Applicants confirmed that lower Ramp1 expression impacts the ability of iILC2s to respond to CGRP. To this end, Applicants induced iILC2s by i.p. injection of IL-25 on three consecutive days and then cultured sort-purified iILC2s with either IL-33 or IL-25 in the presence or absence of CGRP (FIG. 14E). iILC2s cultured with or without CGRP had no differences in Il5 and Il13 expression in response to either IL-25 or IL-33. Thus, while ST2⁺ nILC2s are strongly inhibited by CGRP, it does not inhibit type 2 cytokine expression by iILC2s which do not express its receptor (FIG. 14F,G).

### CGRP Induces a Regulatory Gene Expression Program in ILC2s

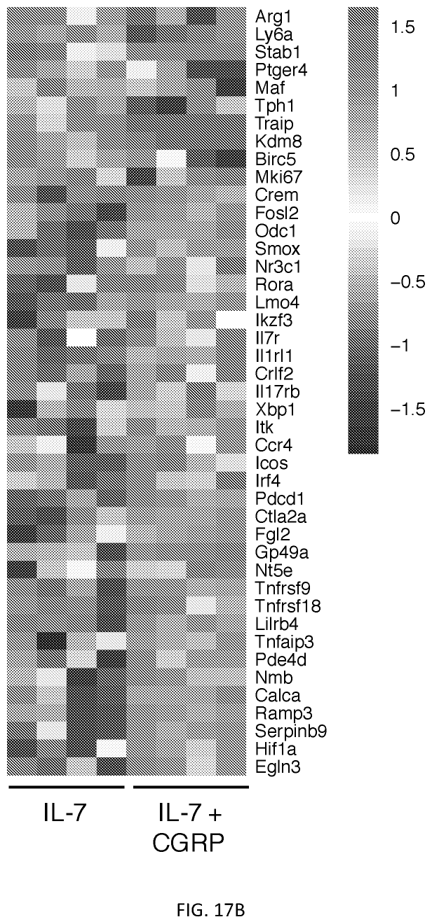

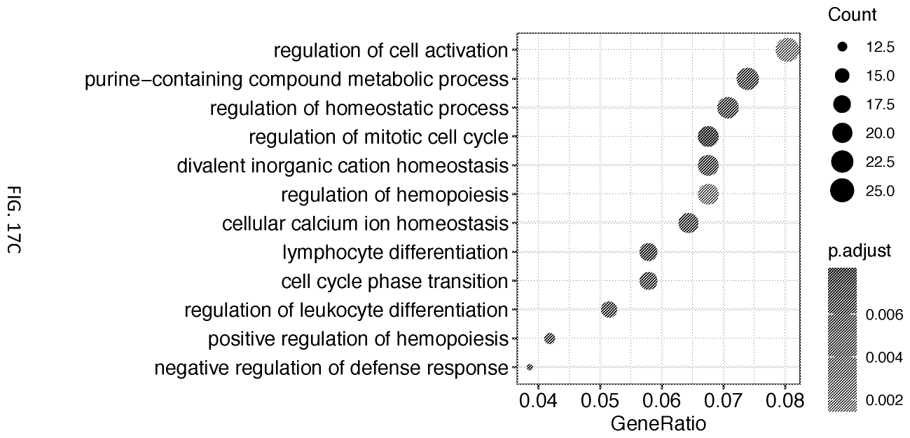

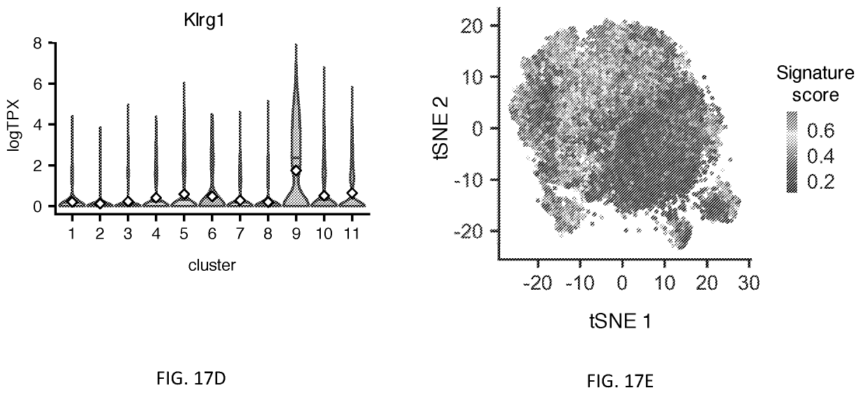

To uncover possible mechanisms underlying the inhibitory effects of CGRP on lung ILC2s, Applicants compared expression profiles of lung ILC2s cultured with CGRP, IL-33 or both for 3 days. First, CGRP dramatically altered the transcriptional response of ILC2s to IL-33 (>900 differentially expressed genes; P<0.05, fold change >1.5, Methods). CGRP actively promoted a distinct transcriptional state, with two thirds (635 of 946) of genes up-regulated in ILCs stimulated in its presence (FIG. 17). These included genes known to be downstream of cAMP-mediated signaling, including Crem and Fosl2, the critical ILC2 transcription factor Rora, Il7r, a pro-survival growth factor receptor, and Il17rb and Crlf2, which encode the unique receptors for the alarmins IL-25 and TSLP, respectively (FIG. 16A). It also induced expression of genes associated with polyamine metabolism (e.g., Odc1, Smox), immune effector responses (e.g., Tnf, Areg, Il17f), lymphocyte activation (e.g., Nr4a1, Cd69), and hypoxia (e.g., Hif1a, Egln3, Epas1) (FIG. 16A). Both Ramp3 and Calca were upregulated by CGRP, which Applicants confirmed by qPCR (FIG. 17A), indicating that CGRP may modulate its own expression in ILC2s. The induced genes were enriched for leukocyte chemotaxis (e.g., Sel1, S1p1r, Ccr7), lipid storage (e.g., Dgat1, Dgat2, Hilpda), and regulation of cell activation (e.g., Icos, Tnfaip3, Ikzf1) (FIG. 16A,B, Table SX, Methods).

Notably, in the presence of IL-33 CGRP upregulated genes associated with negative regulation of effector T cell responses, including several often associated with regulatory CD4 T cells (FIG. 16C, Methods). These included the cell surface molecules Pdcd1 (PD-1), Havcr2 (Tim-3), Lilrb4, Entpd1 (CD39), Tnfrsf18 (GITR), and the transcription factors FoxP3, Nfil3, and Nr3c1 (the glucocorticoid receptor) and the soluble mediator Fgl2. In contrast, genes associated with effector ILC2 responses, such as Il13 and Arg1 were significantly down-regulated by CGRP (FIG. 16C). Thus, CGRP may inhibit ILC2 function by inducing cell surface molecules and transcription factors associated with T cell regulation or exhaustion/dysfunction.

The regulatory program was induced by CGRP even in the absence of IL-33 (FIG. 17B,C). Although CGRP treatment alone resulted in fewer differentially expressed genes (331 vs. 946 genes), one third (112) were shared with those from CGRP+IL-33 treatment and included many of the negatively regulatory genes, including Pdcd1, Lilrb4, Fgl2, Nr3c1, and Tnfrsf18 (FIG. 17B,C). CGRP treatment alone also down-regulated the expression of genes associated with cell cycle progression (e.g., Mki67, Birc5, Kdm8), consistent with its inhibition of alarmin-driven proliferation by CGRP (FIG. 12I, FIG. 17B). Thus, CGRP induces changes in ILC2 gene transcription, inducing genes known to negatively regulate lymphocyte effector function and promote T cell exhaustion, and inhibiting genes that promote ILC2 proliferation and effector function.

The in vitro CGRP-induced program is enriched in a subset of ILC2s in vivo that express Calca and Ramp3. To assess the program in vivo, Applicants generated a gene signature of the CGRP response from the in vitro profiles (Methods) and scored each of the lung resident ILC2 scRNA-Seq profiles by this signature (FIG. 16D, Methods). The signature was most prominent in Cluster 9 cells (FIG. 16D, 10B, 13B, 17D). This cluster is chiefly composed of ILCs from IL-33-treated mice and its cells express Calca and Ramp3 at higher levels (FIG. 14B), and the enrichment is maintained even when excluding Calca and Ramp3 from the scored signature (FIG. 17E, Methods). Thus, Applicants hypothesize that these ILCs may represent a population exposed to endogenous CGRP.

### In Vivo Administration of CGRP Limits Alarmin-Induced Airway Inflammation

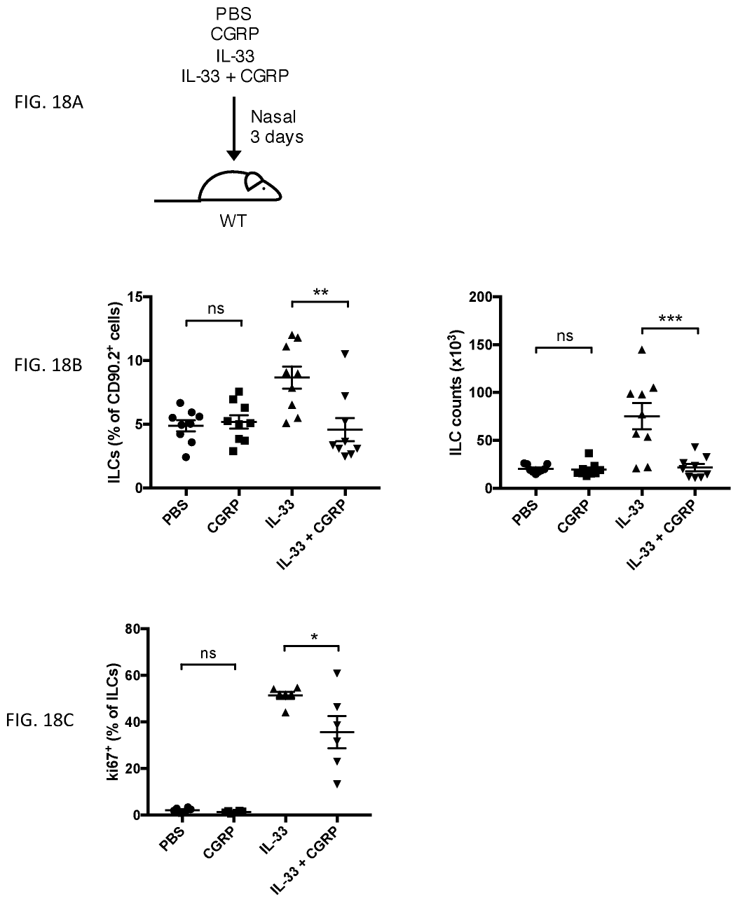

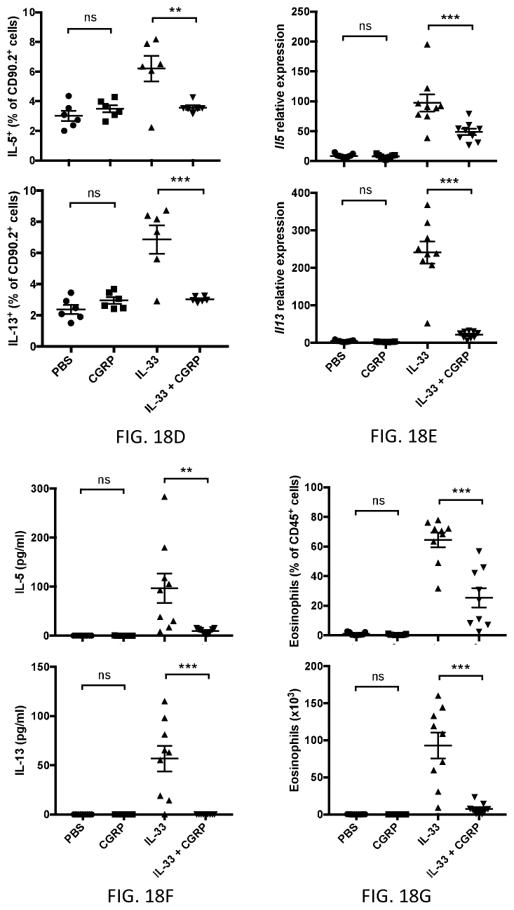

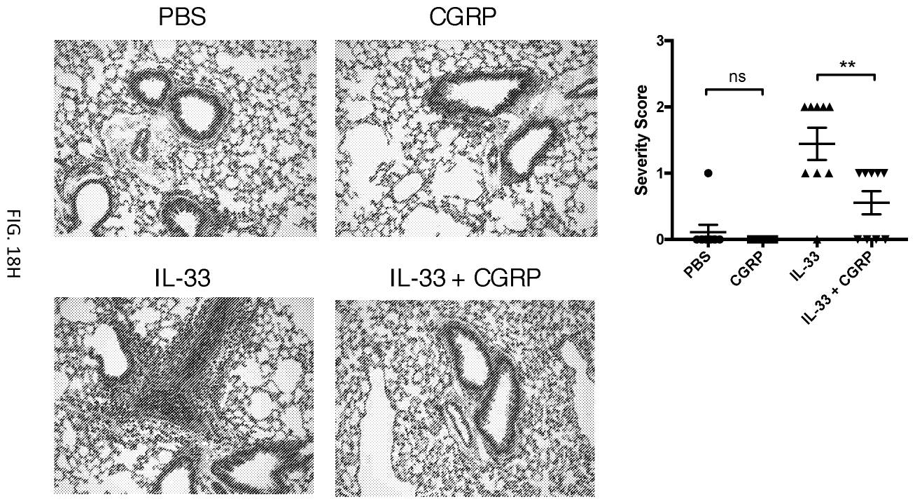

To test the in vivo role of CGRP, Applicants next analyzed a mouse model of ILC-driven acute airway inflammation. Applicants treated wild-type mice intranasally with either PBS, CGRP, IL-33 or IL-33+CGRP for three consecutive days and assessed the severity of airway inflammation one day after the last treatment (FIG. 18A).

CGRP restrained IL-33-induced ILC proliferation. While CGRP treatment alone did not alter the frequency or total number of lung-resident ILCs compared to PBS-treated mice, ILC frequencies and numbers were significantly reduced in mice that received IL-33+CGRP compared to IL-33 alone, indicating that CGRP inhibits IL-33-induced expansion of ILCs in vivo (FIG. 18B). Indeed, intracellular staining showed decreased frequencies of ILCs with the proliferation marker Ki67 in mice treated with IL-33+CGRP compared to those treated with IL-33 alone (FIG. 18C).

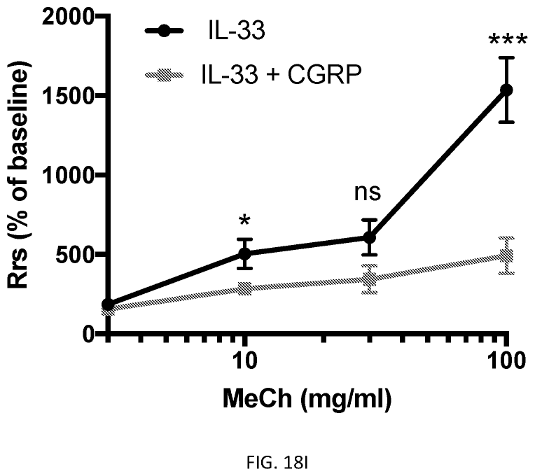

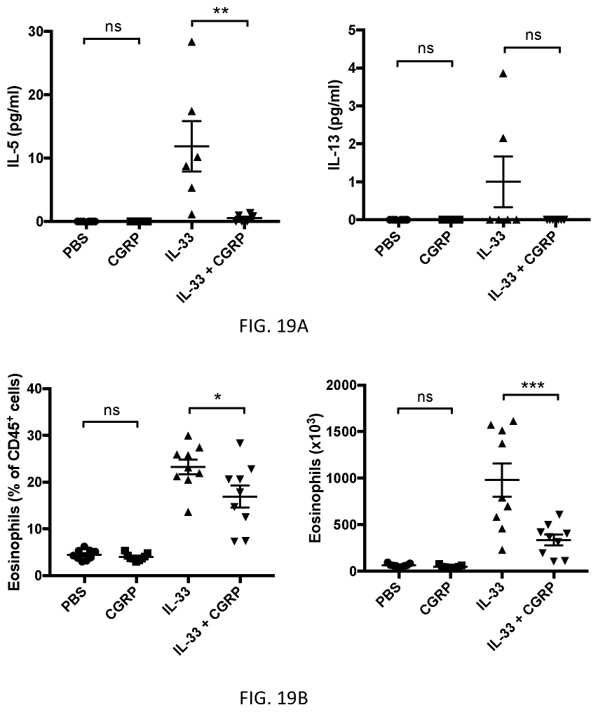

CGRP also inhibited type 2 cytokine production induced by IL-33 in vivo. Compared to IL-33 alone, co-administration of CGRP and IL-33 significantly reduced the frequency of IL-5- and IL-13-positive ILCs (FIG. 18D), 15 and Il3 transcripts were significantly reduced in lung homogenates (FIG. 18E) and IL-5 and IL-13 protein was greatly diminished in lung homogenates and bronchoalveolar lavage fluid (FIGS. 18F and 19A). Consistently, the frequencies and numbers of eosinophils in both lung tissue and bronchoalveolar lavage fluid were also significantly decreased in these conditions (FIGS. 18G and 19B). Moreover, CGRP markedly inhibited IL-33-induced perivascular and peribronchial lymphocytic infiltrates in lung sections (scored in a blinded-manner) (FIG. 18H), whereas lung sections from mice treated with CGRP alone were histologically identical to those from PBS-treated control mice (FIG. 18H). Indeed, mice that received IL-33+CGRP developed less airway hyperreactivity than mice treated with IL-33 alone (FIG. 18I). Thus, CGRP is a potent inhibitor of IL33-driven lung ILC2 responses in vivo.

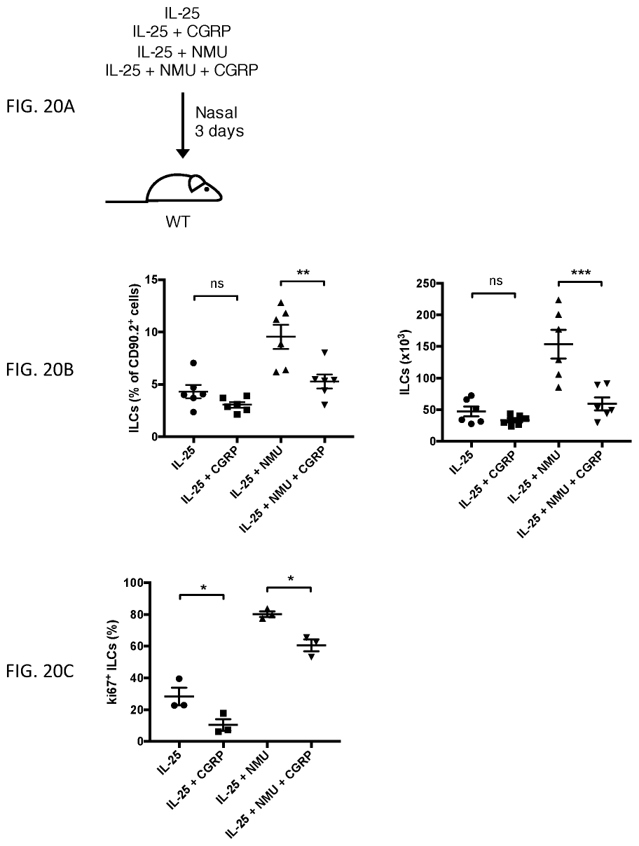

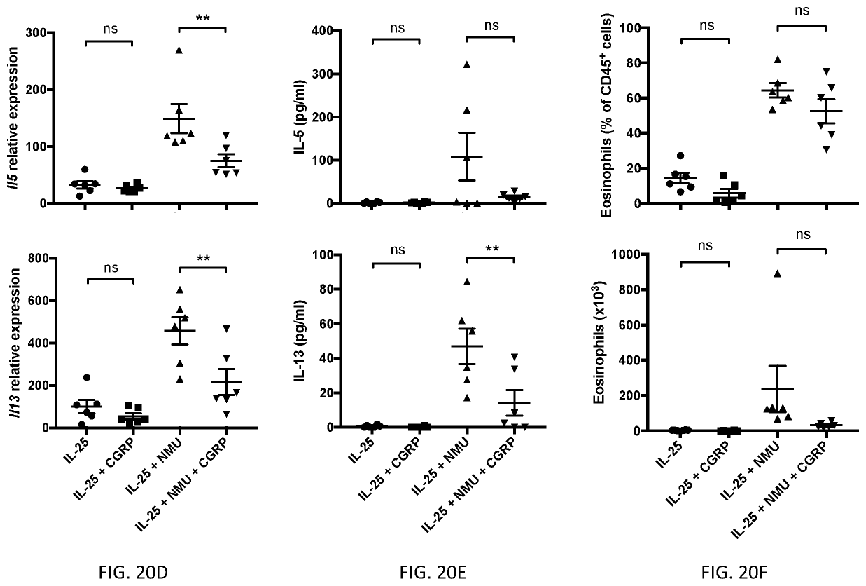

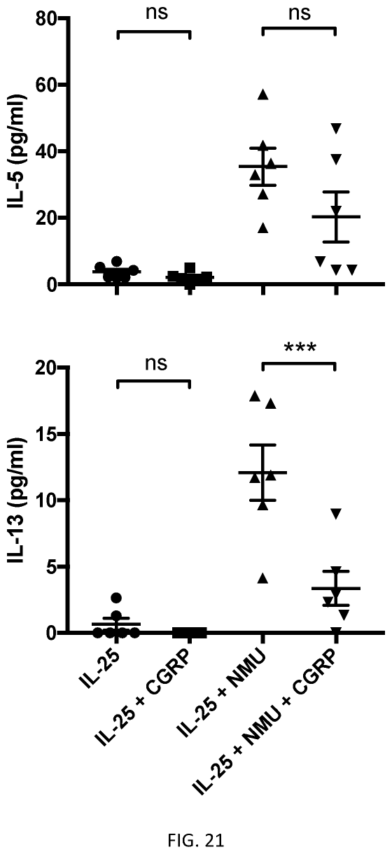

Consistent with the in vitro findings, the inhibitory effect of CGRP on lung ILC responses in vivo also extended to IL-25+NMU, albeit more mildly. To test this, Applicants treated mice nasally with IL-25, IL-25+CGRP, IL-25+NMU or IL-25+NMU+CGRP (FIG. 20A). While CGRP had no effect on ILC frequencies and numbers in IL-25-treated mice, there was a strong reduction in ILC frequencies and numbers when co-administered with IL-25+NMU (FIG. 20B). CGRP also significantly decreased ILC proliferation induced by IL-25+NMU, as assessed by intracellular staining for Ki67 (FIG. 20C). Moreover, compared to IL25+NMU, co-treatment CGRP caused diminished expression of type 2 cytokines at the mRNA level in lung tissue (FIG. 20D), reduced IL-13 protein expression in both lung tissue and bronchoalveolar lavage fluid, and showed a non-significant trend towards reduced expression of IL-5 protein (FIGS. 20F and 21) and towards decreased eosinophil frequency and numbers in the BAL (FIG. 20G). Thus, while CGRP more potently inhibit IL-33 driven lung ILC2 responses, it also inhibits IL-25+NMU-induced ILC effector function in vivo.

### CGRP Inhibition of ILC2s is Independent of T Cells

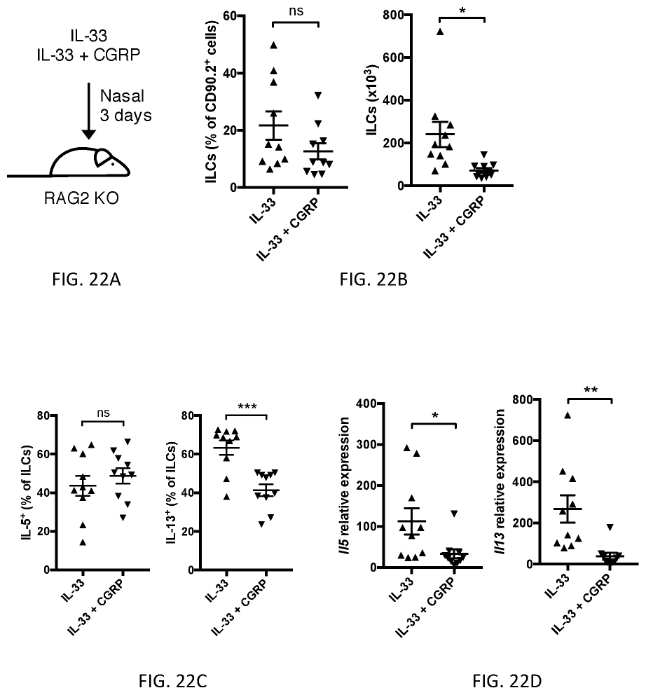

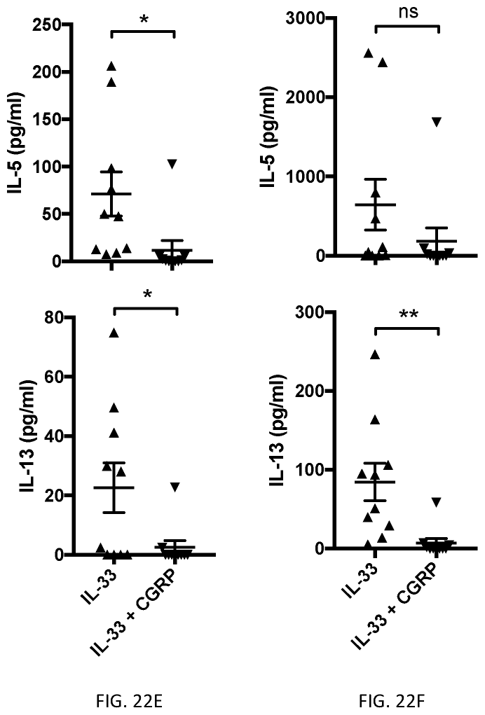

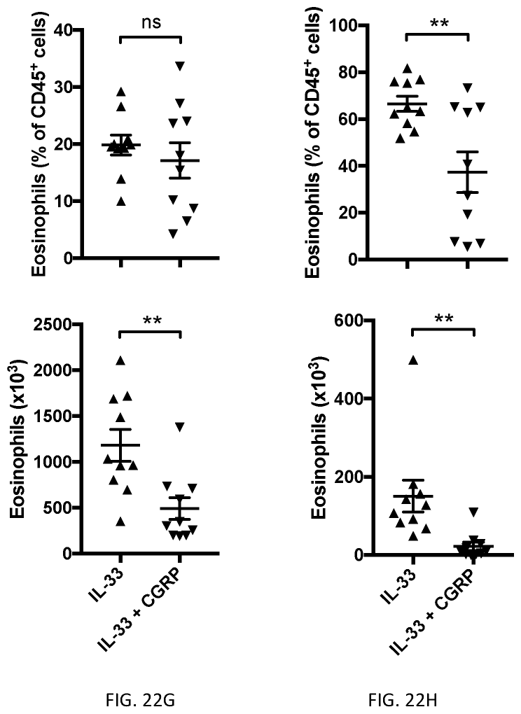

The transcriptional analysis of Ramp1 and Calcrl expression showed that the CGRP receptor is expressed in additional cell populations in the lung in addition to ILC2s (FIG. 9D), raising the possibility that the inhibitory effects of CGRP on ILC2s in vivo may be mediated indirectly. In particular, adaptive immune cells can modulate ILC2 responses, and T_{reg} cells have been shown to inhibit ILC2 activation. To test whether CGRP can inhibit alarmin driven ILC2 activation independently of adaptive immune cells, Applicants analyzed the effect of CGRP on IL-33-induced airway inflammation in RAG2 KO mice, which lack T and B cells. Mice received IL-33 or IL-33+CGRP intranasally for three consecutive days and were analyzed one day after the last treatment (FIG. 22A). There was a non-significant trend towards a reduced frequency of ILCs in mice challenged with IL-33+CGRP compared to IL-33 alone, however there was a marked reduction in the total numbers of lung ILCs in the CGRP treated mice (FIG. 22B). Although Applicants observed no difference in the frequency of IL-5-positive ILCs, the frequency of IL-13-positive ILC2s was also significantly lower in the presence of CGRP in IL-33-treated RAG2 KO mice (FIG. 22C). Compared to IL-33 alone, CGRP treatment in the presence of IL-33 also significantly reduced 115 and 1113 transcripts in lung tissue (FIG. 22D), the concentration of IL-5 and IL-13 protein in lung homogenates and BALF (FIG. 22E,F), and eosinophil numbers in both lung tissue and BALF (FIG. 20G,H). Thus, CGRP negatively regulates alarmin-driven lung-resident ILC2 responses independently of adaptive immunity by inhibiting alarmin driven ILC proliferation and altering effector cytokine production.

### Discussion

Applicants and others have recently demonstrated that neurons regulate ILC2s during allergen driven inflammatory responses and helminth infection via production of the neurotransmitters neuromedin U, vasoactive intestinal peptide, and epinephrine (Cardoso et al., 2017; Klose et al., 2017; Moriyama et al., 2018; Nussbaum et al., 2013; Talbot et al., 2015; Wallrapp et al., 2017). To elucidate if additional neuroimmune pathways or neuropeptides regulate ILC2 responses, Applicants analyzed here the expression of neuropeptide receptors on ILC2s. Applicants identified that lung-resident ILC2s highly express Ramp1, as well as Calcrl, which together encode the receptor for CGRP, and also produce CGRP itself. Strikingly, CGRP inhibits proliferation and effector function of ILC2s in vitro and induces a regulatory set of genes associated with T cell dysfunction. In vivo, treatment with CGRP reduces the severity of acute airway inflammation by inhibiting ILC2 responses, even in the absence of adaptive immune cells, indicating that CGRP is a novel negative regulator of ILC2s.

Several previous studies have investigated the role of CGRP in regulating type 2 lung inflammation using mice lacking Ramp1 or CGRP, and report decreased airways hyperreactivity following OVA sensitization and challenge (Li et al. PLOS, Aoki-Nagase et al.) (Li et al. PLOS, Aoki-Nagase et al., Mikami et al. JI 2013). Interestingly, Li et al. found that deleting Calcrl specifically in smooth muscle cells resulted in a similar decrease of airway hyperreactivity to that observed in germline Ramp1 deficient mice (Li et al. PLOS), consistent with reports that CGRP directly induced human bronchial smooth muscle contraction (Springer et al. Regulatory Peptides 2004). The effects of CGRP on smooth muscle therefore appear to be distinct from its immunomodulatory role on ILC2s. Although ILC2s expressed Ramp1 quite highly both at steady-state and during lung inflammation compared to other lung-resident immune cell types, Applicants also observed significant expression of Ramp1 and Calcrl in other cell types, further indicating that ILCs are not the only CGRP-responsive cell type in vivo.

CGRP has been previously shown to have pleiotropic effects on immune responses and to impact the function of multiple different immune and non-immune cell types. Some studies suggest a pro-inflammatory role of CGRP (Kashem et al., 2015), including through enhanced IL-5 production after short term exposure of IL-33 activated ILCs to CGRP (Sui et al., 2018). Others report an inhibitory effect, in particular, on myeloid cells (Baliu-Pique et al., 2014; Chiu et al., 2013; Harzenetter et al., 2007; Jusek et al., 2012). While Applicants also observed that CGRP enhanced IL-5 expression in ILC2s in the short term, Applicants find that even at early time points, IL-13 is inhibited, and the overall effect of CGRP both in vitro and in vivo is to inhibit ILC2-mediated inflammation.

CGRP itself is also expressed by sensory neurons and pulmonary neuroendocrine cells (PNECs), which are specialized innervated epithelial cells that can sense hypoxia and release an array of neurotransmitters and soluble mediators (Domnik and Cutz, 2011; Linnoila, 2006). A recent study highlighted that genetic ablation of PNECs resulted in decreased allergen-induced lung inflammation, and suggested it is CGRP dependent. However, deletion of PNECs could markedly alter the local lung microenvironment, such that decreased lung inflammation may be due to loss of other factors produced by these cells. The study also reports that deletion of Calcrl in Il5-expressing cells did not alter the frequency of ILC2s but reduced the frequency of lung-infiltrating CD4 T cells and eosinophils. Developing genetic approaches to target CGRP signaling in ILC2s versus Th2 cells could provide insight into how CGRP specifically affects innate versus adaptive type 2 lymphocytes.

The finding that CGRP is expressed by ILC2s in the lung suggests that CGRP may potentially act as an autocrine or paracrine regulator of ILC2 function or as a mechanism by which ILC2s modulate the responses of other CGRP-responsive cells. CGRP not only upregulated its own expression in ILC2s but also upregulated genes associated with inhibition of effector lymphocyte responses, including a negative regulatory module (Pdcd1, Tnfrsf18, Entpd1, Lilbr4, Tnfrsf9, and Icos) recently demonstrated to be regulated by IL-27 in T cells (Chihara and Madi 2018). The same co-inhibitory gene module, induced by different stimuli, may thus also operate in ILC2s and inhibit their function.

Co-treatment with IL-33 and CGRP uniquely induced expression of Foxp3, a transcription factor associated with T_{regs}. Foxp3 can directly suppress expression of effector cytokines and promote co-inhibitory receptor expression on CD4 T cells. While the role of Foxp3 expression by ILCs remains to be determined, the data raise the intriguing possibility that Foxp3 induction may also be a mechanism by which CGRP inhibits effector ILC2 responses. Moreover, others have shown that a significant fraction of tissue T_{regs} express ST2 and, in response to IL-33, express genes promoting tissue repair. Understanding how lymphocytes respond to CGRP and IL-33 may therefore help elucidate broader pathways involved in tissue tolerance and repair.

Binding of CGRP to its receptor generates cAMP (Russell et al., 2014), and chronically elevated cAMP promotes T cell anergy (Powell et al J Immunol. 1999), consistent with the observation that prolonged CGRP treatment inhibits ILC2 effector function and proliferation. In ILCs cultured with CGRP, Applicants observed increased expression of genes involved in negative feedback of cAMP signaling, including the phosphodiesterase 4D (Pde4d), which breaks down cAMP, and the transcription factor Crem (cAMP responsive element modulator), which can negatively regulates transcription of genes downstream of the cAMP pathway (Raker et al., 2016). PDE4 family inhibitors have been developed for the treatment of chronic inflammatory diseases, including atopic dermatitis, chronic obstructive pulmonary disease (COPD) and psoriatic arthritis, providing additional evidence that increases in intracellular cAMP levels can inhibit inflammation (Li et al. Front. Pharm. 2018). These data suggest that the modulation of cAMP may represent a pathway by which CGRP inhibits ILC2 activation and function.

Finally, the data show that by negatively regulating ILC2 responses, CGRP limits development of acute airway inflammation. Humanized monoclonal antibodies that inhibit either CGRP or the CGRP receptor have recently been approved as a treatment for migraine prevention (Goadsby et al., 2017). Although no significant asthma-related adverse events were reported in the phase 3 clinical trials of these antibodies, the possibility that these agents could enhance ILC2 responses and promote type 2 inflammation clearly warrants close monitoring as these agents enter widespread clinical use. This study also highlights that therapeutic activation of the CGRP receptor could be a therapeutic strategy for treating chronic allergic, inflammatory diseases, such as asthma, food allergy, and other atopic disorders.

### Methods

### Experimental Model and Subject Details

Animals. All experiments involving mice were approved by the Institutional Animal Care and Use Committee (IACUC) at Brigham and Women's Hospital. Mice were maintained in the animal facility at Brigham and Women's Hospital under specific pathogen-free conditions with food and water ad libitum and a 12-hour dark/light cycle. Mice were age- and sex-matched for experiments and were randomly assigned to experimental groups. C57BL/6J mice and RAG2 KO mice were purchased from the Jackson Laboratory. CGRP-GFP-hDTR mice were kindly provided by I. Chiu (Harvard Medical School, Boston).

Primary Cell Culture. Primary cells were cultured in a humidified incubator at 37° C. and 10% CO2 in complete medium consisting of RPMI 1640 medium (Cat #11875-119; Thermo Fisher Scientific) supplemented with 10% fetal bovine serum, 20 mM HEPES, 2 mM L-Glutamine, 1% Penicillin/Streptomycin and ß-Mercaptoethanol.

### Method Details

Isolation of lung cells for fluorescence-activated cell sorting. Mice were euthanized and perfused with 8 ml cold PBS via the right heart ventricle. Lung lobes were removed from the chest cavity and transferred into gentleMACS C tubes containing Buffer S and enzymes A and D from the lung dissociation kit (Cat #130-095-927; Miltenyi Biotec). After manual dissociation of the tissue by running program lung_01 of the automated tissue dissociator (gentleMACS; Miltenyi Biotec), and digestion at 37° C. for 25 min on a rotator, the tissue pieces were further dissociated by running program lung_02 of the automated tissue dissociator. Subsequently, the single-cell suspension was passed through a 70 um cell strainer and washed with DPBS (Cat #14190-144; Thermo Fisher Scientific) containing 0.5% bovine serum albumin (Cat #BP1600-1; Fisher Scientific) and 2 mM EDTA. After incubation of the cells with CD90.2 MicroBeads (Cat #130-049-101; Miltenyi Biotec) on ice for 17 min, cells were washed and transferred onto LS columns (Cat #130-042-401; Miltenyi Biotec) to enrich for CD90.2-positive cells by positive selection. Then, positive and negative cell fractions were stained with surface antibodies for 20 min on ice in the dark, washed and resuspended in 1-2 ml DPBS containing 0.5% bovine serum albumin and 2 mM EDTA. Cells were purified by fluorescence-activated cell sorting using a BD FACS Aria IIIu flow cytometer with 3 lasers (405 nm, 488 nm, 640 nm) or 4 lasers (405 nm, 488 nm, 561 nm and 640 nm) (BD Biosciences). For subsequent cell culture, cells were sorted into RPMI 1640 medium (Cat #11875-119; Thermo Fisher Scientific) supplemented with 10% fetal bovine serum, 20 mM HEPES, 2 mM L-Glutamine, 1% Penicillin/Streptomycin and ß-Mercaptoethanol. For RNA isolation, cells were directly sorted into RLT Plus lysis buffer (RNeasy Plus Mini Kit; Qiagen) or extraction buffer (PicoPure RNA Isolation Kit; Thermo Fisher Scientific). Debris and doublets were excluded for cell types using forward and sideward scatter. The CD90.2 positive cell fraction was used to sort innate lymphoid cells (ILCs) (7AAD−, CD45+, CD90.2+, Lineage (CD3, CD4, CD8, CD11b, CD11c, CD19, NK1.1, TCRb, TCRgd)−, CD127+ cells), ILC2s (ST2+ILCs), CD4 T cells (7AAD−, CD45+, CD3+, CD4+, TCRb+ cells) and TCRgd T cells (7AAD−, CD45+, CD3+, CD4−, TCRb−, TCRgd+). The CD90.2 negative fraction was used to sort B cells (7AAD−, CD45+, CD19+), eosinophils (7AAD−, CD45+, CD19−, CD11b+, CD11c^{low}, Siglec-F+, SSC-A^{hi}), neutrophils (7AAD−, CD45+, CD19−, CD11b+, CD11c^{low}, Siglec-F−, Ly6G+), macrophages (7AAD−, CD45+, CD11b+, CD11c+, F4/80+, Siglec-F+, MHC2+) and CD45− cells (7AAD−, CD45−). For the isolation of inflammatory ILC2s, single-cell suspension was enriched for lymphocytes by 40/70% Percoll gradient centrifugation instead of enrichment with CD90.2 beads. Inflammatory ILC2s were defined as 7AAD−, CD45+, CD127+, Lineage−, CD90.2^{int}, ST2−, KLRG1+ cells.

Culture of innate lymphoid cells. Sort-purified innate lymphoid cells (ILCs) were cultured under sterile conditions in complete medium in a humidified incubator at 37° C. and 10% CO2. ILCs were plated at a density of 3,000-5,000 ILCs per well in a 96 well round-bottom plate in cell culture medium with 20 ng/ml IL-7. Depending on the experiment, different combinations of 200 ng/ml IL-25, 200 ng/ml IL-33, 1 ug/ml NMU and 100 nM CGRP were added either at start or after overnight culture with 20 ng/ml IL-7. After 6 hours or 3 days, culture supernatant was removed and frozen at −20° C. and ILCs were lysed in Extraction Buffer (PicoPure RNA isolation Kit; Thermo Fisher Scientific), incubated at 42° C. for 30 min and frozen at −80° C.

Proliferation assay. Lung cells were isolated and enriched for CD90.2 cells as described above, followed by labeling with CellTrace Violet (Cat #C34557; Thermo Fisher Scientific) according to manufacturer's instructions and subsequently stained with antibodies. After 3-day culture of sort-purified ILCs with 20 ng/ml IL-7 either alone or in combination with 200 ng/ml IL-33, 100 pM CGRP or 100 nM CGRP followed by staining, expression of CellTrace Violet in live (7AAD−) ILCs was analyzed on a BD LSRFortessa (BD Biosciences).

Cytokine-induced airway inflammation. Mice received nasally 500 ng IL-25, 500 ng IL-33, 20 ug NMU or 6.65 ug CGRP diluted in DPBS for three consecutive days. For nasal administration, mice were lightly anesthetized with Isoflurane (Cat #07-893-1389; Patterson Veterinary). For induction of inflammatory ILC2s, mice received intraperitoneally 500 ng IL-25 for three consecutive days. One day after the last treatment, mice were euthanized and perfused with 8 ml cold PBS via the right heart ventricle. After exposure of the trachea, a small incision was made at the top of the trachea and a curved gavage needle was inserted. Lungs were washed with 1.5 ml cold PBS via the needle and the retrieved bronchoalveolar lavage fluid was centrifuged at 1300 rpm for 5 min at 4 C. After centrifugation, the supernatant was frozen at −20° C. and the cell pellet was resuspended in 250 ul complete medium and stored on ice until flow cytometric analysis. The post-caval lung lobe was transferred into 10% buffered formalin and stored at room temperature until paraffin embedding for histological analysis. The other lung lobes were dissociated using the lung dissociation kit (Cat #130-095-927; Miltenyi Biotec) and automated tissue dissociator (gentleMACS; Miltenyi Biotec) as described above with the adjustment that after running program lung_02, the single cell suspension was centrifuged at 1300 rpm for 5 min at 4° C. and 1 ml of the supernatant was frozen at −20° C. Single-cell suspension was resuspended in complete cell culture medium and stored on ice until further processing.

For RNA isolation, lung cells were centrifuged at 300 g for 6 min at 4° C., the supernatant was discarded and the cell pellet was resuspended in 600 ul RLT Buffer Plus (Qiagen RNA isolation kit), vortexed and frozen at −80° C.

For cell counts, lung cells were transferred into FACS tubes and stained with 7AAD. Precision Count Beads (Cat #424902; BioLegend) were added according to manufacturer's instructions. Cells and beads were acquired on a BD LSRFortessa (BD Biosciences) and cell numbers were calculated based on number of acquired live (7AAD−) cells and number of acquired beads.

For flow cytometric analysis, lung cells were transferred into a 96 well V-bottom plate and stained with surface antibodies for 20 min at 4° C. in the dark. Cells were washed twice with DPBS containing 2% fetal bovine serum and transferred into 1.2 ml tubes for analysis by flow cytometry. For intracellular cytokine staining, cells were incubated in complete cell culture medium with 50 ng/ml phorbol 12-myristate 13-acetate (Sigma-Aldrich), 1 uM ionomycin (Sigma-Aldrich) and GolgiStop (Cat #554724; BD Biosciences) in a humidified incubator at 37° C. and 10% CO2. After 5 hours, cells were transferred into 96 well V-bottom plate, stained with Fixable Viability Dye eFluor 506 (Cat #65-0866-14; Thermo Fisher) and surface antibodies for 20 min at 4° C. in the dark and washed twice with DPBS containing 2% fetal bovine serum. Then, cells were incubated with BD Cytofix/Cytoperm Buffer (Cat #51-2090KZ; BD Biosciences) for 20 min, followed by a wash with BD Perm/Wash Buffer (Cat #51-2091KZ; BD Biosciences). Subsequently, cells were incubated with antibodies targeting intracellular proteins diluted in BD Perm/Wash Buffer for 20 min and after two wash steps, transferred into 1.2 ml tubes for analysis by flow cytometry. For ki67 staining, cells were stained for surface antibodies as described above, fixed with a solution of Fixation/Permeabilization Concentrate (Cat #00-5123-43; Invitrogen) and Fixation/Perm Diluent (Cat #00-5223-56; Invitrogen) for 20 min, washed with Permeabilization Buffer (Cat #00-8333-56; Invitrogen). Then, cells were incubated with ki67 antibody diluted in Permeabilization Buffer for 20 min, washed and transferred into FACS tubes. Cells were analyzed on a BD LSRFortessa (BD Biosciences) flow cytometer with 5 lasers (355 nm, 405 nm, 488 nm, 561 nm and 640 nm). Data was analyzed using FlowJo v10.5.0 software and cell populations were gated as described previously (Wallrapp 2017).

RNA isolation and cDNA synthesis. RNA was isolated from lung cells and immune cell populations sorted from naïve and IL-33-treated mice using the Qiagen RNeasy Plus Mini Kit (Cat #74134; Qiagen) according to manufacturer's instructions. RNA concentration and purity were determined with a NanoDrop spectrophotometer (Thermo Fisher Scientific) and equal amounts of RNA were reverse transcribed to cDNA using the iScript cDNA Synthesis Kit (Cat #1708891; Bio-Rad). RNA was isolated from cultured ILCs or ex vivo sort-purified ILCs with the PicoPure RNA isolation kit (Cat #KIT0204; Thermo Fisher Scientific) according to manufacturer's instructions and subsequently reverse transcribed to cDNA with the SuperScript IV VILO Master Mix (Cat #11756050; Thermo Fisher Scientific). To analyze gene expression, cDNA, TaqMan Gene Expression Assay for the respective gene and a housekeeping gene were added to the TaqMan Fast Advanced Master Mix (Cat #4444557; Thermo Fisher Scientific) and quantitative real-time PCR was performed with a ViiA 7 system (Thermo Fisher Scientific). Gene expression was normalized to expression of the housekeeping gene Actin-b. The following TaqMan probes were used: Il5 (Mm00439646_m1), Il3 (Mm00434204_m1), Ramp1 (Mm00489796_m1), Ramp2 (Mm00490256_g1), Ramp3 (Mm00840142_m1), Calcrl (Mm00516986_m1), Calca (Mm01274759_g1), Areg (Mm00437583_m1) and Actb (Cat #4352341E; Thermo Fisher Scientific).

Bead-based immunoassay. Cytokine concentrations in bronchoalveolar lavage fluid, lung tissue and ILC culture supernatant were determined using the LEGENDplex mouse Th cytokine panel (Cat #740005; BioLegend) or mouse Th2 cytokine panel (Cat #740027; BioLegend) according to manufacturer's instructions. Samples were acquired using a BD LSRFortessa flow cytometer (BD Biosciences) and analyzed with the LEGENDplex Software v7.1.

Methacholine assay. Mice were anesthetized with Pentobarbital and a 20G needle was inserted into the trachea and subsequently connected to a flexiVent FX1 instrument (SCIREQ). Mice were exposed to increasing doses of aerosolized methacholine (0, 3, 10, 30, 100 mg/ml diluted in DPBS) and airway resistance was measured. For each dose the airway resistance was calculated as the mean of 8 measurements.

Histology. Lung tissue was fixed in 10% buffered formalin at room temperature and embedded in paraffin. After sectioning, lung slices were stained with hematoxylin and eosin (H&E) and scored for severity of airway inflammation by a histopathologist in a blinded manner according to the following scoring system: 0, normal; 1, very mild; 2, mild; 3, moderate; 4, severe.

Quantification and Statistical Analysis. Statistical analysis was performed with GraphPad Prism software version 7.0a (GraphPad). Data are shown as mean+/−SEM. Statistical significance was determined using unpaired two-tailed t test (when comparing two groups) or a one-way ANOVA with Tukey's multiple comparisons test (for the comparison of three or more groups) unless otherwise indicated.

### REFERENCES

Baliu-Pique, M., Jusek, G., and Holzmann, B. (2014). Neuroimmunological communication via CGRP promotes the development of a regulatory phenotype in TLR4-stimulated macrophages. European journal of immunology 44, 3708-3716.
Branchfield, K., Nantie, L., Verheyden, J. M., Sui, P., Wienhold, M. D., and Sun, X. (2016). Pulmonary neuroendocrine cells function as airway sensors to control lung immune response. Science 351, 707-710.
Cardoso, V., Chesne, J., Ribeiro, H., Garcia-Cassani, B., Carvalho, T., Bouchery, T., Shah, K., Barbosa-Morais, N. L., Harris, N., and Veiga-Fernandes, H. (2017). Neuronal regulation of type 2 innate lymphoid cells via neuromedin U. Nature 549, 277-281.
Chiu, I. M., Heesters, B. A., Ghasemlou, N., Von Hehn, C. A., Zhao, F., Tran, J., Wainger, B., Strominger, A., Muralidharan, S., Horswill, A. R., et al. (2013). Bacteria activate sensory neurons that modulate pain and inflammation. Nature 501, 52-57.
Domnik, N. J., and Cutz, E. (2011). Pulmonary neuroepithelial bodies as airway sensors: putative role in the generation of dyspnea. Curr Opin Pharmacol 11, 211-217.
Goadsby, P. J., Reuter, U., Hallström, Y., Broessner, G., Bonner, J. H., Zhang, F., Sapra, S., Picard, H., Mikol, D. D., and Lenz, R. A. (2017). A Controlled Trial of Erenumab for Episodic Migraine. New England Journal of Medicine 377, 2123-2132.
Harzenetter, M. D., Novotny, A. R., Gais, P., Molina, C. A., Altmayr, F., and Holzmann, B. (2007). Negative Regulation of TLR Responses by the Neuropeptide CGRP Is Mediated by the Transcriptional Repressor ICER. The Journal of Immunology 179, 607-615.
Huang, Y., Guo, L., Qiu, J., Chen, X., Hu-Li, J., Siebenlist, U., Williamson, P. R., Urban, J. F., Jr., and Paul, W. E. (2015). IL-25-responsive, lineage-negative KLRG1(hi) cells are multipotential ‘inflammatory’ type 2 innate lymphoid cells. Nature immunology 16, 161-169.
Huang, Y., Mao, K., Chen, X., Sun, M. A., Kawabe, T., Li, W., Usher, N., Zhu, J., Urban, J. F., Jr., Paul, W. E., et al. (2018). SiP-dependent interorgan trafficking of group 2 innate lymphoid cells supports host defense. Science 359, 114-119.
Jusek, G., Reim, D., Tsujikawa, K., and Holzmann, B. (2012). Deficiency of the CGRP receptor component RAMP1 attenuates immunosuppression during the early phase of septic peritonitis. Immunobiology 217, 761-767.
Kashem, S. W., Riedl, M. S., Yao, C., Honda, C. N., Vulchanova, L., and Kaplan, D. H. (2015). Nociceptive Sensory Fibers Drive Interleukin-23 Production from CD301b+ Dermal Dendritic Cells and Drive Protective Cutaneous Immunity. Immunity 43, 515-526.
Klose, C. S. N., Mahlakoiv, T., Moeller, J. B., Rankin, L. C., Flamar, A. L., Kabata, H., Monticelli, L. A., Moriyama, S., Putzel, G. G., Rakhilin, N., et al. (2017). The neuropeptide neuromedin U stimulates innate lymphoid cells and type 2 inflammation. Nature 549, 282-286.
Lambrecht, B. N., and Hammad, H. (2015). The immunology of asthma. Nature immunology 16, 45-56.
Linnoila, R. I. (2006). Functional facets of the pulmonary neuroendocrine system. Laboratory Investigation 86, 425-444.
Moriyama, S., Brestoff, J. R., Flamar, A. L., Moeller, J. B., Klose, C. S. N., Rankin, L. C., Yudanin, N. A., Monticelli, L. A., Putzel, G. G., Rodewald, H. R., et al. (2018). beta2-adrenergic receptor-mediated negative regulation of group 2 innate lymphoid cell responses. Science 359, 1056-1061.
Moro, K., Yamada, T., Tanabe, M., Takeuchi, T., Ikawa, T., Kawamoto, H., Furusawa, J., Ohtani, M., Fujii, H., and Koyasu, S. (2010). Innate production of T(H)2 cytokines by adipose tissue-associated c-Kit(+)Sca-1(+) lymphoid cells. Nature 463, 540-544.
Neill, D. R., Wong, S. H., Bellosi, A., Flynn, R. J., Daly, M., Langford, T. K., Bucks, C., Kane, C. M., Fallon, P. G., Pannell, R., et al. (2010). Nuocytes represent a new innate effector leukocyte that mediates type-2 immunity. Nature 464, 1367-1370.
Nussbaum, J. C., Van Dyken, S. J., von Moltke, J., Cheng, L. E., Mohapatra, A., Molofsky, A. B., Thornton, E. E., Krummel, M. F., Chawla, A., Liang, H. E., et al. (2013). Type 2 innate lymphoid cells control eosinophil homeostasis. Nature 502, 245-248.
Raker, V. K., Becker, C., and Steinbrink, K. (2016). The cAMP Pathway as Therapeutic Target in Autoimmune and Inflammatory Diseases. Frontiers in immunology 7, 123.
Russell, F. A., King, R., Smillie, S. J., Kodji, X., and Brain, S. D. (2014). Calcitonin gene-related peptide: physiology and pathophysiology. Physiol Rev 94, 1099-1142.
Sui, P., Wiesner, D. L., Xu, J., Zhang, Y., Lee, J., Van Dyken, S., Lashua, A., Yu, C., Klein, B. S., Locksley, R. M., et al. (2018). Pulmonary neuroendocrine cells amplify allergic asthma responses. Science 360.
Talbot, S., Abdulnour, R E., Burkett, P. R., Lee, S., Cronin, S. J., Pascal, M. A., Laedermann, C., Foster, S. L., Tran, J. V., Lai, N., et al. (2015). Silencing Nociceptor Neurons Reduces Allergic Airway Inflammation. Neuron 87, 341-354.
Wallrapp, A., Riesenfeld, S. J., Burkett, P. R., Abdulnour, R. E., Nyman, J., Dionne, D., Hofree, M., Cuoco, M. S., Rodman, C., Farouq, D., et al. (2017). The neuropeptide NMU amplifies ILC2-driven allergic lung inflammation. Nature 549, 351-356.
Wallrapp, A., Riesenfeld, S. J., Burkett, P. R., and Kuchroo, V. K. (2018). Type 2 innate lymphoid cells in the induction and resolution of tissue inflammation. Immunological reviews 286, 53-73.
Yu, S., Kim, H. Y., Chang, Y. J., DeKruyff, R. H., and Umetsu, D. T. (2014). Innate lymphoid cells and asthma. The Journal of allergy and clinical immunology 133, 943-950; quiz 951.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating a disease associated with an innate lymphoid cell (ILC) Type 2 inflammatory response comprising administering to a subject in need thereof a therapeutically effective amount of α-CGRP or functional derivative thereof; or a α-CGRP receptor agonist.

2. The method of claim 1, wherein the innate lymphoid cell (ILC) Type 2 inflammatory response is an IL-33 mediated response.

3. The method of claim 1, wherein the innate lymphoid cell (ILC) Type 2 inflammatory response is an IL-25+ neuromedin U (NMU) mediated response.

4. The method of claim 1, wherein the innate lymphoid cell (ILC) Type 2 inflammatory response comprises the release of a neurotransmitter from stimulated neurons.

5. The method of claim 4, wherein the neurotransmitter is NMU or vasoactive intestinal peptide (VIP).

6. The method of claim 1, further comprising administering a glucocorticoid, wherein the glucocorticoid is co-administered or administered after the therapeutically effective amount of α-CGRP or derivative thereof, or the α-CGRP receptor agonist.

7. The method of claim 1, further comprising administering one or more agonists of one or more genes selected from the group consisting of PD-1, TIM-3, LILRB4, CD39, GITR, wherein the one or more agonists are co-administered or administered after the therapeutically effective amount of α-CGRP or derivative thereof, or the α-CGRP receptor agonist.

8. The method of any of claims 1 to 7, wherein the agonist is an agonist antibody, small molecule or ligand, such as a GITR agonist antibody, GITRL, or PD-L1.

9. The method of any of claims 1 to 8, wherein the disease is an allergic inflammatory disease.

10. The method of claim 9, wherein the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

11. The method of claim 10, wherein the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma.

12. The method of claim 10, wherein the allergy is to an allergen selected from the group consisting of food, pollen, mold, dust mites, animals, and animal dander.

13. The method of claim 10, wherein IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

14. The method of claim 10, wherein the arthritis is selected from the group consisting of osteoarthritis, rheumatoid arthritis and psoriatic arthritis.

15. The method of any of claims 1 to 14, wherein the treatment is administered to a mucosal surface.

16. The method of claim 15, wherein the treatment is administered to the lung, nasal passage (e.g., intranasally), trachea, gut, intestine, or esophagus.

17. The method of any of claims 1 to 16, wherein the treatment is administered by aerosol inhalation.

18. The method of any of claims 1 to 16, wherein the treatment is administered by a time release composition.

19. A method of treating a disease by enhancing an innate lymphoid cell (ILC) Type 2 inflammatory response comprising administering to a subject in need thereof a therapeutically effective amount of an agent capable of antagonizing α-CGRP receptor signaling or blocking the α-CGRP receptor interaction with α-CGRP.

20. The method of claim 19, wherein the agent comprises a therapeutic antibody, antibody fragment, antibody-like protein scaffold, aptamer, nucleic acid molecule, genetic modifying agent, protein or small molecule.

21. The method of claim 20, wherein the agent binds to the α-CGRP receptor or α-CGRP.

22. The method of any of claims 19 to 21, further comprising administering one or more inhibitors of one or more genes selected from the group consisting of PD-1, TIM-3, LILRB4, CD39, GITR and PD-L1.

23. The method of claim 22, wherein the one or more inhibitors comprises an antibody or small molecule specific for PD-1, TIM-3, LILRB4, CD39, GITR, or PD-Li.

24. The method of claim 22, wherein the one or more inhibitors comprises Nivolumab, Pembrolizumab, Atezolizumab, 6-N,N-Diethyl-d-β-γ-dibromomethylene adenosine triphosphate (ARL 67156), 8-thiobutyladenosine 5′-triphosphate (8-Bu-S-ATP), polyoxymetate-1 (POM-1), or α,β-methylene ADP (APCP).

25. The method of any of claims 19 to 24, wherein the disease is cancer or an infection.

26. A method of treatment for a subject in need thereof suffering from allergic inflammation comprising:
a. detecting in ILC2s obtained from the subject the expression or activity of an innate lymphoid cell type 2 inflammatory gene signature comprising one or more genes or polypeptides selected from the group consisting of:
i. Sos1, Egfr, Tph1, P2ry1, Far1, Plin2, Alox5, Pparg, Ikzf1, Ier3, Rilpl2, Stap1, Gimap5, Odc1, Smox, Calca, Ramp3, Rora, Il7r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd4l, Jag1, Zfp36l1, Lmo4, II13, I16, Il4ra, Prdm1, Arg1, Zeb2, Srgap3, Ptger4, Pcsk1, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1 and Serpinb9; or
ii. Fosb, Btg2, Lpcat2, Sdc4, Csf2, Dgat2, Calca, Areg, Pim2, Zfp36l1, Nr4a1, Cd81, Ly6a, Lgmn, Il13, Il5, Klrg1, Batf, Pycard, Pdcd1, Lgals3, Anaxa2, Ctla4, Il1r2, Tox2, Tnfrsf8, Mt1, Tff1, Lilrb4a and H2-Ab1; or
iii. Calca, Areg, Anxa1, Anxa2, Ccl1, Ccl5, Ccr2, Ccr7, Ccr8, Cd200r1, Cd3d, Cd47, Cd48, Cd81, Csf2, Ctla4, Fas, H2-Aa, H2-Ab1, H2-Q8, H2-T23, Il13, Il1r2, Il2rb, Il5, Il6, Klrg1, Lat, Lgals3, Lilrb4a, Ltb, Mif, Ms4a4b, Nmur1, Pdcd1, Pgk1, Ptger2, Ramp1, Sdc4, Sema4a, Sepp1, Stab2, Tff1, Tmem176a, Tnfrsf4, Tnfrsf8, Tnfsf8, Vsir, Nmu, 2810417H13Rik, AA467197, Alox5, Arg1, Atf4, Batf, Bcl2a1b, Blk, Btg1, Cox5b, Cox6c, Crip1, Dgat1, Dgat2, Dusp1, Ets1, Fos, Fosb, Furin, Gadd45b, Gsto1, Hint1, Ier2, Irf4, Klf3, Klf4, Lgmn, Lpcat2, Mcm3, Mt1, My16, Ndufa4, Nfkbia, Nfkbid, Nfkbiz, Nop56, Nr4a1, Prdx4, S100a4, S100a6, Serpinb6a, Snrpd3, Sptssa, Tph1, Vim, Zfp36 and Zfp36l1; or
iv. Anxa2, Lgals3, Ctla4, Batf, Cd47, Tnfrsf8, AA467197, S100a6, Prdx4, Gsto1, Illr2, Lgmn, Mt1, Tff1, Ccr7, Irf4, 116, Tnfrsf4, H2-T23, Lilrb4a, Fas, Ets1, Ramp1, Nmur1, Dgat2, Calca, Ccl5, Btg1, Nr4a1, Klf3, Klf4, Csf2, Stab2, Sdc4, Ccr2, Fosb, Zfp36l1, Lpcat2 and Ltb; and
b. treating the subject with α-CGRP or functional derivative thereof, or an agonist of the α-CGRP receptor if the inflammatory signature is detected.

27. A method of detecting and/or monitoring an immune response comprising detecting in ILC2s the expression of one or more genes selected from the group consisting of:
a. Calca, Ramp1, Calcrl, and Ramp3; or
b. Sos1, Egfr, Tph1, P2ry1, Far1, Plin2, Alox5, Pparg, Ikzf1, Ier3, Rilpl2, Stap1, Gimap5, Odc1, Smox, Calca, Ramp3, Rora, I17r, Ier2, Ltb, Ccl1, Ccr7, Sel1, S1pr1, Crem, Fosl2, Epas1, Hif1a, Egln3, Hilpda, Dgat1, Dgat2, Lpcat2, Fa2h, Tnf, Il17f, Ifngr1, Il17rb, Crlf2, Areg, Cd69, Nr4a1, Kit, Irf5, Rgs6, Rasgrp1, Plcg1, Pde4d, Nedd4l, Jag1, Zfp3611, Lmo4, Il13, Il6, Il4ra, Prdm1, Arg1, Zeb2, Srgap3, Ptger4, Pcsk1, Foxp3, Nfil3, Entpd1, Tnfrsf18, Tnfrsf9, Tnfaip3, Icos, Havcr2, Fgl2, Pdcd1, Nr3c1, Ccl22, Ikzf3, Ccr4, Gp49a, Lilrb4, Gadd45b, Serpine1 and Serpinb9; or
c. Arg1, Ly6a, Stab1, Ptger4, Maf, Tph1, Traip, Kdm8, Birc5, Mki67, Crem, Fosl2, Odc1, Smox, Nr3c1, Rora, Lmo4, Ikzf3, Il7r, Il1rl1, Crlf2, Il17rb, Xbp1, Itk, Ccr4, Icos, Irf4, Pdcd1, Ctla2a, Fgl2, Gp49a, Nt5e, Tnfrsf9, Tnfrsf18, Lilrb4, Tnfaip3, Pde4d, Nmb, Calca, Ramp3, Serpinb9, Hif1a, Egln3.

28. The method of claim 27, wherein the immune response is monitored in a subject administered an allergic challenge.

29. The method claim 27, wherein the immune response is monitored in a subject undergoing treatment for an allergic inflammatory disease.

30. The method of claim 29, wherein the allergic inflammatory disease is selected from the group consisting of asthma, allergy, allergic rhinitis, allergic airway inflammation, atopic dermatitis (AD), chronic obstructive pulmonary disease (COPD), inflammatory bowel disease (IBD), multiple sclerosis, arthritis, psoriasis, eosinophilic esophagitis, eosinophilic pneumonia, eosinophilic psoriasis, hypereosinophilic syndrome, graft-versus-host disease, uveitis, cardiovascular disease, pain, multiple sclerosis, lupus, vasculitis, chronic idiopathic urticaria and Eosinophilic Granulomatosis with Polyangiitis (Churg-Strauss Syndrome).

31. The method of claim 30, wherein the asthma is selected from the group consisting of allergic asthma, non-allergic asthma, severe refractory asthma, asthma exacerbations, viral-induced asthma or viral-induced asthma exacerbations, steroid resistant asthma, steroid sensitive asthma, eosinophilic asthma and non-eosinophilic asthma.

32. The method of claim 30, wherein the allergy is to an allergen selected from the group consisting of foods, pollen, mold, dust mites, animals, and animal dander.

33. The method of claim 30, wherein IBD comprises a disease selected from the group consisting of ulcerative colitis (UC), Crohn's Disease, collagenous colitis, lymphocytic colitis, ischemic colitis, diversion colitis, Behcet's syndrome, infective colitis, indeterminate colitis, and other disorders characterized by inflammation of the mucosal layer of the large intestine or colon.

34. The method of claim 30, wherein the arthritis is selected from the group consisting of osteoarthritis, rheumatoid arthritis and psoriatic arthritis.

35. The method claim 27, wherein the immune response is monitored in a subject suffering from cancer.

36. A medical device comprising a therapeutically effective amount of α-CGRP or functional derivative thereof.

37. The device of claim 36, further comprising a glucocorticoid.

38. The device of claim 36 or 37, wherein the device is a nasal spray.
